SEQUENCE LISTING (33 sequences)

SEQ ID NO: 1 - RNA, 510 nt, Homo sapiens
  source 1..510: mol_type = mRNA
atgaagacccagagggacggccacagcctgggcaggtggagcctggtgctgctgctgctgggcctggtgatgcccctggccatcatcgcccaggtgctgagctacaaggaggccgtgctgagggccatcgacggcatcaaccagaggagcagcgacgccaacctgtacaggctgctggacctggaccccaggcccaccatggacggcgaccccgacacccccaagcccgtgagcttcaccgtgaaggagaccgtgtgccccaggaccacccagcagagccccgaggactgcgacttcaagaaggacggcctggtgaagaggtgcatgggcaccgtgaccctgaaccaggccaggggcagcttcgacatcagctgcgacaaggacaacaagaggttcgccctgctgggcgacttcttcaggaagagcaaggagaagatcggcaaggagttcaagaggatcgtgcagaggatcaaggacttcctgaggaacctggtgcccaggaccgagagc

SEQ ID NO: 2 - DNA, 510 bp, Homo sapiens
  source 1..510: mol_type = genomic DNA
atgaagacccagagggacggccacagcctgggcaggtggagcctggtgctgctgctgctgggcctggtgatgcccctggccatcatcgcccaggtgctgagctacaaggaggccgtgctgagggccatcgacggcatcaaccagaggagcagcgacgccaacctgtacaggctgctggacctggaccccaggcccaccatggacggcgaccccgacacccccaagcccgtgagcttcaccgtgaaggagaccgtgtgccccaggaccacccagcagagccccgaggactgcgacttcaagaaggacggcctggtgaagaggtgcatgggcaccgtgaccctgaaccaggccaggggcagcttcgacatcagctgcgacaaggacaacaagaggttcgccctgctgggcgacttcttcaggaagagcaaggagaagatcggcaaggagttcaagaggatcgtgcagaggatcaaggacttcctgaggaacctggtgcccaggaccgagagc

SEQ ID NO: 3 - protein, 170 aa, Homo sapiens
MKTQRDGHSLGRWSLVLLLLGLVMPLAIIAQVLSYKEAVLRAIDGINQRSSDANLYRLLDLDPRPTMDGDPDTPKPVSFTVKETVCPRTTQQSPEDCDFKKDGLVKRCMGTVTLNQARGSFDISCDKDNKRFALLGDFFRKSKEKIGKEFKRIVQRIKDFLRNLVPRTES

SEQ ID NO: 4 - RNA, 111 nt, Homo sapiens
  source 1..111: mol_type = mRNA
ctgctgggcgacttcttcaggaagagcaaggagaagatcggcaaggagttcaagaggatcgtgcagaggatcaaggacttcctgaggaacctggtgcccaggaccgagagc

SEQ ID NO: 5 - DNA, 111 bp, Homo sapiens
  source 1..111: mol_type = genomic DNA
ctgctgggcgacttcttcaggaagagcaaggagaagatcggcaaggagttcaagaggatcgtgcagaggatcaaggacttcctgaggaacctggtgcccaggaccgagagc

SEQ ID NO: 6 - protein, 37 aa, Homo sapiens
LLGDFFRKSKEKIGKEFKRIVQRIKDFLRNLVPRTES

SEQ ID NO: 7 - RNA, 99 nt, Mus musculus
  source 1..99: mol_type = mRNA
ggcctgctgaggaagggcggcgagaagatcggcgagaagctgaagaagatcggccagaagatcaagaacttcttccagaagctggtgccccagcccgag

SEQ ID NO: 8 - DNA, 99 bp, Mus musculus
  source 1..99: mol_type = genomic DNA
ggcctgctgaggaagggcggcgagaagatcggcgagaagctgaagaagatcggccagaagatcaagaacttcttccagaagctggtgccccagcccgag

SEQ ID NO: 9 - protein, 33 aa, Mus musculus
GLLRKGGEKIGEKLKKIGQKIKNFFQKLVPQPE

SEQ ID NO: 10 - RNA, 114 nt, Mus musculus
  source 1..114: mol_type = mRNA
atcagcaggctggccggcctgctgaggaagggcggcgagaagatcggcgagaagctgaagaagatcggccagaagatcaagaacttcttccagaagctggtgccccagcccgag

SEQ ID NO: 11 - DNA, 114 bp, Mus musculus
  source 1..114: mol_type = genomic DNA
atcagcaggctggccggcctgctgaggaagggcggcgagaagatcggcgagaagctgaagaagatcggccagaagatcaagaacttcttccagaagctggtgccccagcccgag

SEQ ID NO: 12 - protein, 38 aa, Mus musculus
ISRLAGLLRKGGEKIGEKLKKIGQKIKNFFQKLVPQPE

SEQ ID NO: 13 - protein, 38 aa, Homo sapiens
MGLCPGYKIPQKILDSLDNSFKLFANLLFRFPEEITQQ

SEQ ID NO: 14 - DNA, 49 bp, synthetic construct
  source 1..49: mol_type = other DNA
ctctctacggctaacctgaatggactacgacatagtctagtccgccaag

SEQ ID NO: 15 - DNA, 61 bp, synthetic construct
  source 1..61: mol_type = other DNA
gggcccctataactctctacggctaacctgaatggactacgacatagtctagtccgccaag

SEQ ID NO: 16 - DNA, 80 bp, synthetic construct
  source 1..80: mol_type = other DNA
tcattcagctacctgagaggggcccctataactctctacggctaacctgaatggactacgacatagtctagtccgccaag

SEQ ID NO: 17 - DNA, 575 bp, synthetic construct
  source 1..575: mol_type = other DNA
cccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaacc

SEQ ID NO: 18 - DNA, 101 bp, synthetic construct
  source 1..101: mol_type = other DNA
actctagctagcagtgttaaatcattcagctacctgagaggggcccctataactctctacggctaacctgaatggactacgacatagtctagtccgccaag

SEQ ID NO: 19-20 - skipped (2 entries)

SEQ ID NO: 21 - DNA, 10 bp, synthetic construct
  source 1..10: mol_type = other DNA
gcatatgact

SEQ ID NO: 22 - DNA, 94 bp, synthetic construct
  source 1..94: mol_type = other DNA
cccctccccgacacggattggctggtgtagtgggcgcggccagagaccacccagcgcccgaccccccctccccacaaacacggggggcgtccct

SEQ ID NO: 23 - DNA, 10 bp, synthetic construct
  source 1..10: mol_type = other DNA
ccccggcgcc

SEQ ID NO: 24 - skipped

SEQ ID NO: 25 - DNA, 26 bp, synthetic construct
  source 1..26: mol_type = other DNA
ccaaaggctcttttcagagccaccca

SEQ ID NO: 26 - DNA, 44 bp, synthetic construct
  source 1..44: mol_type = other DNA
ataggcggcgcatgagagaagcccagaccaattacctacccaaa

SEQ ID NO: 27 - DNA, 319 bp, synthetic construct
  source 1..319: mol_type = other DNA
ccgctacgccccaatgatccgaccagcaaaactcgatgtacttccgaggaactgatgtgcataatgcatcaggctggtacattagatccccgcttaccgcgggcaatatagcaacactaaaaactcgatgtacttccgaggaagcgcagtgcataatgctgcgcagtgttgccacataaccactatattaaccatttatctagcggacgccaaaaactcaatgtatttctgaggaagcgtggtgcataatgccacgcagcgtctgcataacttttattatttcttttattaatcaacaaaattttgtttttaacatttc

SEQ ID NO: 28 - DNA, 70 bp, synthetic construct
  source 1..70: mol_type = other DNA
gggcccctataactctctacggctaacctgaatggacctgaatggactacgacatagtctagtccgccaa

SEQ ID NO: 29 - DNA, 36 bp, synthetic construct
  source 1..36: mol_type = other DNA
aaaaaaaaaaaaaaaaaaaaaaaaaaaaaaaaaaaa

SEQ ID NO: 30 - DNA, 9631 bp, synthetic construct
  source 1..9631: mol_type = other DNA
gataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatggagaaaatagtgcttcttcttgcaatagtcagccttgttaaaagtgatcagatttgcattggttaccatgcaaacaactcgacagagcaggttgacacaataatggaaaagaacgtcactgttacacacgcccaagacatactggaaaagacacacaacgggaaactctgcgatctagatggagtgaagcctctaattttaagagattgtagtgtagctggatggctcctcgggaacccaatgtgtgacgaattcctcaatgtgccggaatggtcttacatagtggagaagatcaatccagccaatgacctctgttacccagggaatttcaacgactatgaagaactgaaacacctattgagcagaataaaccattttgagaaaattcagatcatccccaaaagttcttggtcagatcatgaagcctcagcaggggtgagctcagcatgtccataccagggaaggtcctccttttttagaaatgtggtatggcttatcaaaaaggacaatgcatacccaacaataaagagaagttacaataataccaaccaagaagatcttttggtattgtgggggattcaccatccaaatgatgcggcagagcagacaaggctctatcaaaacccaactacctatatttccgttgggacatcaacactaaaccagagattggtaccaaaaatagccactagatctaaggtaaacgggcaaagtggaaggatggagttcttttggacaattttaaaaccgaatgatgcaataaactttgagagtaatggaaatttcattgctccagaaaatgcatacaaaattgtcaagaaaggggactcaacaattatgaaaagtgagttggaatatggtaactgcaacaccaagtgtcaaactccaataggggcgataaactctagtatgccattccacaacatccaccctctcaccatcggggaatgccccaaatatgtgaaatcaagcagattagtccttgctactgggctcagaaatagccctcaacgagagacacgaggactatttggagctatagcaggttttatagagggaggatggggaatggtagatggttggtatgggtaccaccatagcaacgagcaggggagtgggtacgctgcagacaaagaatccactcaaaaggcaatagatggagtcaccaataaggtcaactcgatcattgacaaaatgaacactcagtttgaggctgttggaagggaatttaataacttagaaaggagaatagaaaatttaaacaagaagatggaagacggattcctagatgtctggacttataatgctgaacttctggttctcatggaaaatgagagaactctagactttcatgactcaaatgtcaagaacctttacgacaaggtccgactacagcttagggataatgcaaaggagcttggtaacggttgtttcgagttctatcacagatgtgataatgaatgtatggaaagtgtaagaaacggaacgtatgactacccgcagtattcagaagaagcaagattaaaaagagaggaaataagtggagtaaaattggaatcaataggaacttaccaaatactgtcaatttattcaacagtggcgagctccctagcactggcaatcatggtggctggtctatctttatggatgtgctccaatggatcgttacaatgcagaatttgcatttaaggcgcgcccacccagcggccgcccgctacgccccaatgatccgaccagcaaaactcgatgtacttccgaggaactgatgtgcataatgcatcaggctggtacattagatccccgcttaccgcgggcaatatagcaacactaaaaactcgatgtacttccgaggaagcgcagtgcataatgctgcgcagtgttgccacataaccactatattaaccatttatctagcggacgccaaaaactcaatgtatttctgaggaagcgtggtgcataatgccacgcagcgtctgcataacttttattatttcttttattaatcaacaaaattttgtttttaacatttcaaaaaaaaaaaaaaaaaaaaaaaaaaaaaaaaaaaa

SEQ ID NO: 31 - RNA, 9631 nt, synthetic construct
  source 1..9631: mol_type = other RNA
gataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatggagaaaatagtgcttcttcttgcaatagtcagccttgttaaaagtgatcagatttgcattggttaccatgcaaacaactcgacagagcaggttgacacaataatggaaaagaacgtcactgttacacacgcccaagacatactggaaaagacacacaacgggaaactctgcgatctagatggagtgaagcctctaattttaagagattgtagtgtagctggatggctcctcgggaacccaatgtgtgacgaattcctcaatgtgccggaatggtcttacatagtggagaagatcaatccagccaatgacctctgttacccagggaatttcaacgactatgaagaactgaaacacctattgagcagaataaaccattttgagaaaattcagatcatccccaaaagttcttggtcagatcatgaagcctcagcaggggtgagctcagcatgtccataccagggaaggtcctccttttttagaaatgtggtatggcttatcaaaaaggacaatgcatacccaacaataaagagaagttacaataataccaaccaagaagatcttttggtattgtgggggattcaccatccaaatgatgcggcagagcagacaaggctctatcaaaacccaactacctatatttccgttgggacatcaacactaaaccagagattggtaccaaaaatagccactagatctaaggtaaacgggcaaagtggaaggatggagttcttttggacaattttaaaaccgaatgatgcaataaactttgagagtaatggaaatttcattgctccagaaaatgcatacaaaattgtcaagaaaggggactcaacaattatgaaaagtgagttggaatatggtaactgcaacaccaagtgtcaaactccaataggggcgataaactctagtatgccattccacaacatccaccctctcaccatcggggaatgccccaaatatgtgaaatcaagcagattagtccttgctactgggctcagaaatagccctcaacgagagacacgaggactatttggagctatagcaggttttatagagggaggatggggaatggtagatggttggtatgggtaccaccatagcaacgagcaggggagtgggtacgctgcagacaaagaatccactcaaaaggcaatagatggagtcaccaataaggtcaactcgatcattgacaaaatgaacactcagtttgaggctgttggaagggaatttaataacttagaaaggagaatagaaaatttaaacaagaagatggaagacggattcctagatgtctggacttataatgctgaacttctggttctcatggaaaatgagagaactctagactttcatgactcaaatgtcaagaacctttacgacaaggtccgactacagcttagggataatgcaaaggagcttggtaacggttgtttcgagttctatcacagatgtgataatgaatgtatggaaagtgtaagaaacggaacgtatgactacccgcagtattcagaagaagcaagattaaaaagagaggaaataagtggagtaaaattggaatcaataggaacttaccaaatactgtcaatttattcaacagtggcgagctccctagcactggcaatcatggtggctggtctatctttatggatgtgctccaatggatcgttacaatgcagaatttgcatttaaggcgcgcccacccagcggccgcccgctacgccccaatgatccgaccagcaaaactcgatgtacttccgaggaactgatgtgcataatgcatcaggctggtacattagatccccgcttaccgcgggcaatatagcaacactaaaaactcgatgtacttccgaggaagcgcagtgcataatgctgcgcagtgttgccacataaccactatattaaccatttatctagcggacgccaaaaactcaatgtatttctgaggaagcgtggtgcataatgccacgcagcgtctgcataacttttattatttcttttattaatcaacaaaattttgtttttaacatttcaaaaaaaaaaaaaaaaaaaaaaaaaaaaaaaaaaaa

SEQ ID NO: 32 - DNA, 11884 bp, synthetic construct
  source 1..11884: mol_type = other DNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatggagaaaatagtgcttcttcttgcaatagtcagccttgttaaaagtgatcagatttgcattggttaccatgcaaacaactcgacagagcaggttgacacaataatggaaaagaacgtcactgttacacacgcccaagacatactggaaaagacacacaacgggaaactctgcgatctagatggagtgaagcctctaattttaagagattgtagtgtagctggatggctcctcgggaacccaatgtgtgacgaattcctcaatgtgccggaatggtcttacatagtggagaagatcaatccagccaatgacctctgttacccagggaatttcaacgactatgaagaactgaaacacctattgagcagaataaaccattttgagaaaattcagatcatccccaaaagttcttggtcagatcatgaagcctcagcaggggtgagctcagcatgtccataccagggaaggtcctccttttttagaaatgtggtatggcttatcaaaaaggacaatgcatacccaacaataaagagaagttacaataataccaaccaagaagatcttttggtattgtgggggattcaccatccaaatgatgcggcagagcagacaaggctctatcaaaacccaactacctatatttccgttgggacatcaacactaaaccagagattggtaccaaaaatagccactagatctaaggtaaacgggcaaagtggaaggatggagttcttttggacaattttaaaaccgaatgatgcaataaactttgagagtaatggaaatttcattgctccagaaaatgcatacaaaattgtcaagaaaggggactcaacaattatgaaaagtgagttggaatatggtaactgcaacaccaagtgtcaaactccaataggggcgataaactctagtatgccattccacaacatccaccctctcaccatcggggaatgccccaaatatgtgaaatcaagcagattagtccttgctactgggctcagaaatagccctcaacgagagacacgaggactatttggagctatagcaggttttatagagggaggatggggaatggtagatggttggtatgggtaccaccatagcaacgagcaggggagtgggtacgctgcagacaaagaatccactcaaaaggcaatagatggagtcaccaataaggtcaactcgatcattgacaaaatgaacactcagtttgaggctgttggaagggaatttaataacttagaaaggagaatagaaaatttaaacaagaagatggaagacggattcctagatgtctggacttataatgctgaacttctggttctcatggaaaatgagagaactctagactttcatgactcaaatgtcaagaacctttacgacaaggtccgactacagcttagggataatgcaaaggagcttggtaacggttgtttcgagttctatcacagatgtgataatgaatgtatggaaagtgtaagaaacggaacgtatgactacccgcagtattcagaagaagcaagattaaaaagagaggaaataagtggagtaaaattggaatcaataggaacttaccaaatactgtcaatttattcaacagtggcgagctccctagcactggcaatcatggtggctggtctatctttatggatgtgctccaatggatcgttacaatgcagaatttgcatttaaggcgcgcccacccagcggccgcccgctacgccccaatgatccgaccagcaaaactcgatgtacttccgaggaactgatgtgcataatgcatcaggctggtacattagatccccgcttaccgcgggcaatatagcaacactaaaaactcgatgtacttccgaggaagcgcagtgcataatgctgcgcagtgttgccacataaccactatattaaccatttatctagcggacgccaaaaactcaatgtatttctgaggaagcgtggtgcataatgccacgcagcgtctgcataacttttattatttcttttattaatcaacaaaattttgtttttaacatttcaaaaaaaaaaaaaaaaaaaaaaaaaaaaaaaaaaaaagaagagcgtttaaacacgtgatatctggcctcatgggccttcctttcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaggcagcagattacgcgcagaaaaaaaggatctcaagaagatcctttgatcttttctacggggtctgacgctcagtggaacgaaaactcacgttaagggattttggtcatgaatacacggtgcctgactgcgttagcaatttaactgtgataaactaccgcattaaagcttatcgatgataagctgtcaaacatgagaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacacgcgtaatacgactcactatag

SEQ ID NO: 33 - DNA, 38 bp, synthetic construct
  source 1..38: mol_type = other DNA
taacctgaatggactacgacatagtctagtccgccaag

(19) United States
(12) Patent Application Publication
(10) Pub. No.: US 2026/0306623 A1
(43) Pub. Date: Oct. 8, 2026

(54) Vaccine Adjuvants

(71) Applicant: Seqirus Inc., Holly Springs, NC (US)

(72) Inventors: Douglas G. Widman, Holly Springs, NC (US); Palaniappan Ramanathan, Holly Springs, NC (US)

(73) Assignee: Seqirus Inc., Holly Springs, NC (US)

(21) Appl. No.: 19/482,978

(22) PCT Filed: May 13, 2024
(86) PCT No.: PCT/IB2024/054623

§ 371 Date: Nov. 10, 2025

Related U.S. Application Data
(60) Provisional application No. 63/501,718, filed on May 12, 2023.

(51) Int. Cl. A61K 39/385; A61K 39/00; A61K 39/12; C07K 14/005; C12N 7/00; C12N 15/85

(52) U.S. Cl. CPC A61K 39/385; A61K 39/12; C07K 14/005; C12N 7/00; C12N 15/85; A61K 2039/53; A61K 2039/55555; A61K 2039/6031; C12N 2770/36122; C12N 2770/36134; C12N 2840/203

(57) ABSTRACT

The present disclosure relates to RNAs and other polynucleotides encoding an antigen, which can be used in vaccine compositions. In some examples, the RNA or polynucleotide also encodes an adjuvant, such as cathelicidin or a fragment thereof. The present disclosure also relates to use of the RNAs, polynucleotides or compositions in methods of inducing an immune response in a subject.

### RELATED APPLICATION DATA

The present application claims priority from U.S. Patent Application No. 63/501,718 filed 12 May 2023 entitled “Vaccine Adjuvants”, the entire contents of which is hereby incorporated by reference.

### SEQUENCE LISTING

The present application is filed together with a Sequence Listing in electronic form. The entire contents of the Sequence Listing are hereby incorporated by reference.

### FIELD

The present disclosure relates to RNA and/or protein based adjuvants for use in vaccine compositions.

### BACKGROUND

Bacterial, viral, and parasitic infections are wide spread in humans and animals. Diseases caused by these infectious agents are often resistant to antimicrobial pharmaceutical therapy, leaving no effective means of treatment. Consequently, a vaccinology approach is increasingly used to control infectious disease. A whole infectious pathogen can be made suitable for use in a vaccine formulation after chemical inactivation or appropriate genetic manipulation. Alternatively, a protein subunit of the pathogen can be expressed in a recombinant expression system and purified for use in a vaccine formulation. Vaccines can be made more efficacious by enhancing immunogenicity and/or slowing release of the antigens from the injection site.

Traditional vaccines are generally composed of a crude preparation of inactivated or killed or modified live pathogenic microorganisms. The impurities associated with these cultures of pathological microorganisms may act as an adjuvant to enhance the immune response. However, the immunity invoked by vaccines that use homogeneous preparations of pathological microorganisms or purified protein subunits as antigens is often poor. The addition of certain exogenous materials that act as adjuvants therefore becomes necessary. Further, synthetic and subunit vaccines are expensive to produce, and the addition of an adjuvant potentially permits the use of a smaller dose of antigen to stimulate a similar immune response, thereby reducing the production cost of the vaccine. Thus, the effectiveness of some injectable medicinal agents may be significantly increased when the agent is combined with an adjuvant.

Adjuvants are generally utilised to increase the magnitude or function of the antibody response, increase cell mediated immunity, induce mucosal immunity, or reduce antigen dose. The first adjuvant reported was Freund's Complete Adjuvant (FCA) which contains a water-in-oil emulsion and extracts of mycobacterium. FCA is however poorly tolerated and can cause uncontrolled inflammation. Since the discovery of FCA over 80 years ago, efforts have been made to reduce the unwanted side effects of adjuvants. Some materials used as adjuvants include metallic oxides (e.g., aluminum hydroxide), alum, inorganic chelates of salts, gelatins, various paraffin-type oils, synthesized resins, alginates, mucoid and polysaccharide compounds, caseinates, and blood-derived substances such as fibrin clots. While these materials are generally efficacious at stimulating the immune system, issues have arisen in relation to their suitability for use in vaccines due to adverse effects in the host such as production of abscesses, organ damage, carcinogenicity and allergenic responses. Undesirable pharmaceutical properties including rapid dispersion or poor control of dispersion from the injection site, or swelling of the material have also been reported.

Therefore, a skilled person will understand that there remains a need for the development of new adjuvants for use in the generation of vaccines.

### SUMMARY

The present disclosure is based on the inventors' identification of an adjuvant for use in immunogenic and vaccine compositions. In particular, the findings by the inventors provide basis for a RNA comprising a nucleotide sequence encoding an adjuvant and a nucleotide sequence encoding an antigen of interest, wherein the adjuvant is a cathelicidin or a fragment thereof. The findings by the inventors also provide basis for immunogenic compositions comprising a RNA comprising a nucleotide sequence encoding a cathelicidin or a fragment thereof and a nucleotide sequence encoding an antigen of interest, wherein the composition further comprises a cathelicidin polypeptide. The findings by the inventors also provide basis for immunogenic compositions comprising a RNA comprising a nucleotide sequence encoding an antigen of interest and a cathelicidin polypeptide. Furthermore, the findings by the inventors provide basis for methods of treating or preventing or delaying progression of a disease or disorder in a subject using the immunogenic compositions of the disclosure.

Accordingly, the present disclosure provides a polynucleotide comprising a first nucleotide sequence encoding an antigen operably linked to a regulatory element, and a second nucleotide sequence encoding an adjuvant operably linked to a regulatory element, wherein the adjuvant is a cathelicidin or a fragment thereof.

The present disclosure also provides a RNA comprising a first nucleotide sequence encoding an antigen operably linked to a regulatory element, and a second nucleotide sequence encoding an adjuvant operably linked to a regulatory element, wherein the adjuvant is a cathelicidin or a fragment thereof.

The present disclosure also provides a cRNA comprising a first nucleotide sequence encoding an antigen operably linked to a regulatory element, and a second nucleotide sequence encoding an adjuvant operably linked to a regulatory element, wherein the adjuvant is a cathelicidin or a fragment thereof.

The present disclosure also provides a self-replicating RNA comprising a first nucleotide sequence encoding an antigen operably linked to a regulatory element, and a second nucleotide sequence encoding an adjuvant operably linked to a regulatory element, wherein the adjuvant is a cathelicidin or a fragment thereof.

In an example, the self-replicating RNA comprises in 5′ to 3′ order:
a) the nucleotide sequence encoding an antigen operably linked to a regulatory element; and
b) the nucleotide sequence encoding an adjuvant operably linked to a regulatory element, wherein the adjuvant is a cathelicidin or a fragment thereof.

In another example, the self-replicating RNA comprises in 5′ to 3′ order:
a) the nucleotide sequence encoding an adjuvant operably linked to a regulatory element, wherein the adjuvant is a cathelicidin or a fragment thereof; and
b) the nucleotide sequence encoding an antigen operably linked to a regulatory element.

In an example, the self-replicating RNA comprises in 5′ to 3′ order:
a) the nucleotide sequence encoding an antigen operably linked to a SG promoter; and
b) the nucleotide sequence encoding an adjuvant operably linked to a regulatory element selected from the group consisting of a SG promoter and an internal ribosome entry site (IRES), wherein the adjuvant is a cathelicidin or a fragment thereof.

In another example, the self-replicating RNA comprises in 5′ to 3′ order:
a) the nucleotide sequence encoding an adjuvant operably linked to a regulatory element selected from the group consisting of a SG promoter and an internal ribosome entry site (IRES), wherein the adjuvant is a cathelicidin or a fragment thereof; and
b) the nucleotide sequence encoding an antigen operably linked to a SG promoter.

In an example, the self-replicating RNA comprises in 5′ to 3′ order:
a) the nucleotide sequence encoding an adjuvant operably linked to a SG promoter, wherein the adjuvant is a cathelicidin or a fragment thereof; and
b) the nucleotide sequence encoding an antigen operably linked to a regulatory element selected from the group consisting of a SG promoter and an internal ribosome entry site (IRES).

In an example, the self-replicating RNA comprises in 5′ to 3′ order:
a) the nucleotide sequence encoding an antigen operably linked to a SG promoter; and
b) the nucleotide sequence encoding an adjuvant operably linked to a SG promoter, wherein the adjuvant is a cathelicidin or a fragment thereof.

In an example, the self-replicating RNA comprises in 5′ to 3′ order:
a) the nucleotide sequence encoding an adjuvant operably linked to a SG promoter, wherein the adjuvant is a cathelicidin or a fragment thereof; and
b) the nucleotide sequence encoding an antigen operably linked to a SG promoter.

In an example, the regulatory element is selected from the group consisting of a subgenomic (SG) promoter, an internal ribosome entry site (IRES) and a Kozac consensus sequence or a combination thereof. In an example, the regulatory element is a SG promoter.

In an example, the polynucleotide, RNA, cRNA or self-replicating RNA encoding the adjuvant is operably linked to the same regulatory element as the nucleotide sequence encoding the antigen.

In an example, the self-replicating RNA comprises a second antigen. In another example, the self-replicating RNA comprises second and third antigens.

The present disclosure also provides a polynucleotide comprising: a) a first nucleotide sequence encoding a first polypeptide of interest; and b) a second nucleotide sequence encoding an adjuvant operably linked to a regulatory element selected from the group consisting of a subgenomic (SG) promoter and an internal ribosome entry site (IRES), wherein the adjuvant is a cathelicidin or a fragment thereof.

In one example, the polynucleotide comprises, in order from 5′ to 3′: a) a first nucleotide sequence encoding a first polypeptide of interest; and b) a second nucleotide sequence encoding an adjuvant operably linked to a regulatory element selected from the group consisting of a SG promoter and an IRES, wherein the adjuvant is a cathelicidin or a fragment thereof.

In one example, the polynucleotide comprises, in order from 5′ to 3′: a) a first nucleotide sequence encoding an adjuvant; and b) a second nucleotide sequence encoding a polypeptide of interest operably linked to a regulatory element selected from the group consisting of a SG promoter and an IRES, wherein the adjuvant is a cathelicidin or a fragment thereof.

The present disclosure also provides a polynucleotide comprising: a) a first nucleotide sequence encoding a first antigen of interest; and b) a second nucleotide sequence encoding an adjuvant operably linked to a regulatory element selected from the group consisting of a SG promoter and an IRES, wherein the adjuvant is a cathelicidin or a fragment thereof.

In one example, the polynucleotide comprises, in order from 5′ to 3′: a) a first nucleotide sequence encoding a first antigen of interest; and b) a second nucleotide sequence encoding an adjuvant operably linked to a regulatory element selected from the group consisting of a SG promoter and an IRES, wherein the adjuvant is a cathelicidin or a fragment thereof.

In one example, the polynucleotide comprises, in order from 5′ to 3′: a) a first nucleotide sequence encoding an adjuvant; and b) a second nucleotide sequence encoding an antigen of interest operably linked to a regulatory element selected from the group consisting of a SG promoter and an IRES, wherein the adjuvant is a cathelicidin or a fragment thereof.

In one example, the polynucleotide comprises, in order from 5′ to 3′: a) a first nucleotide sequence encoding a first antigen of interest operably linked to a SG promoter; and b) a second nucleotide sequence encoding an adjuvant operably linked to a SG promoter, wherein the adjuvant is a cathelicidin or a fragment thereof.

In one example, the polynucleotide comprises, in order from 5′ to 3′: a) a first nucleotide sequence encoding an adjuvant operably linked to a SG promoter; and b) a second nucleotide sequence encoding an antigen of interest operably linked to a SG promoter, wherein the adjuvant is a cathelicidin or a fragment thereof.

In an example, the polynucleotide is RNA or DNA. In one example, the RNA is messenger RNA (mRNA). In another example, the mRNA is conventional mRNA (cRNA) or self-replicating RNA.

Accordingly, the present disclosure provides a RNA comprising: a) a first nucleotide sequence encoding a first antigen of interest; and b) a second nucleotide sequence encoding an adjuvant operably linked to a regulatory element selected from the group consisting of a SG promoter and an IRES, wherein the adjuvant is a cathelicidin or a fragment thereof.

In one example, the RNA comprises, in order from 5′ to 3′: a) a first nucleotide sequence encoding a first antigen of interest; and b) a second nucleotide sequence encoding an adjuvant operably linked to a regulatory element selected from the group consisting of a SG promoter and an IRES, wherein the adjuvant is a cathelicidin or a fragment thereof.

In one example, the RNA comprises, in order from 5′ to 3′: a) a first nucleotide sequence encoding an adjuvant; and b) a second nucleotide sequence encoding an antigen of interest operably linked to a regulatory element selected from the group consisting of a SG promoter and an IRES, wherein the adjuvant is a cathelicidin or a fragment thereof.

In one example, the RNA comprises, in order from 5′ to 3′: a) a first nucleotide sequence encoding a first antigen of interest operably linked to a SG promoter; and b) a second nucleotide sequence encoding an adjuvant operably linked to a SG promoter, wherein the adjuvant is a cathelicidin or a fragment thereof.

In one example, the RNA comprises, in order from 5′ to 3′: a) a first nucleotide sequence encoding an adjuvant operably linked to a SG promoter; and b) a second nucleotide sequence encoding an antigen of interest operably linked to a SG promoter, wherein the adjuvant is a cathelicidin or a fragment thereof.

The present disclosure also provides a cRNA comprising: a) a first nucleotide sequence encoding a first antigen of interest; and b) a second nucleotide sequence encoding an adjuvant operably linked to a regulatory element selected from the group consisting of a SG promoter and an IRES, wherein the adjuvant is a cathelicidin or a fragment thereof.

In one example, the cRNA comprises, in order from 5′ to 3′: a) a first nucleotide sequence encoding a first antigen of interest; and b) a second nucleotide sequence encoding an adjuvant operably linked to a regulatory element selected from the group consisting of a SG promoter and an IRES, wherein the adjuvant is a cathelicidin or a fragment thereof.

In one example, the cRNA comprises, in order from 5′ to 3′: a) a first nucleotide sequence encoding an adjuvant; and b) a second nucleotide sequence encoding an antigen of interest operably linked to a regulatory element selected from the group consisting of a SG promoter and an IRES, wherein the adjuvant is a cathelicidin or a fragment thereof.

In one example, the cRNA comprises, in order from 5′ to 3′: a) a first nucleotide sequence encoding a first antigen of interest operably linked to a SG promoter; and b) a second nucleotide sequence encoding an adjuvant operably linked to a SG promoter, wherein the adjuvant is a cathelicidin or a fragment thereof.

In one example, the cRNA comprises, in order from 5′ to 3′: a) a first nucleotide sequence encoding an adjuvant operably linked to a SG promoter; and b) a second nucleotide sequence encoding an antigen of interest operably linked to a SG promoter, wherein the adjuvant is a cathelicidin or a fragment thereof.

The present disclosure also provides a composition comprising a cRNA encoding a first antigen of interest and a cRNA encoding an adjuvant, wherein the adjuvant is a cathelicidin or a fragment thereof.

The present disclosure further provides a self-replicating RNA comprising: a) a first nucleotide sequence encoding a first antigen of interest; and b) a second nucleotide sequence encoding an adjuvant operably linked to a regulatory element selected from the group consisting of a SG promoter and an IRES, wherein the adjuvant is a cathelicidin or a fragment thereof.

In one example, the self-replicating RNA comprises, in order from 5′ to 3′: a) a first nucleotide sequence encoding a first antigen of interest; and b) a second nucleotide sequence encoding an adjuvant operably linked to a regulatory element selected from the group consisting of a SG promoter and an IRES, wherein the adjuvant is a cathelicidin or a fragment thereof.

In one example, the self-replicating RNA comprises, in order from 5′ to 3′: a) a first nucleotide sequence encoding an adjuvant; and b) a second nucleotide sequence encoding an antigen of interest operably linked to a regulatory element selected from the group consisting of a SG promoter and an IRES, wherein the adjuvant is a cathelicidin or a fragment thereof.

In one example, the self-replicating RNA comprises, in order from 5′ to 3′: a) a first nucleotide sequence encoding a first antigen of interest operably linked to a SG promoter; and b) a second nucleotide sequence encoding an adjuvant operably linked to a SG promoter, wherein the adjuvant is a cathelicidin or a fragment thereof.

In one example, the self-replicating RNA comprises, in order from 5′ to 3′: a) a first nucleotide sequence encoding an adjuvant operably linked to a SG promoter; and b) a second nucleotide sequence encoding an antigen of interest operably linked to a SG promoter, wherein the adjuvant is a cathelicidin or a fragment thereof.

In one example, the first nucleotide sequence is operably linked to a regulatory element. In another example, the regulatory element is operably linked to the 5′ end of the first nucleotide sequence. In one example, the regulatory element is selected from the group consisting of a Kozak consensus sequence, an IRES, a SG promoter and combinations thereof. For example, the regulatory element is a Kozak consensus sequence. In another example, the regulatory element is an IRES. In an example, the nucleotide sequence encoding an adjuvant is operably linked to an IRES located 3′ to the nucleotide sequence encoding the adjuvant. In another example, the regulatory element is a SG promoter.

In one example, the Kozak consensus sequence comprises or consists of a sequence set forth in SEQ ID NO: 19. In one example, the Kozak consensus sequence consists of a sequence set forth in SEQ ID NO: 19 (GCCACC). In one example, the Kozak consensus sequence comprises a sequence set forth in SEQ ID NO: 19 (GCCACC). For example, the Kozak consensus sequence is ACCATGG.

In one example, the Kozak consensus sequence comprises or consists of a sequence set forth in SEQ ID NO: 20 (ACCATGG). In one example, the Kozak consensus sequence consists of a sequence set forth in SEQ ID NO: 20 (ACCATGG). In one example, the Kozak consensus sequence comprises a sequence set forth in SEQ ID NO: 20 (ACCATGG).

The present disclosure provides a polynucleotide comprising: a) a first nucleotide sequence encoding a first antigen of interest operably linked to a regulatory element selected from the group consisting of a Kozak consensus sequence, an IRES, a SG promoter and combinations thereof; and b) a second nucleotide sequence encoding an adjuvant operably linked to a regulatory element selected from the group consisting of a SG promoter and an IRES, wherein the adjuvant is a cathelicidin or a fragment thereof.

In one example, the polynucleotide comprises, in order from 5′ to 3′: a) a first nucleotide sequence encoding a first antigen of interest operably linked to a regulatory element selected from the group consisting of a Kozak consensus sequence, an IRES, a SG promoter and combinations thereof; and b) a second nucleotide sequence encoding an adjuvant operably linked to a regulatory element selected from the group consisting of a SG promoter and an IRES, wherein the adjuvant is a cathelicidin or a fragment thereof.

In one example, the polynucleotide comprises, in order from 5′ to 3′: a) a first nucleotide sequence encoding an adjuvant operably linked to a regulatory element selected from the group consisting of a Kozak consensus sequence, an IRES, a SG promoter and combinations thereof; and b) a second nucleotide sequence encoding an antigen of interest operably linked to a regulatory element selected from the group consisting of a SG promoter and an IRES, wherein the adjuvant is a cathelicidin or a fragment thereof.

The present disclosure provides a RNA comprising: a) a first nucleotide sequence encoding a first antigen of interest operably linked to a regulatory element selected from the group consisting of a Kozak consensus sequence, an IRES, a SG promoter and combinations thereof; and b) a second nucleotide sequence encoding an adjuvant operably linked to a regulatory element selected from the group consisting of a SG promoter and an IRES, wherein the adjuvant is a cathelicidin or a fragment thereof.

In one example, the RNA comprises, in order from 5′ to 3′: a) a first nucleotide sequence encoding a first antigen of interest operably linked to a regulatory element selected from the group consisting of a Kozak consensus sequence, an IRES, a SG promoter and combinations thereof; and b) a second nucleotide sequence encoding an adjuvant operably linked to a regulatory element selected from the group consisting of a SG promoter and an IRES, wherein the adjuvant is a cathelicidin or a fragment thereof.

In one example, the RNA comprises, in order from 5′ to 3′: a) a first nucleotide sequence encoding an adjuvant operably linked to a regulatory element selected from the group consisting of a Kozak consensus sequence, an IRES, a SG promoter and combinations thereof; and b) a second nucleotide sequence encoding an antigen of interest operably linked to a regulatory element selected from the group consisting of a SG promoter and an IRES, wherein the adjuvant is a cathelicidin or a fragment thereof.

The present disclosure provides a cRNA comprising: a) a first nucleotide sequence encoding a first antigen of interest operably linked to a regulatory element selected from the group consisting of a Kozak consensus sequence, an IRES, a SG promoter and combinations thereof; and b) a second nucleotide sequence encoding an adjuvant operably linked to a regulatory element selected from the group consisting of a SG promoter and an IRES, wherein the adjuvant is a cathelicidin or a fragment thereof.

In one example, the cRNA comprises, in order from 5′ to 3′: a) a first nucleotide sequence encoding a first antigen of interest operably linked to a regulatory element selected from the group consisting of a Kozak consensus sequence, an IRES, a SG promoter and combinations thereof; and b) a second nucleotide sequence encoding an adjuvant operably linked to a regulatory element selected from the group consisting of a SG promoter and an IRES, wherein the adjuvant is a cathelicidin or a fragment thereof.

In one example, the cRNA comprises, in order from 5′ to 3′: a) a first nucleotide sequence encoding an adjuvant operably linked to a regulatory element selected from the group consisting of a Kozak consensus sequence, an IRES, a SG promoter and combinations thereof; and b) a second nucleotide sequence encoding an antigen of interest operably linked to a regulatory element selected from the group consisting of a SG promoter and an IRES, wherein the adjuvant is a cathelicidin or a fragment thereof.

The present disclosure provides a self-replicating RNA comprising: a) a first nucleotide sequence encoding a first antigen of interest operably linked to a regulatory element selected from the group consisting of a Kozak consensus sequence, an IRES, a SG promoter and combinations thereof; and b) a second nucleotide sequence encoding an adjuvant operably linked to a regulatory element selected from the group consisting of a SG promoter and an IRES, wherein the adjuvant is a cathelicidin or a fragment thereof.

In one example, the self-replicating RNA comprises, in order from 5′ to 3′: a) a first nucleotide sequence encoding a first antigen of interest operably linked to a regulatory element selected from the group consisting of a Kozak consensus sequence, an IRES, a SG promoter and combinations thereof; and b) a second nucleotide sequence encoding an adjuvant operably linked to a regulatory element selected from the group consisting of a SG promoter and an IRES, wherein the adjuvant is a cathelicidin or a fragment thereof.

In one example, the self-replicating RNA comprises, in order from 5′ to 3′: a) a first nucleotide sequence encoding an adjuvant operably linked to a regulatory element selected from the group consisting of a Kozak consensus sequence, an IRES, a SG promoter and combinations thereof; and b) a second nucleotide sequence encoding an antigen of interest operably linked to a regulatory element selected from the group consisting of a SG promoter and an IRES, wherein the adjuvant is a cathelicidin or a fragment thereof.

In one example, the first nucleotide sequence is operably linked to a Kozak consensus sequence.

In one example, the first nucleotide sequence is operably linked to a Kozak consensus sequence and a SG promoter. For example, the Kozak consensus sequence is operably linked to the 5′ end of the SG promoter which is operably linked to the 5′ end of the first nucleotide sequence.

In one example, the first nucleotide sequence is operably linked to a Kozak consensus sequence and an IRES. For example, the Kozak consensus sequence is operably linked to the 5′ end of the IRES which is operably linked to the 5′ end of the first nucleotide sequence.

In one example, the first nucleotide sequence is operably linked to a SG promoter. In some examples, first nucleotide sequence is operably linked to a SG promoter and the second nucleotide sequence is operably linked to a SG promoter.

In one example, the first nucleotide sequence is operably linked to an IRES.

The present disclosure provides a polynucleotide comprising: a) a first nucleotide sequence encoding a first antigen of interest operably linked to a Kozak consensus sequence; and b) a second nucleotide sequence encoding an adjuvant operably linked to a regulatory element selected from the group consisting of a SG promoter and an IRES, wherein the adjuvant is a cathelicidin or a fragment thereof.

In one example, the polynucleotide comprises, in order from 5′ to 3′: a) a first nucleotide sequence encoding a first antigen of interest operably linked to a Kozak consensus sequence; and b) a second nucleotide sequence encoding an adjuvant operably linked to a regulatory element selected from the group consisting of a SG promoter and an IRES, wherein the adjuvant is a cathelicidin or a fragment thereof.

In one example, the polynucleotide comprises, in order from 5′ to 3′: a) a first nucleotide sequence encoding an adjuvant operably linked to a Kozak consensus sequence; and b) a second nucleotide sequence encoding an antigen of interest operably linked to a regulatory element selected from the group consisting of a SG promoter and an IRES, wherein the adjuvant is a cathelicidin or a fragment thereof.

The present disclosure provides a polynucleotide comprising: a) a first nucleotide sequence encoding a first antigen of interest operably linked to a Kozak consensus sequence and a SG promoter; and b) a second nucleotide sequence encoding an adjuvant operably linked to a regulatory element selected from the group consisting of a SG promoter and an IRES, wherein the adjuvant is a cathelicidin or a fragment thereof.

In one example, the polynucleotide comprises, in order from 5′ to 3′: a) a first nucleotide sequence encoding a first antigen of interest operably linked to a Kozak consensus sequence and a SG promoter; and b) a second nucleotide sequence encoding an adjuvant operably linked to a regulatory element selected from the group consisting of a SG promoter and an IRES, wherein the adjuvant is a cathelicidin or a fragment thereof.

In one example, the polynucleotide comprises, in order from 5′ to 3′: a) a first nucleotide sequence encoding an adjuvant operably linked to a Kozak consensus sequence and a SG promoter; and b) a second nucleotide sequence encoding an antigen of interest operably linked to a regulatory element selected from the group consisting of a SG promoter and an IRES, wherein the adjuvant is a cathelicidin or a fragment thereof.

The present disclosure provides a polynucleotide comprising: a) a first nucleotide sequence encoding a first antigen of interest operably linked to a Kozak consensus sequence and an IRES; and b) a second nucleotide sequence encoding an adjuvant operably linked to a regulatory element selected from the group consisting of a SG promoter and an IRES, wherein the adjuvant is a cathelicidin or a fragment thereof.

In one example, the polynucleotide comprises, in order from 5′ to 3′: a) a first nucleotide sequence encoding a first antigen of interest operably linked to a Kozak consensus sequence and an IRES; and b) a second nucleotide sequence encoding an adjuvant operably linked to a regulatory element selected from the group consisting of a SG promoter and an IRES, wherein the adjuvant is a cathelicidin or a fragment thereof.

In one example, the polynucleotide comprises, in order from 5′ to 3′: a) a first nucleotide sequence encoding an adjuvant operably linked to a Kozak consensus sequence and an IRES; and b) a second nucleotide sequence encoding an antigen of interest operably linked to a regulatory element selected from the group consisting of a SG promoter and an IRES, wherein the adjuvant is a cathelicidin or a fragment thereof.

The present disclosure provides a polynucleotide comprising: a) a first nucleotide sequence encoding a first antigen of interest operably linked to a SG promoter; and b) a second nucleotide sequence encoding an adjuvant operably linked to a regulatory element selected from the group consisting of a SG promoter and an IRES, wherein the adjuvant is a cathelicidin or a fragment thereof.

In one example, the polynucleotide comprises, in order from 5′ to 3′: a) a first nucleotide sequence encoding a first antigen of interest operably linked to a SG promoter; and b) a second nucleotide sequence encoding an adjuvant operably linked to a regulatory element selected from the group consisting of a SG promoter and an IRES, wherein the adjuvant is a cathelicidin or a fragment thereof.

In one example, the polynucleotide comprises, in order from 5′ to 3′: a) a first nucleotide sequence encoding an adjuvant operably linked to a SG promoter; and b) a second nucleotide sequence encoding an antigen of interest operably linked to a regulatory element selected from the group consisting of a SG promoter and an IRES, wherein the adjuvant is a cathelicidin or a fragment thereof.

The present disclosure provides a polynucleotide comprising: a) a first nucleotide sequence encoding a first antigen of interest operably linked to an IRES; and b) a second nucleotide sequence encoding an adjuvant operably linked to a regulatory element selected from the group consisting of a SG promoter and an IRES, wherein the adjuvant is a cathelicidin or a fragment thereof.

In one example, the polynucleotide comprises, in order from 5′ to 3′: a) a first nucleotide sequence encoding a first antigen of interest operably linked to an IRES; and b) a second nucleotide sequence encoding an adjuvant operably linked to a regulatory element selected from the group consisting of a SG promoter and an IRES, wherein the adjuvant is a cathelicidin or a fragment thereof.

In one example, the polynucleotide comprises, in order from 5′ to 3′: a) a first nucleotide sequence encoding an adjuvant operably linked to an IRES; and b) a second nucleotide sequence encoding an antigen of interest operably linked to a regulatory element selected from the group consisting of a SG promoter and an IRES, wherein the adjuvant is a cathelicidin or a fragment thereof.

The present disclosure provides a RNA comprising: a) a first nucleotide sequence encoding a first antigen of interest operably linked to a Kozak consensus sequence; and b) a second nucleotide sequence encoding an adjuvant operably linked to a regulatory element selected from the group consisting of a SG promoter and an IRES, wherein the adjuvant is a cathelicidin or a fragment thereof.

In one example, the RNA comprises, in order from 5′ to 3′: a) a first nucleotide sequence encoding a first antigen of interest operably linked to a Kozak consensus sequence; and b) a second nucleotide sequence encoding an adjuvant operably linked to a regulatory element selected from the group consisting of a SG promoter and an IRES, wherein the adjuvant is a cathelicidin or a fragment thereof.

In one example, the RNA comprises, in order from 5′ to 3′: a) a first nucleotide sequence encoding an adjuvant operably linked to a Kozak consensus sequence; and b) a second nucleotide sequence encoding an antigen of interest operably linked to a regulatory element selected from the group consisting of a SG promoter and an IRES, wherein the adjuvant is a cathelicidin or a fragment thereof.

The present disclosure provides a RNA comprising: a) a first nucleotide sequence encoding a first antigen of interest operably linked to a Kozak consensus sequence and a SG promoter; and b) a second nucleotide sequence encoding an adjuvant operably linked to a regulatory element selected from the group consisting of a SG promoter and an IRES, wherein the adjuvant is a cathelicidin or a fragment thereof.

In one example, the RNA comprises, in order from 5′ to 3′: a) a first nucleotide sequence encoding a first antigen of interest operably linked to a Kozak consensus sequence and a SG promoter; and b) a second nucleotide sequence encoding an adjuvant operably linked to a regulatory element selected from the group consisting of a SG promoter and an IRES, wherein the adjuvant is a cathelicidin or a fragment thereof.

In one example, the RNA comprises, in order from 5′ to 3′: a) a first nucleotide sequence encoding an adjuvant operably linked to a Kozak consensus sequence and a SG promoter; and b) a second nucleotide sequence encoding an antigen of interest operably linked to a regulatory element selected from the group consisting of a SG promoter and an IRES, wherein the adjuvant is a cathelicidin or a fragment thereof.

The present disclosure provides a RNA comprising: a) a first nucleotide sequence encoding a first antigen of interest operably linked to a Kozak consensus sequence and an IRES; and b) a second nucleotide sequence encoding an adjuvant operably linked to a regulatory element selected from the group consisting of a SG promoter and an IRES, wherein the adjuvant is a cathelicidin or a fragment thereof.

In one example, the RNA comprises, in order from 5′ to 3′: a) a first nucleotide sequence encoding a first antigen of interest operably linked to a Kozak consensus sequence and an IRES; and b) a second nucleotide sequence encoding an adjuvant operably linked to a regulatory element selected from the group consisting of a SG promoter and an IRES, wherein the adjuvant is a cathelicidin or a fragment thereof.

In one example, the RNA comprises, in order from 5′ to 3′: a) a first nucleotide sequence encoding an adjuvant operably linked to a Kozak consensus sequence and an IRES; and b) a second nucleotide sequence encoding an antigen of interest operably linked to a regulatory element selected from the group consisting of a SG promoter and an IRES, wherein the adjuvant is a cathelicidin or a fragment thereof.

The present disclosure provides a RNA comprising: a) a first nucleotide sequence encoding a first antigen of interest operably linked to a SG promoter; and b) a second nucleotide sequence encoding an adjuvant operably linked to a regulatory element selected from the group consisting of a SG promoter and an IRES, wherein the adjuvant is a cathelicidin or a fragment thereof.

In one example, the RNA comprises, in order from 5′ to 3′: a) a first nucleotide sequence encoding a first antigen of interest operably linked to a SG promoter; and b) a second nucleotide sequence encoding an adjuvant operably linked to a regulatory element selected from the group consisting of a SG promoter and an IRES, wherein the adjuvant is a cathelicidin or a fragment thereof.

In one example, the RNA comprises, in order from 5′ to 3′: a) a first nucleotide sequence encoding an adjuvant operably linked to a SG promoter; and b) a second nucleotide sequence encoding an antigen of interest operably linked to a regulatory element selected from the group consisting of a SG promoter and an IRES, wherein the adjuvant is a cathelicidin or a fragment thereof.

The present disclosure provides a RNA comprising: a) a first nucleotide sequence encoding a first antigen of interest operably linked to an IRES; and b) a second nucleotide sequence encoding an adjuvant operably linked to a regulatory element selected from the group consisting of a SG promoter and an IRES, wherein the adjuvant is a cathelicidin or a fragment thereof.

In one example, the RNA comprises, in order from 5′ to 3′: a) a first nucleotide sequence encoding a first antigen of interest operably linked to an IRES; and b) a second nucleotide sequence encoding an adjuvant operably linked to a regulatory element selected from the group consisting of a SG promoter and an IRES, wherein the adjuvant is a cathelicidin or a fragment thereof.

In one example, the RNA comprises, in order from 5′ to 3′: a) a first nucleotide sequence encoding an adjuvant operably linked to an IRES; and b) a second nucleotide sequence encoding an antigen of interest operably linked to a regulatory element selected from the group consisting of a SG promoter and an IRES, wherein the adjuvant is a cathelicidin or a fragment thereof.

The present disclosure provides a cRNA comprising: a) a first nucleotide sequence encoding a first antigen of interest operably linked to a Kozak consensus sequence; and b) a second nucleotide sequence encoding an adjuvant operably linked to a regulatory element selected from the group consisting of a SG promoter and an IRES, wherein the adjuvant is a cathelicidin or a fragment thereof.

In one example, the cRNA comprises, in order from 5′ to 3′: a) a first nucleotide sequence encoding a first antigen of interest operably linked to a Kozak consensus sequence; and b) a second nucleotide sequence encoding an adjuvant operably linked to a regulatory element selected from the group consisting of a SG promoter and an IRES, wherein the adjuvant is a cathelicidin or a fragment thereof.

In one example, the cRNA comprises, in order from 5′ to 3′: a) a first nucleotide sequence encoding an adjuvant operably linked to a Kozak consensus sequence; and b) a second nucleotide sequence encoding an antigen of interest operably linked to a regulatory element selected from the group consisting of a SG promoter and an IRES, wherein the adjuvant is a cathelicidin or a fragment thereof.

The present disclosure provides a cRNA comprising: a) a first nucleotide sequence encoding a first antigen of interest operably linked to a Kozak consensus sequence and a SG promoter; and b) a second nucleotide sequence encoding an adjuvant operably linked to a regulatory element selected from the group consisting of a SG promoter and an IRES, wherein the adjuvant is a cathelicidin or a fragment thereof.

In one example, the cRNA comprises, in order from 5′ to 3′: a) a first nucleotide sequence encoding a first antigen of interest operably linked to a Kozak consensus sequence and a SG promoter; and b) a second nucleotide sequence encoding an adjuvant operably linked to a regulatory element selected from the group consisting of a SG promoter and an IRES, wherein the adjuvant is a cathelicidin or a fragment thereof.

In one example, the cRNA comprises, in order from 5′ to 3′: a) a first nucleotide sequence encoding an adjuvant operably linked to a Kozak consensus sequence and a SG promoter; and b) a second nucleotide sequence encoding an antigen of interest operably linked to a regulatory element selected from the group consisting of a SG promoter and an IRES, wherein the adjuvant is a cathelicidin or a fragment thereof.

The present disclosure provides a cRNA comprising: a) a first nucleotide sequence encoding a first antigen of interest operably linked to a Kozak consensus sequence and an IRES; and b) a second nucleotide sequence encoding an adjuvant operably linked to a regulatory element selected from the group consisting of a SG promoter and an IRES, wherein the adjuvant is a cathelicidin or a fragment thereof.

In one example, the cRNA comprises, in order from 5′ to 3′: a) a first nucleotide sequence encoding a first antigen of interest operably linked to a Kozak consensus sequence and an IRES; and b) a second nucleotide sequence encoding adjuvant operably linked to a regulatory element selected from the group consisting of a SG promoter and an IRES, wherein the adjuvant is a cathelicidin or a fragment thereof.

In one example, the cRNA comprises, in order from 5′ to 3′: a) a first nucleotide sequence encoding an adjuvant operably linked to a Kozak consensus sequence and an IRES; and b) a second nucleotide sequence encoding an antigen of interest operably linked to a regulatory element selected from the group consisting of a SG promoter and an IRES, wherein the adjuvant is a cathelicidin or a fragment thereof.

The present disclosure provides a cRNA comprising: a) a first nucleotide sequence encoding a first antigen of interest operably linked to a SG promoter; and b) a second nucleotide sequence encoding an adjuvant operably linked to a regulatory element selected from the group consisting of a SG promoter and an IRES, wherein the adjuvant is a cathelicidin or a fragment thereof.

In one example, the cRNA comprises, in order from 5′ to 3′: a) a first nucleotide sequence encoding a first antigen of interest operably linked to a SG promoter; and b) a second nucleotide sequence encoding an adjuvant operably linked to a regulatory element selected from the group consisting of a SG promoter and an IRES, wherein the adjuvant is a cathelicidin or a fragment thereof.

In one example, the cRNA comprises, in order from 5′ to 3′: a) a first nucleotide sequence encoding an adjuvant operably linked to a SG promoter; and b) a second nucleotide sequence encoding an antigen of interest operably linked to a regulatory element selected from the group consisting of a SG promoter and an IRES, wherein the adjuvant is a cathelicidin or a fragment thereof.

The present disclosure provides a cRNA comprising: a) a first nucleotide sequence encoding a first antigen of interest operably linked to an IRES; and b) a second nucleotide sequence encoding an adjuvant operably linked to a regulatory element selected from the group consisting of a SG promoter and an IRES, wherein the adjuvant is a cathelicidin or a fragment thereof.

In one example, the cRNA comprises, in order from 5′ to 3′: a) a first nucleotide sequence encoding a first antigen of interest operably linked to an IRES; and b) a second nucleotide sequence encoding an adjuvant operably linked to a regulatory element selected from the group consisting of a SG promoter and an IRES, wherein the adjuvant is a cathelicidin or a fragment thereof.

In one example, the cRNA comprises, in order from 5′ to 3′: a) a first nucleotide sequence encoding an adjuvant operably linked to an IRES; and b) a second nucleotide sequence encoding an antigen of interest operably linked to a regulatory element selected from the group consisting of a SG promoter and an IRES, wherein the adjuvant is a cathelicidin or a fragment thereof.

The present disclosure provides a self-replicating RNA comprising: a) a first nucleotide sequence encoding a first antigen of interest operably linked to a Kozak consensus sequence; and b) a second nucleotide sequence encoding an adjuvant operably linked to a regulatory element selected from the group consisting of a SG promoter and an IRES, wherein the adjuvant is a cathelicidin or a fragment thereof.

In one example, the self-replicating RNA comprises, in order from 5′ to 3′: a) a first nucleotide sequence encoding a first antigen of interest operably linked to a Kozak consensus sequence; and b) a second nucleotide sequence encoding an adjuvant operably linked to a regulatory element selected from the group consisting of a SG promoter and an IRES, wherein the adjuvant is a cathelicidin or a fragment thereof.

In one example, the self-replicating RNA comprises, in order from 5′ to 3′: a) a first nucleotide sequence encoding an adjuvant operably linked to a Kozak consensus sequence; and b) a second nucleotide sequence encoding an antigen of interest operably linked to a regulatory element selected from the group consisting of a SG promoter and an IRES, wherein the adjuvant is a cathelicidin or a fragment thereof.

The present disclosure provides a self-replicating RNA comprising: a) a first nucleotide sequence encoding a first antigen of interest operably linked to a Kozak consensus sequence and a SG promoter; and b) a second nucleotide sequence encoding an adjuvant operably linked to a regulatory element selected from the group consisting of a SG promoter and an IRES, wherein the adjuvant is a cathelicidin or a fragment thereof.

In one example, the self-replicating RNA comprises, in order from 5′ to 3′: a) a first nucleotide sequence encoding a first antigen of interest operably linked to a Kozak consensus sequence and a SG promoter; and b) a second nucleotide sequence encoding an adjuvant operably linked to a regulatory element selected from the group consisting of a SG promoter and an IRES, wherein the adjuvant is a cathelicidin or a fragment thereof.

In one example, the self-replicating RNA comprises, in order from 5′ to 3′: a) a first nucleotide sequence encoding an adjuvant operably linked to a Kozak consensus sequence and a SG promoter; and b) a second nucleotide sequence encoding an antigen of interest operably linked to a regulatory element selected from the group consisting of a SG promoter and an IRES, wherein the adjuvant is a cathelicidin or a fragment thereof.

The present disclosure provides a self-replicating RNA comprising: a) a first nucleotide sequence encoding a first antigen of interest operably linked to a Kozak consensus sequence and an IRES; and b) a second nucleotide sequence encoding an adjuvant operably linked to a regulatory element selected from the group consisting of a SG promoter and an IRES, wherein the adjuvant is a cathelicidin or a fragment thereof.

In one example, the self-replicating RNA comprises, in order from 5′ to 3′: a) a first nucleotide sequence encoding a first antigen of interest operably linked to a Kozak consensus sequence and an IRES; and b) a second nucleotide sequence encoding an adjuvant operably linked to a regulatory element selected from the group consisting of a SG promoter and an IRES, wherein the adjuvant is a cathelicidin or a fragment thereof.

In one example, the self-replicating RNA comprises, in order from 5′ to 3′: a) a first nucleotide sequence encoding an adjuvant operably linked to a Kozak consensus sequence and an IRES; and b) a second nucleotide sequence encoding an antigen of interest operably linked to a regulatory element selected from the group consisting of a SG promoter and an IRES, wherein the adjuvant is a cathelicidin or a fragment thereof.

The present disclosure provides a self-replicating RNA comprising: a) a first nucleotide sequence encoding a first antigen of interest operably linked to a SG promoter; and b) a second nucleotide sequence encoding an adjuvant operably linked to a regulatory element selected from the group consisting of a SG promoter and an IRES, wherein the adjuvant is a cathelicidin or a fragment thereof.

In one example, the self-replicating RNA comprises, in order from 5′ to 3′: a) a first nucleotide sequence encoding a first antigen of interest operably linked to a SG promoter; and b) a second nucleotide sequence encoding an adjuvant operably linked to a regulatory element selected from the group consisting of a SG promoter and an IRES, wherein the adjuvant is a cathelicidin or a fragment thereof.

In one example, the self-replicating RNA comprises, in order from 5′ to 3′: a) a first nucleotide sequence encoding an adjuvant operably linked to a SG promoter; and b) a second nucleotide sequence encoding an antigen of interest operably linked to a regulatory element selected from the group consisting of a SG promoter and an IRES, wherein the adjuvant is a cathelicidin or a fragment thereof.

The present disclosure provides a self-replicating RNA comprising: a) a first nucleotide sequence encoding a first antigen of interest operably linked to an IRES; and b) a second nucleotide sequence encoding an adjuvant operably linked to a regulatory element selected from the group consisting of a SG promoter and an IRES, wherein the adjuvant is a cathelicidin or a fragment thereof.

In one example, the self-replicating RNA comprises, in order from 5′ to 3′: a) a first nucleotide sequence encoding a first antigen of interest operably linked to an IRES; and b) a second nucleotide sequence encoding an adjuvant operably linked to a regulatory element selected from the group consisting of a SG promoter and an IRES, wherein the adjuvant is a cathelicidin or a fragment thereof.

In one example, the self-replicating RNA comprises, in order from 5′ to 3′: a) a first nucleotide sequence encoding an adjuvant operably linked to an IRES; and b) a second nucleotide sequence encoding an antigen of interest operably linked to a regulatory element selected from the group consisting of a SG promoter and an IRES, wherein the adjuvant is a cathelicidin or a fragment thereof.

In one example, the polynucleotide is a bicistronic RNA. For example, the polynucleotide is a bicistronic cRNA. In one example, the cRNA is a bicistronic cRNA. In another example, the polynucleotide is a bicistronic self-replicating mRNA. For example, the self-replicating RNA is a bicistronic self-replicating RNA.

In one example, the second nucleotide sequence is operably linked to an IRES.

In one example, the second nucleotide sequence is operably linked to a SG promoter.

In one example, the polynucleotide is a multicistronic RNA. For example, the polynucleotide is a multicistronic cRNA. For example, the cRNA is a multicistronic cRNA. In another example, the polynucleotide is a multicistronic self-replicating mRNA. For example, the self-replicating RNA is a multicistronic self-replicating mRNA.

In one example, the SG promoter is a native SG promoter. For example, a native SG promoter is a promoter that is native to the RNA virus from which it is derived and/or based on (e.g., an alphavirus). In one example, the native SG promoter is a native alphavirus SG promoter.

In one example, the SG promoter is a minimal SG promoter or an extended SG promoter.

In one example, the SG promoter is a minimal SG promoter. In one example, the native SG promoter is a minimal SG promoter. For example, the minimal SG promoter is the minimal sequence required for initiation of transcription. In one example, the minimal native SG promoter is 49 nucleotides in length. In one example, the minimal SG promoter is 49 nucleotides in length. In one example, the minimal native SG promoter is 38 nucleotides in length. In one example, the minimal SG promoter is 38 nucleotides in length. In one example, the minimal native SG promoter is encoded by a sequence comprising or consisting of a sequence set forth in SEQ ID NO: 14. In one example, the minimal SG promoter is encoded by a sequence comprising or consisting of a sequence set forth in SEQ ID NO: 14. In one example, the minimal native SG promoter is encoded by a sequence comprising or consisting of a sequence set forth in SEQ ID NO: 38. In one example, the minimal SG promoter is encoded by a sequence comprising or consisting of a sequence set forth in SEQ ID NO: 33.

In some examples, the minimal SG promoter is encoded by a sequence set forth in SEQ ID NO: 14 or 33. In some examples, the minimal SG promoter is encoded by a sequence set forth in SEQ ID NO: 14.

In one example, the SG promoter is an extended SG promoter. In one example, the native SG promoter is an extended SG promoter. For example, the extended SG promoter is extended at the 5′ end with nucleotides occurring in a sequence encoding a non-structural protein (e.g., NSP4) of the RNA virus (e.g., an alphavirus). In one example, the extended SG promoter is extended at the 5′ end with nucleotides occurring in a sequence encoding an alphavirus NSP4. The addition of nucleotides to the 5′ end of the SG promoter sequence did not interfere with expression of the non-structural protein and viral replicase, e.g., alphavirus NSP4.

In one example, the SG promoter is extended at the 5′ end by 51 or fewer nucleotides occurring in a sequence encoding a non-structural protein (e.g., an alphavirus NSP4). In one example, the extended SG promoter is a minimal SG promoter extended at the 5′ end by no more than 51 nucleotides occurring in a sequence encoding a non-structural protein (e.g., an alphavirus NSP4). In one example, the extended SG promoter is encoded by a sequence comprising or consisting of a sequence set forth in SEQ ID NO: 14 extended at the 5′ end by no more than 51 nucleotides occurring in a sequence encoding a non-structural protein (e.g., an alphavirus NSP4). For example, the extended SG promoter is no more than 100 nucleotides in length. In one example, the extended SG promoter is encoded by a sequence comprising or consisting of nucleotides 2 to 101 of SEQ ID NO: 18.

In one example, the SG promoter is extended at the 5′ end by about 5 nucleotides to about 20 nucleotides, for example by about 5 nucleotides, or about 10 nucleotides, or about 12, or about 15 nucleotides, or about 20 nucleotides, occurring in a sequence encoding a non-structural protein (e.g., an alphavirus NSP4). In another example, the SG promoter is extended at the 5′ end by about 20 to about 35 nucleotides, for example, by about 25 nucleotides or about 27 nucleotides, or about 30 nucleotides, or about 35 nucleotides, occurring in a sequence encoding a non-structural protein (e.g., an alphavirus NSP4).

In one example, the SG promoter is extended at the 5′ end by about 12 nucleotides occurring in a sequence encoding a non-structural protein (e.g., an alphavirus NSP4). In one example, the extended SG promoter is encoded by a sequence set forth in SEQ ID NO: 14 extended at the 5′ end by 12 nucleotides occurring in a sequence encoding a non-structural protein (e.g., an alphavirus NSP4). For example, the extended SG promoter is no more than 61 nucleotides in length. In one example, the extended SG promoter is encoded by a sequence comprising or consisting of nucleotides 41 to 101 of SEQ ID NO: 18. In another example, the extended SG promoter is encoded by a sequence comprising or consisting of a sequence set forth in SEQ ID NO: 15.

In one example, the SG promoter is extended at the 5′ end by about 31 nucleotides occurring in a sequence encoding a non-structural protein (e.g., an alphavirus NSP4). In one example, the extended SG promoter is encoded by a sequence set forth in SEQ ID NO: 14 extended at the 5′ end by 31 nucleotides occurring in a sequence encoding a non-structural protein (e.g., an alphavirus NSP4). For example, the extended SG promoter is no more than 80 nucleotides in length. In one example, the extended SG promoter is encoded by a sequence comprising or consisting of nucleotides 22 to 101 of SEQ ID NO: 18. In another example, the extended SG promoter is encoded by a sequence comprising or consisting of a sequence set forth in SEQ ID NO: 16.

In one example, the extended SG promoter comprises a repeat sequence corresponding to nucleotides 66 to 75 of SEQ ID NO: 18. For example, the extended SG promoter is encoded by a sequence comprising nucleotides 50 to 75 of SEQ ID NO: 18 and nucleotides 66 to 101 of SEQ ID NO: 18. For example, the extended SG promoter is encoded by a sequence set forth in SEQ ID NO: 28.

In one example, the IRES is an IRES from encephalomyocarditis virus (EMCV), poliovirus (PV), human enterovirus, foot-and-mouth disease virus (FMDV), hepatitis C virus (HCV), classical swine fever virus (CSFV), murine leukemia virus (MLV), simian immunodeficiency virus (SIV), Eukaryotic translation initiation factor 4G (elF4G), Death-associated protein 5 (DAP5), cellular Myc (c-Myc), NF-κB-repressing factor (NRF), vascular endothelial growth factor (VEGF), fibroblast growth factor (FGF-2), platelet-derived growth factor B (PDGF B), Antennapedia, X-linked inhibitor of apoptosis (XIAP or Apaf-1), immunoglobulin heavy-chain binding protein BiP, or fibroblast growth factor 1a (FGF1A), GTX, or a combination thereof.

In one example, the IRES is a wild-type IRES derived from encephalomyocarditis virus (EMCV). For example, the wild-type EMCV IRES comprises a sequence set forth in SEQ ID NO: 17.

In one example, the first and/or second nucleotide sequence and/or the one or more additional nucleotide sequences are codon optimized.

In one example, the G/C content of the first and/or second nucleotide sequence and/or the one or more additional nucleotide sequences are modified.

In one example, the G/C content of the first and/or second nucleotide sequence and/or the one or more additional nucleotide sequences are increased by at least 5% compared to the G/C content of the unmodified sequence. For example, the G/C content of the first and/or second nucleotide sequence and/or the one or more additional nucleotide sequences are increased by at least 10%, or 15%, or 20%, or 25%, or 30%, or 35%, or 40% compared to the G/C content of the unmodified sequence.

In one example, the polynucleotide comprises at least one chemically modified nucleotide.

In one example, the chemically modified nucleotide is selected from the group consisting of N6,2′-O-dimethyl-adenosine (m6Am), 5-methyluridine (m5U), N4-acetylcytidine (ac4C), 2-thiocytidine (s2C), 2-thiouridine (s2U), 5-methylcytidine (m5C), N6-methyladenosine (m6a), pseudouridine (W), 1-methylpseudouridine (mly), and combinations thereof. For example, the chemically modified nucleotide is N6,2′-O-dimethyl-adenosine (m6Am). For example, the chemically modified nucleotide is 5-methyluridine (m5U). For example, the chemically modified nucleotide is N4-acetylcytidine (ac4C). For example, the chemically modified nucleotide is 2-thiocytidine (s2C). For example, the chemically modified nucleotide is 2-thiouridine (s2U). For example, the chemically modified nucleotide is 5-methylcytidine (m5C). For example, the chemically modified nucleotide is N6-methyladenosine (m6a). For example, the chemically modified nucleotide is pseudouridine (ψ). For example, the chemically modified nucleotide is 1-methylpseudouridine (m1ψ).

In one example, the first nucleotide sequence comprises the 5′-UTR of haptoglobin (HP), fibrinogen beta chain (FGB), haptoglobin-related protein (HPR), albumin (ALB), complement component 3 (C3), fibrinogen alpha chain (FGA), alpha 6 collagen (Col6A), alpha-1-antitrypsin (SERPINA1), alpha-1-antichymotrypsin (SERPINA3) a fragment and/or a variant thereof.

In one example, the 5′UTR is a 5′UTR of a Venezuelan equine encephalitis virus (VEEV) or modified forms thereof. For example, the 5′UTR comprises a sequence set forth in SEQ ID NO: 26.

In one example, the 5′-UTR, the fragment and/or the variant thereof is between 40 and 2000 nucleotides in length. For example, the 5′-UTR, the fragment and/or the variant thereof is between 40 and 100 nucleotides in length. For example, the 5′-UTR, the fragment and/or the variant thereof is between 100 and 250 nucleotides in length. For example, the 5′-UTR, the fragment and/or the variant thereof is between 250 and 500 nucleotides in length. For example, the 5′-UTR, the fragment and/or the variant thereof is between 500 and 750 nucleotides in length. For example, the 5′-UTR, the fragment and/or the variant thereof is between 750 and 1000 nucleotides in length. For example, the 5′-UTR, the fragment and/or the variant thereof is between 1000 and 1250 nucleotides in length. For example, the 5′-UTR, the fragment and/or the variant thereof is between 1250 and 1500 nucleotides in length. For example, the 5′-UTR, the fragment and/or the variant thereof is between 1500 and 1750 nucleotides in length. For example, the 5′-UTR, the fragment and/or the variant thereof is between 1750 and 2000 nucleotides in length.

In one example, the 5′-UTR, the fragment and/or the variant thereof comprises a nucleotide sequence at least 90% identical to a nucleotide sequence set forth in any one of SEQ ID NO: 22 to 26. For example, the 5′-UTR, the fragment and/or the variant thereof comprises a nucleotide sequence at least 90%, or 91%, or 92%, or 93%, or 94%, or 95%, or 96%, or 97%, or 98%, or 99% identical to a nucleotide sequence set forth in any one of SEQ ID NO: 22 to 26.

In one example, the polynucleotide comprises a combination of two or more 5′-UTRs, fragments and/or variants thereof. In one example, the two or more 5′-UTRs are the same. In one example, the two or more 5′-UTRs are different.

In one example, the nucleotide sequence comprising the 5′UTR comprises at least one microRNA binding site, an AU rich element (ARE), a GC-rich element, a stem loop, and combinations thereof. In one example, the nucleotide sequence comprises a microRNA binding site. In one example, the nucleotide sequence comprises an AU rich element (ARE). In one example, the nucleotide comprises a GC-rich element. In one example, the nucleotide sequence comprises a stem loop. For example, the stem loop is a histone stem loop.

In one example, the polynucleotide further comprises a nucleotide sequence comprising a 3′UTR. In one example, the nucleotide sequence comprising the 3′UTR is located 3′ of the second or the one or more additional nucleotide sequences. For example, the nucleotide sequence comprising the 3′UTR is located 3′ of the second nucleotide sequence. In one example, the 3′UTR comprises a 3′-UTR of arachidonate 5-lipoxygenase (ALOX5), alpha I collagen (COL1A1), tyrosine hydroxylase (TH) gene, amino-terminal enhancer of split (AES), human mitochondrial 12S rRNA (mtRNR1), a fragment and/or a variant thereof.

In one example, the 3′UTR is a 3′UTR of a Sindbis virus (SINV) or modified forms thereof. For example, the 3′UTR comprises a sequence set forth in SEQ ID NO: 27.

In one example, the 3′UTR, the fragment and/or the variant thereof is between 40 and 400 nucleotides in length. For example, the 3′-UTR is between 40 and 50, or 50 and 60, or 60 and 70, or 70 and 80, or 80 and 90, or 90 and 100, or 100 and 125, or 125 and 150, or 150 and 175, or 175 and 200, or 200 and 225, or 225 and 250, or 250 and 275, or 275 and 300, or 300 and 325, or 325 and 350, or 350 and 375, or 375 and 400 nucleotides in length. For example, the 3′-UTR, the fragment and/or the variant thereof is between 40 and 50 nculeotides in length. For example, the 3′-UTR, the fragment and/or the variant thereof is between 50 and 60 nucleotides in length. For example, the 3′-UTR, the fragment and/or the variant thereof is between 60 and 70 nucleotides in length. For example, the 3′-UTR, the fragment and/or the variant thereof is between 70 and 80 nucleotides in length. For example, the 3′-UTR, the fragment and/or the variant thereof is between 80 and 90 nucleotides in length. For example, the 3′-UTR, the fragment and/or the variant thereof is between 90 and 100 nucleotides in length. For example, the 3′-UTR, the fragment and/or the variant thereof is between 100 and 125 nucleotides in length. For example, the 3′-UTR, the fragment and/or the variant thereof is between 125 and 150 nucleotides in length. For example, the 3′-UTR, the fragment and/or the variant thereof is between 150 and 175 nucleotides in length. For example, the 3′-UTR, the fragment and/or the variant thereof is between 175 and 200 nucleotides in length. For example, the 3′-UTR, the fragment and/or the variant thereof is between 200 and 225 nucleotides in length. For example, the 3′-UTR, the fragment and/or the variant thereof is between 225 and 250 nucleotides in length. For example, the 3′-UTR, the fragment and/or the variant thereof is between 250 and 275 nucleotides in length. For example, the 3′-UTR, the fragment and/or the variant thereof is between 275 and 300 nucleotides in length. For example, the 3′-UTR, the fragment and/or the variant thereof is between 300 and 325 nucleotides in length. For example, the 3′-UTR, the fragment and/or the variant thereof is between 325 and 350 nucleotides in length. For example, the 3′-UTR, the fragment and/or the variant thereof is between 350 and 375 nucleotides in length. For example, the 3′-UTR, the fragment and/or the variant thereof is between 375 and 400 nucleotides in length.

In one example, the polynucleotide comprises a combination of two or more 3′-UTRs, fragments and/or variants thereof. In one example, the two or more 3′-UTRs are the same. In one example, the two or more 3′-UTRs are different.

In one example, the nucleotide sequence comprising the 3′UTR, the fragment and/or variant thereof comprises at least one microRNA binding site, an AU rich element (ARE), a GC-rich element, a triple helix, a stem loop, one or more stop codons and combinations thereof. In one example, the nucleotide sequence comprises a microRNA binding site. In one example, the nucleotide sequence comprises an AU rich element (ARE). In one example, the nucleotide sequence comprises a GC-rich element. In one example, the nucleotide sequence comprises a triple helix. In one example, the nucleotide sequence comprises a stem loop. For example, the stem loop is a histone stem loop. In one example, the nucleotide sequence comprises one or more stop codons. For example, the one or more stop codons are located at the 5′ end of the 3′-UTR.

In one example, the polynucleotide comprises a nucleotide sequence comprising one or more 3′ tailing sequences located at the 3′ end of the nucleotide sequence comprising the 3′UTR. In one example, the one or more 3′ tailing sequences are selected from the group consisting of a poly-A sequence, polyadenylation signal, a G-quadruplex, a poly-C sequence, a stem loop and combinations thereof. For example, the 3′ tailing sequence comprises a poly-A sequence. In one example, the 3′ tailing sequence comprises a polyadenylation signal. In one example, the 3′ tailing sequence comprises a G-quadruplex. In one example, the 3′ tailing sequence comprises a poly-C sequence. In one example, the 3′ tailing sequence comprises a stem loop. For example, the stem loop is a histone stem loop. In one example, the 3′ tailing sequence comprises a poly-A sequence and a G-quadruplex. In one example, the 3′ tailing sequence comprises a stem loop (e.g., a histone stem loop) and a poly-A sequence.

In one example, the one or more 3′ tailing sequences comprises one or more poly-A sequences each comprising between 10 and 300 consecutive adenosine nucleotides. For example, the poly-A sequences each comprises between 10 and 20, or 20 and 30, or 30 and 40, or 40 and 50, or 50 and 60, or 60 and 70, or 70 and 80, or 80 and 90, or 90 and 100, or 100 and 125, or 125 and 150, or 150 and 175, or 175 and 200, or 200 and 225, or 225 and 250, or 250 and 275, or 275 and 300 consecutive adenosine nucleotides. For example, the one or more poly-A sequences each comprises between 10 and 20 consecutive adenosine nucleotides. For example, the one or more poly-A sequences each comprises between 20 and 30 consecutive adenosine nucleotides. For example, the one or more poly-A sequences each comprises between 30 and 40 consecutive adenosine nucleotides. For example, the one or more poly-A sequences each comprise 36 consecutive adenosine nucleotides. For example, the one or more poly-A sequences each comprises between 40 and 50 consecutive adenosine nucleotides. For example, the one or more poly-A sequences each comprises between 50 and 60 consecutive adenosine nucleotides. For example, the one or more poly-A sequences each comprises between 60 and 70 consecutive adenosine nucleotides. For example, the one or more poly-A sequences each comprises between 70 and 80 consecutive adenosine nucleotides. For example, the one or more poly-A sequences each comprises between 80 and 90 consecutive adenosine nucleotides. For example, the one or more poly-A sequences each comprises between 90 and 100 consecutive adenosine nucleotides. For example, the one or more poly-A sequences each comprises between 100 and 125 consecutive adenosine nucleotides. For example, the one or more poly-A sequences each comprises between 125 and 150 consecutive adenosine nucleotides. For example, the one or more poly-A sequences each comprises between 150 and 175 consecutive adenosine nucleotides. For example, the one or more poly-A sequences each comprises between 175 and 200 consecutive adenosine nucleotides. For example, the one or more poly-A sequences each comprises between 200 and 225 consecutive adenosine nucleotides. For example, the one or more poly-A sequences each comprises between 225 and 250 consecutive adenosine nucleotides. For example, the one or more poly-A sequences each comprises between 250 and 275 consecutive adenosine nucleotides. For example, the one or more poly-A sequences each comprises between 275 and 300 consecutive adenosine nucleotides.

In one example, the one or more poly-A sequence each comprises 10, or 20, or 30, or 40, or 50, or 60, or 70, or 80, or 90, or 100, or 125, or 150, or 175, or 200, or 225, or 250, or 275, or 300 consecutive adenosine nucleotides. For example, the one or more poly-A sequence each comprises 10 consecutive adenosine nucleotides. For example, the one or more poly-A sequence each comprises 20 consecutive adenosine nucleotides. For example, the one or more poly-A sequence each comprises 30 consecutive adenosine nucleotides. For example, the one or more poly-A sequence each comprises 40 consecutive adenosine nucleotides. For example, the one or more poly-A sequence each comprises 50 consecutive adenosine nucleotides. For example, the one or more poly-A sequence each comprises 60 consecutive adenosine nucleotides. For example, the one or more poly-A sequence each comprises 70 consecutive adenosine nucleotides. For example, the one or more poly-A sequence each comprises 80 consecutive adenosine nucleotides. For example, the one or more poly-A sequence each comprises 90 consecutive adenosine nucleotides. For example, the one or more poly-A sequence each comprises 100 consecutive adenosine nucleotides. For example, the one or more poly-A sequence each comprises 125 consecutive adenosine nucleotides. For example, the one or more poly-A sequence each comprises 150 consecutive adenosine nucleotides. For example, the one or more poly-A sequence each comprises 175 consecutive adenosine nucleotides. For example, the one or more poly-A sequence each comprises 200 consecutive adenosine nucleotides. For example, the one or more poly-A sequence each comprises 225 consecutive adenosine nucleotides. For example, the one or more poly-A sequence each comprises 250 consecutive adenosine nucleotides. For example, the one or more poly-A sequence each comprises 275 consecutive adenosine nucleotides. For example, the one or more poly-A sequence each comprises 300 consecutive adenosine nucleotides.

In one example, the poly-A sequence comprises 36 consecutive adenosine nucleotides. For example, the poly-A sequence comprises a sequence set forth in SEQ ID NO: 29.

In one example, the one or more poly-A sequences is separated by an interrupting linker. For example, the 3′ tailing sequence comprises, in order of 5′ to 3′: a poly-A sequence comprising consecutive adenosine nucleotides, an interrupting linker, and a further poly-A sequence comprising consecutive adenosine nucleotides.

In one example, the interrupting linker is from 10 to 50, or 50 to 100, or 100 to 150 nucleotides in length. For example, the interrupting linker is from 10 to 50 nucleotides in length. For example, the interrupting linker is from 50 to 100 nucleotides in length. For example, the interrupting linker is from 100 to 150 nucleotides in length.

In one example, the interrupting linker is 1, or 2, or 3, or 4, or 5, or 6, or 7, or 8, or 9, or 10, or 11, or 12, or 13, or 14, or 15, or 16, or 17, or 18, or 19, or 20, or 25, or 30, or 35, or 40, or 45, or 50, or 55, or 60, or 65, or 70, or 75, or 80, or 85, or 90, or 95, or 100, or 110, or 120, or 130, or 140, or 150 nucleotides in length. For example, the interrupting linker is 1 nucleotide in length. For example, the interrupting linker is 2 nucleotides in length. For example, the interrupting linker is 3 nucleotides in length. For example, the interrupting linker is 4 nucleotides in length. For example, the interrupting linker is 5 nucleotides in length. For example, the interrupting linker is 6 nucleotides in length. For example, the interrupting linker is 7 nucleotides in length. For example, the interrupting linker is 8 nucleotides in length. For example, the interrupting linker is 9 nucleotides in length. For example, the interrupting linker is 10 nucleotides in length. For example, the interrupting linker is 11 nucleotides in length. For example, the interrupting linker is 12 nucleotides in length. For example, the interrupting linker is 13 nucleotides in length. For example, the interrupting linker is 14 nucleotides in length. For example, the interrupting linker is 15 nucleotides in length. For example, the interrupting linker is 16 nucleotides in length. For example, the interrupting linker is 17 nucleotides in length. For example, the interrupting linker is 18 nucleotides in length. For example, the interrupting linker is 19 nucleotides in length. For example, the interrupting linker is 20 nucleotides in length. For example, the interrupting linker is 25 nucleotides in length. For example, the interrupting linker is 30 nucleotides in length. For example, the interrupting linker is 35 nucleotides in length. For example, the interrupting linker is 40 nucleotides in length. For example, the interrupting linker is 45 nucleotides in length. For example, the interrupting linker is 50 nucleotides in length. For example, the interrupting linker is 55 nucleotides in length. For example, the interrupting linker is 60 nucleotides in length. For example, the interrupting linker is 65 nucleotides in length. For example, the interrupting linker is 70 nucleotides in length. For example, the interrupting linker is 75 nucleotides in length. For example, the interrupting linker is 80 nucleotides in length. For example, the interrupting linker is 85 nucleotides in length. For example, the interrupting linker is 90 nucleotides in length. For example, the interrupting linker is 95 nucleotides in length. For example, the interrupting linker is 100 nucleotides in length. For example, the interrupting linker is 110 nucleotides in length. For example, the interrupting linker is 120 nucleotides in length. For example, the interrupting linker is 130 nucleotides in length. For example, the interrupting linker is 140 nucleotides in length. For example, the interrupting linker is 150 nucleotides in length.

In one example, the interrupting linker is 10 nucleotides in length. In one example, the interrupting linker comprises or consists of the nucleotide sequence set forth in SEQ ID NO: 21. For example, the interrupting linker comprises or consists of a nucleotide sequence GCAUAUGACU.

In one example, the 3′ tailing sequence comprises, in order of 5′ to 3′: a poly-A sequence comprising 30 consecutive adenosine nucleotides, an interrupting linker of 10 nucleotides, and a further poly-A sequence comprising 70 consecutive adenosine nucleotides.

In one example, the 3′ tailing sequence comprises, in order of 5′ to 3′: a poly-A sequence comprising 30 consecutive adenosine nucleotides, an interrupting linker comprising or consisting of the nucleotide sequence set forth in SEQ ID NO: 21, and a further poly-A sequence comprising 70 consecutive adenosine nucleotides.

In one example, the polynucleotide comprises, in order from 5′ to 3′:
a) a 5′-UTR, fragment and/or variant thereof;
b) a regulatory element selected from the group consisting of a Kozak consensus sequence, an IRES, a SG promoter and combinations thereof;
c) a first nucleotide sequence encoding an antigen of interest;
d) a second nucleotide sequence encoding an adjuvant operably linked to a regulatory element selected from the group consisting of a SG promoter and an IRES, wherein the adjuvant is a cathelicidin or a fragment thereof;
e) a 3′-UTR, fragment and/or variant thereof; and
f) one or more 3′ tailing sequences selected from the group consisting of a poly-A sequence, polyadenylation signal, a G-quadruplex, a poly-C sequence, a stem loop and combinations thereof.

In one example, the polynucleotide comprises, in order from 5′ to 3′:
a) a 5′-UTR, fragment and/or variant thereof;
b) a regulatory element selected from the group consisting of a Kozak consensus sequence, an IRES, a SG promoter and combinations thereof;
c) a first nucleotide sequence encoding an adjuvant, wherein the adjuvant is a cathelicidin or a fragment thereof;
d) a second nucleotide sequence encoding an antigen of interest operably linked to a regulatory element selected from the group consisting of a SG promoter and an IRES;
e) a 3′-UTR, fragment and/or variant thereof; and
f) one or more 3′ tailing sequences selected from the group consisting of a poly-A sequence, polyadenylation signal, a G-quadruplex, a poly-C sequence, a stem loop and combinations thereof.

In one example, the RNA comprises, in order from 5′ to 3′:
a) a 5′-UTR, fragment and/or variant thereof;
b) a regulatory element selected from the group consisting of a Kozak consensus sequence, an IRES, a SG promoter and combinations thereof;
c) a first nucleotide sequence encoding an adjuvant, wherein the adjuvant is a cathelicidin or a fragment thereof;
d) a second nucleotide sequence encoding an antigen of interest operably linked to a regulatory element selected from the group consisting of a SG promoter and an IRES;
e) a 3′-UTR, fragment and/or variant thereof; and
f) one or more 3′ tailing sequences selected from the group consisting of a poly-A sequence, polyadenylation signal, a G-quadruplex, a poly-C sequence, a stem loop and combinations thereof.

In one example, the RNA comprises, in order from 5′ to 3′:
a) a 5′-UTR, fragment and/or variant thereof;
b) a regulatory element selected from the group consisting of a Kozak consensus sequence, an IRES, a SG promoter and combinations thereof;
c) a first nucleotide sequence encoding an antigen of interest;
d) a second nucleotide sequence encoding an adjuvant operably linked to a regulatory element selected from the group consisting of a SG promoter and an IRES, wherein the adjuvant is a cathelicidin or a fragment thereof;
e) a 3′-UTR, fragment and/or variant thereof; and
f) one or more 3′ tailing sequences selected from the group consisting of a poly-A sequence, polyadenylation signal, a G-quadruplex, a poly-C sequence, a stem loop and combinations thereof.

In one example, the cRNA comprises, in order from 5′ to 3′:
a) a 5′-UTR, fragment and/or variant thereof;
b) a regulatory element selected from the group consisting of a Kozak consensus sequence, an IRES, a SG promoter and combinations thereof;
c) a first nucleotide sequence encoding an antigen of interest;
d) a second nucleotide sequence encoding an adjuvant operably linked to a regulatory element selected from the group consisting of a SG promoter and an IRES, wherein the adjuvant is a cathelicidin or a fragment thereof;
e) a 3′-UTR, fragment and/or variant thereof; and
f) one or more 3′ tailing sequences selected from the group consisting of a poly-A sequence, polyadenylation signal, a G-quadruplex, a poly-C sequence, a stem loop and combinations thereof.

In one example, the cRNA comprises, in order from 5′ to 3′:
a) a 5′-UTR, fragment and/or variant thereof;
b) a regulatory element selected from the group consisting of a Kozak consensus sequence, an IRES, a SG promoter and combinations thereof;
c) a first nucleotide sequence encoding an adjuvant, wherein the adjuvant is a cathelicidin or a fragment thereof;
d) a second nucleotide sequence encoding an antigen of interest operably linked to a regulatory element selected from the group consisting of a SG promoter and an IRES;
e) a 3′-UTR, fragment and/or variant thereof; and
f) one or more 3′ tailing sequences selected from the group consisting of a poly-A sequence, polyadenylation signal, a G-quadruplex, a poly-C sequence, a stem loop and combinations thereof.

In one example, the self-replicating RNA comprises, in order from 5′ to 3′:
a) a 5′-UTR, fragment and/or variant thereof;
b) a regulatory element selected from the group consisting of a Kozak consensus sequence, an IRES, a SG promoter and combinations thereof;
c) the first nucleotide sequence encoding an antigen of interest;
d) the second nucleotide sequence encoding an adjuvant operably linked to a regulatory element selected from the group consisting of a SG promoter and an IRES, wherein the adjuvant is a cathelicidin or a fragment thereof;
e) a 3′-UTR, fragment and/or variant thereof; and
f) one or more 3′ tailing sequences selected from the group consisting of a poly-A sequence, polyadenylation signal, a G-quadruplex, a poly-C sequence, a stem loop and combinations thereof.

In one example, the self-replicating RNA comprises, in order from 5′ to 3′:
a) a 5′-UTR, fragment and/or variant thereof;
b) a regulatory element selected from the group consisting of a Kozak consensus sequence, an IRES, a SG promoter and combinations thereof;
c) a first nucleotide sequence encoding an adjuvant, wherein the adjuvant is a cathelicidin or a fragment thereof;
d) a second nucleotide sequence encoding an antigen of interest operably linked to a regulatory element selected from the group consisting of a SG promoter and an IRES;
e) a 3′-UTR, fragment and/or variant thereof; and
f) one or more 3′ tailing sequences selected from the group consisting of a poly-A sequence, polyadenylation signal, a G-quadruplex, a poly-C sequence, a stem loop and combinations thereof.

In one example, the self-replicating RNA of the present disclosure comprises, in order from 5′ to 3′:
a) a first nucleotide sequence encoding an antigen of interest operably linked to a minimal SG promoter; and a second nucleotide sequence encoding an adjuvant operably linked to a minimal SG promoter, wherein the adjuvant is a cathelicidin or a fragment thereof; or
b) a first nucleotide sequence encoding an antigen of interest operably linked to a minimal SG promoter; and a second nucleotide sequence encoding an adjuvant operably linked to an extended SG promoter, wherein the adjuvant is a cathelicidin or a fragment thereof; or
c) a first nucleotide sequence encoding an antigen of interest operably linked to a minimal SG promoter; and a second nucleotide sequence encoding an adjuvant operably linked to an a wild-type EMCV IRES, wherein the adjuvant is a cathelicidin or a fragment thereof.

In one example, the self-replicating RNA of the present disclosure comprises, in order from 5′ to 3′:
a) a first nucleotide sequence encoding an adjuvant operably linked to a minimal SG promoter; and a second nucleotide sequence encoding an antigen of interest operably linked to a minimal SG promoter, wherein the adjuvant is a cathelicidin or a fragment thereof; or
b) a first nucleotide sequence encoding an adjuvant operably linked to a minimal SG promoter; and a second nucleotide sequence encoding an antigen of interest operably linked to an extended SG promoter, wherein the adjuvant is a cathelicidin or a fragment thereof; or
c) a first nucleotide sequence encoding an adjuvant operably linked to a minimal SG promoter; and a second nucleotide sequence encoding an antigen of interest operably linked to an a wild-type EMCV IRES, wherein the adjuvant is a cathelicidin or a fragment thereof.

In one example, the self-replicating RNA of the present disclosure comprises, in order from 5′ to 3′: a first nucleotide sequence encoding an antigen of interest operably linked to a minimal SG promoter; and a second nucleotide sequence encoding an adjuvant operably linked to a minimal SG promoter, wherein the adjuvant is a cathelicidin or a fragment thereof.

In one example, the self-replicating RNA of the present disclosure comprises, in order from 5′ to 3′: a first nucleotide sequence encoding an adjuvant operably linked to a minimal SG promoter; and a second nucleotide sequence encoding an antigen of interest operably linked to a minimal SG promoter, wherein the adjuvant is a cathelicidin or a fragment thereof.

For example, the self-replicating RNA of the present disclosure comprises, in order from 5′ to 3′: a first nucleotide sequence encoding an antigen of interest operably linked to a minimal SG promoter comprising a sequence set forth in SEQ ID NO: 14; and a second nucleotide sequence encoding a cathelicidin operably linked to a minimal SG promoter comprising a sequence set forth in SEQ ID NO: 14.

For example, the self-replicating RNA of the present disclosure comprises, in order from 5′ to 3′: a first nucleotide sequence encoding a cathelicidin or a fragment thereof operably linked to a minimal SG promoter comprising a sequence set forth in SEQ ID NO: 14; and a second nucleotide sequence encoding an antigen of interest operably linked to a minimal SG promoter comprising a sequence set forth in SEQ ID NO: 14.

In one example, the self-replicating RNA of the present disclosure comprises, in order from 5′ to 3′: a first nucleotide sequence encoding an antigen of interest operably linked to a minimal SG promoter; and a second nucleotide sequence encoding an adjuvant operably linked to an extended SG promoter, wherein the adjuvant is a cathelicidin or a fragment thereof.

In one example, the self-replicating RNA of the present disclosure comprises, in order from 5′ to 3′: a first nucleotide sequence encoding an adjuvant operably linked to a minimal SG promoter; and a second nucleotide sequence encoding an antigen of interest operably linked to an extended SG promoter, wherein the adjuvant is a cathelicidin or a fragment thereof.

In one example, the self-replicating RNA of the present disclosure comprises, in order from 5′ to 3′: a first nucleotide sequence encoding an antigen of interest operably linked to a minimal SG promoter encoded by a sequence set forth in SEQ ID NO: 14; and a second nucleotide sequence encoding a cathelicidin or a fragment thereof operably linked to an extended SG promoter encoded by a sequence set forth in SEQ ID NO: 15.

In one example, the self-replicating RNA of the present disclosure comprises, in order from 5′ to 3′: a first nucleotide sequence encoding a cathelicidin operably linked to a minimal SG promoter encoded by a sequence set forth in SEQ ID NO: 14; and a second nucleotide sequence encoding an antigen of interest operably linked to an extended SG promoter encoded by a sequence set forth in SEQ ID NO: 15.

In one example, the self-replicating RNA of the present disclosure comprises, in order from 5′ to 3′: a first nucleotide sequence encoding an antigen of interest operably linked to a minimal SG promoter; and a second nucleotide sequence encoding an adjuvant operably linked to a wild-type EMCV IRES, wherein the adjuvant is a cathelicidin or a fragment thereof.

In one example, the self-replicating RNA of the present disclosure comprises, in order from 5′ to 3′: a first nucleotide sequence encoding an adjuvant operably linked to a minimal SG promoter; and a second nucleotide sequence encoding an antigen of interest operably linked to a wild-type EMCV IRES, wherein the adjuvant is a cathelicidin or a fragment thereof.

In one example, the self-replicating RNA of the present disclosure comprises, in order from 5′ to 3′: a first nucleotide sequence encoding antigen of interest operably linked to a minimal SG promoter encoded by a sequence set forth in SEQ ID NO: 14 and a second nucleotide sequence encoding a cathelicidin or a fragment thereof operably linked to a wild-type EMCV IRES encoded by a sequence set forth in SEQ ID NO: 17.

In one example, the self-replicating RNA of the present disclosure comprises, in order from 5′ to 3′: a first nucleotide sequence encoding a cathelicidin or a fragment thereof operably linked to a minimal SG promoter encoded by a sequence set forth in SEQ ID NO: 14 and a second nucleotide sequence encoding an antigen of interst operably linked to a wild-type EMCV IRES encoded by a sequence set forth in SEQ ID NO: 17.

In one example, the RNA further comprises a 5′ terminal cap structure.

In one example, the 5′ terminal cap structure is an endogenous cap or analogue thereof. For example, the 5′ terminal cap structure is an endogenous cap. For example, the 5′ terminal cap structure is an analogue of an endogenous cap.

In one example, the 5′ terminal cap structure comprise a guanine or guanine analogue thereof. For example, the 5′ terminal cap structure comprise a guanine. For example, the 5′ terminal cap structure comprise a guanine analogue of a guanine.

In one example, the 5′ terminal cap structure is selected from a group consisting of anti-reverse cap analogue (ARCA), N7,2′-O-dimethyl-guanosine (mCAP), inosine, N1-methyl-guanosine, 2′fluoro-guanosine, 7-deaza-guanosine, 8-oxo-guanosine, 2-amino-guanosine, LNA-guanosine, 2-azido-guanosine, N6,2′-O-dimethyladenosine, 7-methylguanosine (m7G), Cap1, and Cap2. For example, the 5′ terminal cap structure is anti-reverse cap analogue (ARCA). For example, the 5′ terminal cap structure is N7,2′-0-dimethyl-guanosine (mCAP). For example, the 5′ terminal cap structure is inosine. For example, the 5′ terminal cap structure is N1-methyl-guanosine. For example, the 5′ terminal cap structure is 2′fluoro-guanosine. For example, the 5′ terminal cap structure is 7-deaza-guanosine. For example, the 5′ terminal cap structure is 8-oxo-guanosine. For example, the 5′ terminal cap structure is 2-amino-guanosine. For example, the 5′ terminal cap structure is LNA-guanosine. For example, the 5′ terminal cap structure is 2-azido-guanosine. For example, the 5′ terminal cap structure is N6,2′-O-dimethyladenosine. For example, the 5′ terminal cap structure is 7-methylguanosine (m7G). For example, the 5′ terminal cap structure is Cap1. For example, the 5′ terminal cap structure is Cap2.

In one example, the 5′ terminal cap structure is linked to the 5′ end of the RNA by a 5′-5′-triphosphate linkage or a 5′-5′ phosphorothioate linkage. For example, the 5′ terminal cap structure is linked to the 5′ end of the RNA by a 5′-5′-triphosphate linkage. For example, the 5′ terminal cap structure is linked to the 5′ end of the RNA by a 5′-5′ phosphorothioate linkage.

In one example, the self-replicating RNA is from an alphavirus. For example, the alphavirus is selected from the group consisting of Semliki Forest virus (SFV), Sindbis virus (SINV), and Venezuelan equine encephalitis virus (VEEV) and combinations thereof.

In one example, the self-replicating RNA is from a Semliki Forest virus (SFV).

In one example, the self-replicating RNA is from a Sindbis virus (SINV).

In one example, the self-replicating RNA is from a Venezuelan equine encephalitis virus (VEEV).

In one example, the antigen is a viral antigen. For example, the viral antigen is from a respiratory virus. In one example, the respiratory virus is selected from the group consisting of influenza virus, respiratory syncytial virus, parainfluenza viruses, metapneumovirus, rhinovirus, coronaviruses, adenoviruses and bocaviruses.

In one example, the viral antigen is from an influenza virus.

In one example, the viral antigen is from a respiratory syncytial virus.

In one example, the viral antigen is from a parainfluenza virus.

In one example, the viral antigen is from a metapneumovirus.

In one example, the viral antigen is from a rhinovirus.

In one example, the viral antigen is from a coronavirus.

In one example, the viral antigen is from an adenovirus.

In one example, the viral antigen is from a bocavirus.

In one example, the antigen is a viral antigen from an influenza virus or a coronavirus.

In one example, the nucleotide sequence encoding a cathelicidin is selected from bovine cathelicidins: e.g., Bacl (Bactenecinl), Bac5, Bac7, indolicidin, BMAP-27 (bovine myeloid antimicrobial peptide 27) and BMAP-28; porcine cathelicidins: e.g., PR-39 (praline-arginine-rich 39 amino-acid peptide), PMAP-36 (porcine myeloid antimicrobial peptide 36), PMAP-37, PMAP-23, protegrins, and prophenins; rabbit cathelicidins: e.g., CAP18 (cationic antimicrobial protein 18); human cathelicidins: e.g., hCAP-18/FALL-39/LL-37 (human antimicrobial protein/C-terminal derived domains are called FALL-39 or LL-37); murine cathelicidins: e.g., mCRAMP (murine cathelin-related antimicrobial peptide), MCLP (murine cathelin-like protein); rat cathelicidins: e.g., rCRAMP (rat cathelin-related antimicrobial peptide) and; sheep cathelicidins: e.g., SMAP29 (sheep myeloid antimicrobial peptide 29) and SMAP34.

In one example, nucleotide sequence encoding a cathelicidin is selected from the group consisting of dododecapeptide, indolicidin, buCATHL4A, protegrin-1, PMAP-23, BMAP-27, eCATH-2, SMAP-29, mCRAMP, rCRAMP, PMAP-36, LL-37, CAP18-FV, PMAP-37, ttLL-37, eCATH-3, Bac7, prophenin-1 or a fragment thereof. In an example, the cathelicidin is a human cathelicidin or a fragment thereof. In another example, the cathelicidin is LL-37 or a fragment thereof.

In an example, the nucleotide sequence encoding a cathelicidin or a fragment thereof comprises a polynucleotide sequence having at least about 70%, at least about 75%, at least about 80%, at least about 85%, at least about 90% at least about 95%, at least about 96%, at least about 97%, at least about 98%, at least about 99% to any one of dododecapeptide, indolicidin, buCATHL4A, protegrin-1, PMAP-23, BMAP-27, eCATH-2, SMAP-29, mCRAMP, rCRAMP, PMAP-36, LL-37, CAP18-FV, PMAP-37, ttLL-37, eCATH-3, Bac7 or prophenin-1 or a fragment thereof.

In an example, the nucleotide sequence encoding a cathelicidin or a fragment thereof comprises a polynucleotide sequence having at least about 70%, at least about 75%, at least about 80%, at least about 85%, at least about 90% at least about 95%, at least about 96%, at least about 97%, at least about 98%, at least about 99% or 100% sequence identity to any one of SEQ ID NOs: 1, 4, 7 or 10.

In an example, the nucleotide sequence encoding a cathelicidin or a fragment thereof comprises a polynucleotide sequence having at least about 70%, at least about 75%, at least about 80%, at least about 85%, at least about 90% at least about 95%, at least about 96%, at least about 97%, at least about 98%, at least about 99% or 100% sequence identity to any one of SEQ ID NOs: 2, 5, 8 or 11.

In one example, the self-replicating RNA comprises or consists of a sequence according to SEQ ID NO: 31.

The present disclosure provides an immunogenic composition comprising a polynucleotide of the present disclosure. The present disclosure further provides an immunogenic composition comprising a RNA of the present disclosure. For example, the present disclosure provides an immunogenic composition comprising a cRNA of the present disclosure. The present disclosure also provides an immunogenic composition comprising a self-replicating RNA of the present disclosure. In an example, the composition of the present disclosure, when administered, is capable of inducing an immune response in the subject. For example, administration of the composition induces a humoral and/or a cell-mediated immune response. In one example, the composition induces a humoral immune response in the subject. In another example, the humoral immune response is an antibody-mediated immune response. In another example, the composition induces a cell-mediated immune response. For example, the cell-mediated immune response includes activation of antigen-specific cytotoxic T cells.

In one example, an immunogenic composition of the disclosure comprises multiple polynucleotides, wherein each polynucleotide encodes different polypeptide sequences. In another example, an immunogenic composition of the disclosure comprises multiple RNAs, wherein each RNA encodes different polypeptide sequences. In a further example, an immunogenic composition of the disclosure comprises multiple cRNAs, wherein each cRNA encodes different polypeptide sequences. In one example, an immunogenic composition comprises multiple multicistronic self-replicating RNAs, wherein each multicistronic self-replicating RNA encodes a polypeptide of an antigen of interest and a cathelicidin adjuvant described herein. In another example, an immunogenic composition of the disclosure comprises a plurality of self-replicating monocistronic RNAs, wherein each self-replicating RNA encodes different polypeptide sequences. For example, one self-replicating RNA encodes a polypeptide of an antigen of interest and a different self-replicating RNA encodes a cathelicidin adjuvant described herein.

The present disclosure also provides an immunogenic composition comprising a polynucleotide of the present disclosure and a protein adjuvant, wherein the protein adjuvant is a cathelicidin.

For example, the immunogenic composition comprises:
(i) a polynucleotide comprising a first nucleotide sequence encoding an antigen operably linked to a regulatory element; and
(ii) a cathelicidin polypeptide or a fragment thereof.

In another example, the immunogenic composition comprises:
(i) a RNA comprising a first nucleotide sequence encoding an antigen operably linked to a regulatory element; and
(ii) a cathelicidin polypeptide or a fragment thereof.

In another example, the immunogenic composition comprises:
(i) a cRNA comprising a first nucleotide sequence encoding an antigen operably linked to a regulatory element; and
(ii) a cathelicidin polypeptide or a fragment thereof.

In another example, the immunogenic composition comprises:
(i) a self-replicating RNA comprising a first nucleotide sequence encoding an antigen operably linked to a regulatory element; and
(ii) a cathelicidin polypeptide or a fragment thereof.

In an example, the regulatory element is selected from the group consisting of a promoter, optionally a subgenomic (SG) promoter, an internal ribosome entry site (IRES) and a Kozac consensus sequence or a combination thereof. In an example, the regulatory element is a SG promoter.

For example, the polynucleotide of the immunogenic composition comprises a first nucleotide sequence encoding an antigen operably linked to a regulatory element selected from a SG promoter, and IRES and/or a Kozac consensus sequence.

In another example, the RNA of the immunogenic composition comprises a first nucleotide sequence encoding an antigen operably linked to a regulatory element selected from a SG promoter, and IRES and/or a Kozac consensus sequence.

In another example, the cRNA of the immunogenic composition comprises a first nucleotide sequence encoding an antigen operably linked to a regulatory element selected from a SG promoter, and IRES and/or a Kozac consensus sequence.

In another example, the self-replicating RNA of the immunogenic composition comprises a first nucleotide sequence encoding an antigen operably linked to a regulatory element selected from a SG promoter, and IRES and/or a Kozac consensus sequence.

In one example, the immunogenic composition comprises:
(i) a polynucleotide comprising a first nucleotide sequence encoding an antigen operably linked to a regulatory element, and a second nucleotide sequence encoding an adjuvant operably linked to a regulatory element, wherein the adjuvant is a cathelicidin or a fragment thereof; and
(ii) a cathelicidin polypeptide or a fragment thereof.

In another example, the immunogenic composition comprises:
(i) a RNA comprising a first nucleotide sequence encoding an antigen operably linked to a regulatory element, and a second nucleotide sequence encoding an adjuvant operably linked to a regulatory element, wherein the adjuvant is a cathelicidin or a fragment thereof; and
(ii) a cathelicidin polypeptide or a fragment thereof.

In another example, the immunogenic composition comprises:
(i) a cRNA comprising a first nucleotide sequence encoding an antigen operably linked to a regulatory element, and a second nucleotide sequence encoding an adjuvant operably linked to a regulatory element, wherein the adjuvant is a cathelicidin or a fragment thereof; and
(ii) a cathelicidin polypeptide or a fragment thereof.

In another example, the immunogenic composition comprises:
(i) a self-replicating RNA comprising a first nucleotide sequence encoding an antigen operably linked to a regulatory element, and a second nucleotide sequence encoding an adjuvant operably linked to a regulatory element, wherein the adjuvant is a cathelicidin or a fragment thereof; and
(ii) a cathelicidin polypeptide or a fragment thereof.

Accordingly, the present disclosure provides an immunogenic composition comprising:
(i) a RNA comprising: a) a first nucleotide sequence encoding a first antigen of interest; and b) a second nucleotide sequence encoding an adjuvant operably linked to a regulatory element selected from the group consisting of a SG promoter and an IRES, wherein the adjuvant is a cathelicidin or a fragment thereof; and
(ii) a cathelicidin polypeptide or a fragment thereof.

In one example, the RNA comprises, in order from 5′ to 3′: a) a first nucleotide sequence encoding a first antigen of interest; and b) a second nucleotide sequence encoding an adjuvant operably linked to a regulatory element selected from the group consisting of a SG promoter and an IRES, wherein the adjuvant is a cathelicidin or a fragment thereof.

In one example, the RNA comprises, in order from 5′ to 3′: a) a first nucleotide sequence encoding an adjuvant; and b) a second nucleotide sequence encoding an antigen of interest operably linked to a regulatory element selected from the group consisting of a SG promoter and an IRES, wherein the adjuvant is a cathelicidin or a fragment thereof.

The present disclosure further provides an immunogenic composition comprising:
(i) a self-replicating RNA comprising: a) a first nucleotide sequence encoding a first antigen of interest; and b) a second nucleotide sequence encoding an adjuvant operably linked to a regulatory element selected from the group consisting of a SG promoter and an IRES, wherein the adjuvant is a cathelicidin or a fragment thereof; and
(ii) a cathelicidin polypeptide or a fragment thereof.

In one example, the self-replicating RNA comprises, in order from 5′ to 3′: a) a first nucleotide sequence encoding a first antigen of interest; and b) a second nucleotide sequence encoding an adjuvant operably linked to a regulatory element selected from the group consisting of a SG promoter and an IRES, wherein the adjuvant is a cathelicidin or a fragment thereof.

In one example, the self-replicating RNA comprises, in order from 5′ to 3′: a) a first nucleotide sequence encoding an adjuvant; and b) a second nucleotide sequence encoding an antigen of interest operably linked to a regulatory element selected from the group consisting of a SG promoter and an IRES, wherein the adjuvant is a cathelicidin or a fragment thereof.

The present disclosure further provides an immunogenic composition comprising
(i) a RNA comprising: a) a first nucleotide sequence encoding a first antigen of interest operably linked to a regulatory element selected from the group consisting of a Kozak consensus sequence, an IRES, a SG promoter and combinations thereof; and b) a second nucleotide sequence encoding an adjuvant operably linked to a regulatory element selected from the group consisting of a SG promoter and an IRES, wherein the adjuvant is a cathelicidin or a fragment thereof; and
(ii) a cathelicidin polypeptide or a fragment thereof.

In one example, the RNA comprises, in order from 5′ to 3′: a) a first nucleotide sequence encoding a first antigen of interest operably linked to a regulatory element selected from the group consisting of a Kozak consensus sequence, an IRES, a SG promoter and combinations thereof; and b) a second nucleotide sequence encoding an adjuvant operably linked to a regulatory element selected from the group consisting of a SG promoter and an IRES, wherein the adjuvant is a cathelicidin or a fragment thereof.

In one example, the RNA comprises, in order from 5′ to 3′: a) a first nucleotide sequence encoding an adjuvant operably linked to a regulatory element selected from the group consisting of a Kozak consensus sequence, an IRES, a SG promoter and combinations thereof; and b) a second nucleotide sequence encoding an antigen of interest operably linked to a regulatory element selected from the group consisting of a SG promoter and an IRES, wherein the adjuvant is a cathelicidin or a fragment thereof.

The present disclosure provides an immunogenic composition comprising:
(i) a self-replicating RNA comprising: a) a first nucleotide sequence encoding a first antigen of interest operably linked to a regulatory element selected from the group consisting of a Kozak consensus sequence, an IRES, a SG promoter and combinations thereof; and b) a second nucleotide sequence encoding an adjuvant operably linked to a regulatory element selected from the group consisting of a SG promoter and an IRES, wherein the adjuvant is a cathelicidin or a fragment thereof; and
(ii) a cathelicidin polypeptide or a fragment thereof.

In one example, the self-replicating RNA comprises, in order from 5′ to 3′: a) a first nucleotide sequence encoding a first antigen of interest operably linked to a regulatory element selected from the group consisting of a Kozak consensus sequence, an IRES, a SG promoter and combinations thereof; and b) a second nucleotide sequence encoding an adjuvant operably linked to a regulatory element selected from the group consisting of a SG promoter and an IRES, wherein the adjuvant is a cathelicidin or a fragment thereof.

In one example, the self-replicating RNA comprises, in order from 5′ to 3′: a) a first nucleotide sequence encoding an adjuvant operably linked to a regulatory element selected from the group consisting of a Kozak consensus sequence, an IRES, a SG promoter and combinations thereof; and b) a second nucleotide sequence encoding an antigen of interest operably linked to a regulatory element selected from the group consisting of a SG promoter and an IRES, wherein the adjuvant is a cathelicidin or a fragment thereof.

The present disclosure provides an immunogenic composition comprising:
(i) a RNA comprising: a) a first nucleotide sequence encoding a first antigen of interest operably linked to a Kozak consensus sequence; and b) a second nucleotide sequence encoding an adjuvant operably linked to a regulatory element selected from the group consisting of a SG promoter and an IRES, wherein the adjuvant is a cathelicidin or a fragment thereof; and
(ii) a cathelicidin polypeptide or a fragment thereof.

In one example, the RNA comprises, in order from 5′ to 3′: a) a first nucleotide sequence encoding a first antigen of interest operably linked to a Kozak consensus sequence; and b) a second nucleotide sequence encoding an adjuvant operably linked to a regulatory element selected from the group consisting of a SG promoter and an IRES, wherein the adjuvant is a cathelicidin or a fragment thereof.

In one example, the RNA comprises, in order from 5′ to 3′: a) a first nucleotide sequence encoding an adjuvant operably linked to a Kozak consensus sequence; and b) a second nucleotide sequence encoding an antigen of interest operably linked to a regulatory element selected from the group consisting of a SG promoter and an IRES, wherein the adjuvant is a cathelicidin or a fragment thereof.

The present disclosure provides an immunogenic composition comprising:
(i) a RNA comprising: a) a first nucleotide sequence encoding a first antigen of interest operably linked to a Kozak consensus sequence and a SG promoter; and b) a second nucleotide sequence encoding an adjuvant operably linked to a regulatory element selected from the group consisting of a SG promoter and an IRES, wherein the adjuvant is a cathelicidin or a fragment thereof; and
(ii) a cathelicidin polypeptide or a fragment thereof.

In one example, the RNA comprises, in order from 5′ to 3′: a) a first nucleotide sequence encoding a first antigen of interest operably linked to a Kozak consensus sequence and a SG promoter; and b) a second nucleotide sequence encoding an adjuvant operably linked to a regulatory element selected from the group consisting of a SG promoter and an IRES, wherein the adjuvant is a cathelicidin or a fragment thereof.

In one example, the RNA comprises, in order from 5′ to 3′: a) a first nucleotide sequence encoding an adjuvant operably linked to a Kozak consensus sequence and a SG promoter; and b) a second nucleotide sequence encoding an antigen of interest operably linked to a regulatory element selected from the group consisting of a SG promoter and an IRES, wherein the adjuvant is a cathelicidin or a fragment thereof.

The present disclosure provides an immunogenic composition comprising:
(i) a RNA comprising: a) a first nucleotide sequence encoding a first antigen of interest operably linked to a Kozak consensus sequence and an IRES; and b) a second nucleotide sequence encoding an adjuvant operably linked to a regulatory element selected from the group consisting of a SG promoter and an IRES, wherein the adjuvant is a cathelicidin or a fragment thereof; and
(ii) a cathelicidin polypeptide or a fragment thereof.

In one example, the RNA comprises, in order from 5′ to 3′: a) a first nucleotide sequence encoding a first antigen of interest operably linked to a Kozak consensus sequence and an IRES; and b) a second nucleotide sequence encoding an adjuvant operably linked to a regulatory element selected from the group consisting of a SG promoter and an IRES, wherein the adjuvant is a cathelicidin or a fragment thereof.

In one example, the RNA comprises, in order from 5′ to 3′: a) a first nucleotide sequence encoding an adjuvant operably linked to a Kozak consensus sequence and an IRES; and b) a second nucleotide sequence encoding an antigen of interest operably linked to a regulatory element selected from the group consisting of a SG promoter and an IRES, wherein the adjuvant is a cathelicidin or a fragment thereof.

The present disclosure provides an immunogenic composition comprising:
(i) a RNA comprising: a) a first nucleotide sequence encoding a first antigen of interest operably linked to a SG promoter; and b) a second nucleotide sequence encoding an adjuvant operably linked to a regulatory element selected from the group consisting of a SG promoter and an IRES, wherein the adjuvant is a cathelicidin or a fragment thereof; and
(ii) a cathelicidin polypeptide or a fragment thereof.

In one example, the RNA comprises, in order from 5′ to 3′: a) a first nucleotide sequence encoding a first antigen of interest operably linked to a SG promoter; and b) a second nucleotide sequence encoding an adjuvant operably linked to a regulatory element selected from the group consisting of a SG promoter and an IRES, wherein the adjuvant is a cathelicidin or a fragment thereof.

In one example, the RNA comprises, in order from 5′ to 3′: a) a first nucleotide sequence encoding an adjuvant operably linked to a SG promoter; and b) a second nucleotide sequence encoding an antigen of interest operably linked to a regulatory element selected from the group consisting of a SG promoter and an IRES, wherein the adjuvant is a cathelicidin or a fragment thereof.

The present disclosure provides an immunogenic composition comprising:
(i) a RNA comprising: a) a first nucleotide sequence encoding a first antigen of interest operably linked to an IRES; and b) a second nucleotide sequence encoding an adjuvant operably linked to a regulatory element selected from the group consisting of a SG promoter and an IRES, wherein the adjuvant is a cathelicidin or a fragment thereof;
(ii) a cathelicidin polypeptide or a fragment thereof.

In one example, the RNA comprises, in order from 5′ to 3′: a) a first nucleotide sequence encoding a first antigen of interest operably linked to an IRES; and b) a second nucleotide sequence encoding an adjuvant operably linked to a regulatory element selected from the group consisting of a SG promoter and an IRES, wherein the adjuvant is a cathelicidin or a fragment thereof.

In one example, the RNA comprises, in order from 5′ to 3′: a) a first nucleotide sequence encoding an adjuvant operably linked to an IRES; and b) a second nucleotide sequence encoding an antigen of interest operably linked to a regulatory element selected from the group consisting of a SG promoter and an IRES, wherein the adjuvant is a cathelicidin or a fragment thereof.

The present disclosure provides an immunogenic composition comprising:
(i) a self-replicating RNA comprising: a) a first nucleotide sequence encoding a first antigen of interest operably linked to a Kozak consensus sequence; and b) a second nucleotide sequence encoding an adjuvant operably linked to a regulatory element selected from the group consisting of a SG promoter and an IRES, wherein the adjuvant is a cathelicidin or a fragment thereof; and
(ii) a cathelicidin polypeptide or a fragment thereof.

In one example, the self-replicating RNA comprises, in order from 5′ to 3′: a) a first nucleotide sequence encoding a first antigen of interest operably linked to a Kozak consensus sequence; and b) a second nucleotide sequence encoding an adjuvant operably linked to a regulatory element selected from the group consisting of a SG promoter and an IRES, wherein the adjuvant is a cathelicidin or a fragment thereof.

In one example, the self-replicating RNA comprises, in order from 5′ to 3′: a) a first nucleotide sequence encoding an adjuvant operably linked to a Kozak consensus sequence; and b) a second nucleotide sequence encoding an antigen of interest operably linked to a regulatory element selected from the group consisting of a SG promoter and an IRES, wherein the adjuvant is a cathelicidin or a fragment thereof.

The present disclosure provides an immunogenic composition comprising:
(i) a self-replicating RNA comprising: a) a first nucleotide sequence encoding a first antigen of interest operably linked to a Kozak consensus sequence and a SG promoter; and b) a second nucleotide sequence encoding an adjuvant operably linked to a regulatory element selected from the group consisting of a SG promoter and an IRES, wherein the adjuvant is a cathelicidin or a fragment thereof; and
(ii) a cathelicidin polypeptide or a fragment thereof.

In one example, the self-replicating RNA comprises, in order from 5′ to 3′: a) a first nucleotide sequence encoding a first antigen of interest operably linked to a Kozak consensus sequence and a SG promoter; and b) a second nucleotide sequence encoding an adjuvant operably linked to a regulatory element selected from the group consisting of a SG promoter and an IRES, wherein the adjuvant is a cathelicidin or a fragment thereof.

In one example, the self-replicating RNA comprises, in order from 5′ to 3′: a) a first nucleotide sequence encoding an adjuvant operably linked to a Kozak consensus sequence and a SG promoter; and b) a second nucleotide sequence encoding an antigen of interest operably linked to a regulatory element selected from the group consisting of a SG promoter and an IRES, wherein the adjuvant is a cathelicidin or a fragment thereof.

The present disclosure provides an immunogenic composition comprising:
(i) a self-replicating RNA comprising: a) a first nucleotide sequence encoding a first antigen of interest operably linked to a Kozak consensus sequence and an IRES; and b) a second nucleotide sequence encoding an adjuvant operably linked to a regulatory element selected from the group consisting of a SG promoter and an IRES, wherein the adjuvant is a cathelicidin or a fragment thereof; and
(ii) a cathelicidin polypeptide or a fragment thereof.

In one example, the self-replicating RNA comprises, in order from 5′ to 3′: a) a first nucleotide sequence encoding a first antigen of interest operably linked to a Kozak consensus sequence and an IRES; and b) a second nucleotide sequence encoding an adjuvant operably linked to a regulatory element selected from the group consisting of a SG promoter and an IRES, wherein the adjuvant is a cathelicidin or a fragment thereof.

In one example, the self-replicating RNA comprises, in order from 5′ to 3′: a) a first nucleotide sequence encoding an adjuvant operably linked to a Kozak consensus sequence and an IRES; and b) a second nucleotide sequence encoding an antigen of interest operably linked to a regulatory element selected from the group consisting of a SG promoter and an IRES, wherein the adjuvant is a cathelicidin or a fragment thereof.

The present disclosure provides an immunogenic composition comprising:
(i) a self-replicating RNA comprising: a) a first nucleotide sequence encoding a first antigen of interest operably linked to a SG promoter; and b) a second nucleotide sequence encoding an adjuvant operably linked to a regulatory element selected from the group consisting of a SG promoter and an IRES, wherein the adjuvant is a cathelicidin or a fragment thereof; and
(ii) a cathelicidin polypeptide or a fragment thereof.

In one example, the self-replicating RNA comprises, in order from 5′ to 3′: a) a first nucleotide sequence encoding a first antigen of interest operably linked to a SG promoter; and b) a second nucleotide sequence encoding an adjuvant operably linked to a regulatory element selected from the group consisting of a SG promoter and an IRES, wherein the adjuvant is a cathelicidin or a fragment thereof.

In one example, the self-replicating RNA comprises, in order from 5′ to 3′: a) a first nucleotide sequence encoding an adjuvant operably linked to a SG promoter; and b) a second nucleotide sequence encoding an antigen of interest operably linked to a regulatory element selected from the group consisting of a SG promoter and an IRES, wherein the adjuvant is a cathelicidin or a fragment thereof.

The present disclosure provides an immunogenic composition comprising:
(i) a self-replicating RNA comprising: a) a first nucleotide sequence encoding a first antigen of interest operably linked to an IRES; and b) a second nucleotide sequence encoding an adjuvant operably linked to a regulatory element selected from the group consisting of a SG promoter and an IRES, wherein the adjuvant is a cathelicidin or a fragment thereof;
(ii) a cathelicidin polypeptide or a fragment thereof.

In one example, the self-replicating RNA comprises, in order from 5′ to 3′: a) a first nucleotide sequence encoding a first antigen of interest operably linked to an IRES; and b) a second nucleotide sequence encoding an adjuvant operably linked to a regulatory element selected from the group consisting of a SG promoter and an IRES, wherein the adjuvant is a cathelicidin or a fragment thereof.

In one example, the self-replicating RNA comprises, in order from 5′ to 3′: a) a first nucleotide sequence encoding an adjuvant operably linked to an IRES; and b) a second nucleotide sequence encoding an antigen of interest operably linked to a regulatory element selected from the group consisting of a SG promoter and an IRES, wherein the adjuvant is a cathelicidin or a fragment thereof.

In one example, the cathelicidin polypeptide or a fragment thereof is selected from bovine cathelicidins: e.g., Bacl (Bactenecinl), Bac5, Bac7, indolicidin, BMAP-27 (bovine myeloid antimicrobial peptide 27) and BMAP-28; porcine cathelicidins: e.g., PR-39 (praline-arginine-rich 39 amino-acid peptide), PMAP-36 (porcine myeloid antimicrobial peptide 36), PMAP-37, PMAP-23, protegrins, and prophenins; rabbit cathelicidins: e.g., CAP18 (cationic antimicrobial protein 18); human cathelicidins: e.g., hCAP-18/FALL-39/LL-37 (human antimicrobial protein/C-terminal derived domains are called FALL-39 or LL-37); murine cathelicidins: e.g., mCRAMP (murine cathelin-related antimicrobial peptide), MCLP (murine cathelin-like protein); rat cathelicidins: e.g., rCRAMP (rat cathelin-related antimicrobial peptide) and; sheep cathelicidins: e.g., SMAP29 (sheep myeloid antimicrobial peptide 29) and SMAP34.

In one example, the cathelicidin polypeptide is selected from the group consisting of dododecapeptide, indolicidin, buCATHL4A, protegrin-1, PMAP-23, BMAP-27, eCATH-2, SMAP-29, mCRAMP, rCRAMP, PMAP-36, LL-37, CAP18-FV, PMAP-37, ttLL-37, eCATH-3, Bac7, prophenin-1 or a fragment thereof. In an example, the cathelicidin polypeptide or a fragment thereof is a human cathelicidin or a fragment thereof. In another example, the cathelicidin polypeptide or a fragment thereof is LL-37 or a fragment thereof.

In an example, the cathelicidin polypeptide comprises an amino acid sequence having at least about 70%, at least about 75%, at least about 80%, at least about 85%, at least about 90% at least about 95%, at least about 96%, at least about 97%, at least about 98%, at least about 99% to any one of dododecapeptide, indolicidin, buCATHL4A, protegrin-1, PMAP-23, BMAP-27, eCATH-2, SMAP-29, mCRAMP, rCRAMP, PMAP-36, LL-37, CAP18-FV, PMAP-37, ttLL-37, eCATH-3, Bac7 or prophenin-1 or a fragment thereof.

In an example, the amino acid sequence of a cathelicidin polypeptide comprises a sequence having at least about 70%, at least about 75%, at least about 80%, at least about 85%, at least about 90% at least about 95%, at least about 96%, at least about 97%, at least about 98%, at least about 99% or 100% sequence identity to any one of SEQ ID NO: 3, 6, 9 or 12.

The present disclosure also provides a pharmaceutical composition comprising an immunogenic composition of the present disclosure and a pharmaceutically acceptable carrier. Pharmaceutically acceptable carriers suitable for use in the present disclosure will be apparent to the skilled person and/or are described herein.

In one example, the present disclosure provides a pharmaceutical composition comprising a polynucleotide comprising a first nucleotide sequence encoding an antigen operably linked to a regulatory element, and a second nucleotide sequence encoding a cathelicidin operably linked to a regulatory element, wherein the composition further comprises a lipid nanoparticle (LNP), a polymeric microparticle, and an oil-in-water emulsion. For example, a polynucleotide, the RNA, the cRNA or a self-replicating RNA described herein is encapsulated in, bound to or adsorbed on a LNP, a polymeric microparticle, and an oil-in-water emulsion. In one example, the polynucleotide is encapsulated in, bound to or adsorbed on a LNP, a polymeric microparticle, and an oil-in-water emulsion. In another example, the RNA is encapsulated in, bound to or adsorbed on a LNP, a polymeric microparticle, and an oil-in-water emulsion. For example, the cRNA is encapsulated in, bound to or adsorbed on a LNP, a polymeric microparticle, and an oil-in-water emulsion. For example, the self-replicating RNA is encapsulated in, bound to or adsorbed on a LNP, a polymeric microparticle, and an oil-in-water emulsion.

In one example, the present disclosure provides a pharmaceutical composition comprising a polynucleotide comprising a first nucleotide sequence encoding an antigen operably linked to a regulatory element, and a second nucleotide sequence encoding a cathelicidin operably linked to a regulatory element, wherein the composition further comprises a lipid nanoparticle (LNP). Where the use of a LNP in an immunogenic composition or pharmaceutical composition is contemplated, it will be understood that the cathelicidin polypeptides disclosed herein do not form part of the LNP. Where the use of a LNP is contemplated, it will be understood that the cathelicidin polypeptides disclosed herein do not form part of the LNP.

In an example, the polynucleotide is encapsulated in a LNP. In another example, the RNA is encapsulated in a LNP. For example, the cRNA is encapsulated in a LNP. For example, the self-replicating RNA is encapsulated in a LNP. For example, the polynucleotide is bound to a LNP. In another example, the RNA is bound to a LNP. For example, the cRNA is bound to a LNP. In another example, the self-replicating RNA is bound to a LNP. For example, the polynucleotide is adsorbed on to a LNP. In another example, the RNA is adsorbed on to a LNP. For example, the cRNA is adsorbed on to a LNP. In a further example, the self-replicating RNA is adsorbed on to a LNP. In another example, wherein each RNA is formulated together in the LNP. In another example, wherein each RNA is formulated separately in the LNP.

In one example, the RNA encoding the adjuvant and the RNA encoding the antigen are contained within the same LNP.

In one example, the composition further comprises an additional RNA encoding:
(i) one or more antigens;
(ii) one or more immune response enhancers;
(iii) one or more adjuvants; and/or
(iv) one or more targeting molecules.

In one example, the additional RNA is contained within the same LNP as the RNA encoding the adjuvant and/or the RNA encoding the antigen or is contained within a LNP separate to the RNA encoding the adjuvant and the RNA encoding the antigen.

In one example, the LNP comprises a PEG-lipid, a structural lipid and/or a neutral lipid. For example, the LNP comprises a PEG-lipid, a structural lipid and a neutral lipid. In another example, the LNP comprises a PEG-lipid, a structural lipid or a neutral lipid.

In one example, the LNP further comprises a cationic lipid. In another example, the LNP does not comprise a cationic lipid.

In one example, the pharmaceutical composition further comprises a polymeric microparticle. For example, the polynucleotide is encapsulated in a polymeric microparticle. In another example, the RNA is encapsulated in a polymeric microparticle. For example, the cRNA is encapsulated in a polymeric microparticle. For example, the self-replicating RNA is encapsulated in a polymeric microparticle. For example, the polynucleotide is bound to a polymeric microparticle. In another example, the RNA is bound to a polymeric microparticle. For example, the cRNA is bound to a polymeric microparticle. In another example, the self-replicating RNA is bound to a polymeric microparticle. For example, the polynucleotide is adsorbed on to a polymeric microparticle. In another example, the RNA is adsorbed on to a polymeric microparticle. For example, the cRNA is adsorbed on to a polymeric microparticle. In a further example, the self-replicating RNA is adsorbed on to a polymeric microparticle.

In one example, the pharmaceutical composition further comprises an oil-in-water emulsion. For example, the polynucleotide is encapsulated in an oil-in-water emulsion. In another example, the RNA is encapsulated in an oil-in-water emulsion. For example, the cRNA is encapsulated in an oil-in-water emulsion. For example, the self-replicating RNA is encapsulated in an oil-in-water emulsion. For example, the polynucleotide is bound to an oil-in-water emulsion. In another example, the RNA is bound to an oil-in-water emulsion. For example, the cRNA is bound to an oil-in-water emulsion. In another example, the self-replicating RNA is bound to an oil-in-water emulsion. In a further example, the self-replicating RNA is adsorbed on to an oil-in-water emulsion. In a further example, the self-replicating RNA is resuspended in an oil-in-water emulsion.

The present disclosure also provides an immunogenic composition or a pharmaceutical composition of the disclosure for use as a vaccine.

In one example, the polynucleotide is DNA. In one example, the disclosure provides a DNA encoding a cRNA vaccine of the disclosure. In one example, the disclosure provides a DNA encoding a self-replicating RNA vaccine of the disclosure.

In one example, the DNA is a plasmid.

The present disclosure provides a method of treating or preventing or delaying progression of a disease or condition in a subject, the method comprising administering the immunogenic composition or the pharmaceutical composition of the present disclosure to a subject in need thereof. In one example, the disclosure provides a method of treating a disease or condition in a subject, the method comprising administering the immunogenic composition or the pharmaceutical composition of the present disclosure to a subject in need thereof. In another example, the disclosure provides a method of preventing a disease or condition in a subject, the method comprising administering the immunogenic composition or the pharmaceutical composition of the present disclosure to a subject in need thereof. In a further example, the disclosure provides a method of delaying progression of a disease or condition in a subject, the method comprising administering the immunogenic composition or the pharmaceutical composition of the present disclosure to a subject in need thereof.

In one example, the present disclosure provides use of a polynucleotide of the disclosure in the manufacture of a medicament for treating or preventing or delaying progression of a disease or condition in a subject in need thereof. For example, the disclosure provides use of a polynucleotide of the disclosure in the manufacture of a medicament for treating a disease or condition in a subject in need thereof. In another example, the disclosure provides use of a polynucleotide of the disclosure in the manufacture of a medicament for preventing a disease or condition in a subject in need thereof. In a further example, the disclosure provides use of a polynucleotide of the disclosure in the manufacture of a medicament for delaying progression of a disease or condition in a subject in need thereof.

In one example, the present disclosure provides use of a RNA of the disclosure in the manufacture of a medicament for treating or preventing or delaying progression of a disease or condition in a subject in need thereof. For example, the disclosure provides use of a RNA of the disclosure in the manufacture of a medicament for treating a disease or condition in a subject in need thereof. In another example, the disclosure provides use of a RNA of the disclosure in the manufacture of a medicament for preventing a disease or condition in a subject in need thereof. In a further example, the disclosure provides use of a RNA of the disclosure in the manufacture of a medicament for delaying progression of a disease or condition in a subject in need thereof.

In one example, the present disclosure provides use of a cRNA of the disclosure in the manufacture of a medicament for treating or preventing or delaying progression of a disease or condition in a subject in need thereof. For example, the disclosure provides use of a cRNA of the disclosure in the manufacture of a medicament for treating a disease or condition in a subject in need thereof. In another example, the disclosure provides use of a cRNA of the disclosure in the manufacture of a medicament for preventing a disease or condition in a subject in need thereof. In a further example, the disclosure provides use of a cRNA of the disclosure in the manufacture of a medicament for delaying progression of a disease or condition in a subject in need thereof.

In one example, the present disclosure provides use of a self-replicating RNA of the disclosure in the manufacture of a medicament for treating or preventing or delaying progression of a disease or condition in a subject in need thereof. For example, the disclosure provides use of a self-replicating RNA of the disclosure in the manufacture of a medicament for treating a disease or condition in a subject in need thereof. In another example, the disclosure provides use of a self-replicating RNA of the disclosure in the manufacture of a medicament for preventing a disease or condition in a subject in need thereof. In a further example, the disclosure provides use of a self-replicating RNA of the disclosure in the manufacture of a medicament for delaying progression of a disease or condition in a subject in need thereof.

In another example, the present disclosure provides a polynucleotide of the disclosure for use in treating or preventing or delaying progression of a disease or condition in a subject in need thereof. In one example, the disclosure provides a RNA of the disclosure for use in treating or preventing or delaying progression of a disease or condition in a subject in need thereof. In another example, the disclosure provides a cRNA of the disclosure for use in treating or preventing or delaying progression of a disease or condition in a subject in need thereof. In yet another example, the disclosure provides a self-replicating RNA of the disclosure for use in treating or preventing or delaying progression of a disease or condition in a subject in need thereof.

In one example, the subject suffers from a disease or condition. In one example, the subject has been diagnosed as suffering from a disease or condition. In one example, the subject is receiving treatment for a disease or condition.

In an example, the present disclosure provides a method of inducing an immune response in a subject, the method comprising administering a RNA disclosed herein (e.g., self-replicating RNA), a pharmaceutical composition disclosed herein, an immunogenic composition disclosed herein or a vaccine disclosed herein to a subject in need thereof.

In an example, the present disclosure provides use of a RNA disclosed herein (e.g., self-replicating RNA), a pharmaceutical composition disclosed herein, an immunogenic composition disclosed herein or a vaccine disclosed herein in the manufacture of a medicament for inducing an immune response in a subject in need thereof.

In an example, the present disclosure provides a RNA disclosed herein (e.g., self-replicating RNA), a pharmaceutical composition disclosed herein, an immunogenic composition disclosed herein or a vaccine disclosed herein for use in inducing an immune response in a subject in need thereof.

In one example, the composition induces a humoral immune response in the subject. For example, the humoral immune response is an antibody-mediated immune response. For example, production of neutralizing antibodies. In another example, the composition induces a cell-mediated immune response. For example, the cell-mediated immune response includes activation of antigen-specific cytotoxic T cells. For example, the T cells are CD4 T cells and/or CD8 T cells. In one example, the T cells are CD4 T cells. In another example the T cells are CD8 T cells. In a further example, the T cells are CD4 and CD8 T cells.

In one example, administration of a RNA disclosed herein (e.g., self-replicating RNA), a pharmaceutical composition disclosed herein, an immunogenic composition disclosed herein or a vaccine of the present disclosure induces a CD4 T cell mediated immune response.

In one example, administration of a RNA disclosed herein (e.g., self-replicating RNA), a pharmaceutical composition, an immunogenic composition disclosed herein or a vaccine of the present disclosure induces a CD8 T cell mediated immune response.

In one example, administration of a RNA disclosed herein (e.g., self-replicating RNA), a pharmaceutical composition disclosed herein, an immunogenic composition disclosed herein or a vaccine of the present disclosure induces a CD4 and a CD8 T cell mediated immune response.

In one example, the CD4 T cell mediated immune response is a Th0, a Th1 and/or a Th2 response. For example, the CD4 T cell mediated immune response is a Th0 response. In another example, the CD4 T cell mediated immune response is a Th1 response. In a further example, the CD4 T cell mediated immune response is a Th2 response. In one example, the CD4 T cell mediated immune response is a Th0 and Th1 response. In another example, the CD4 T cell mediated immune response is a Th0 and Th2 response. In a further example, the CD4 T cell mediated immune response is a Th1 and Th2 response. In another example, the CD4 T cell mediated immune response is a Th0, Th1 and Th2 response.

In one example, the Th0 response cytokines express interleukin 2 (IL2+) and/or tumor necrosis factor alpha (TNFa+); and/or are negative for interferon gamma (IFNg−), IL5− and/or IL13−. For example, the cytokine is IL2+. In another example, the cytokine is TNFa+. In one example, the cytokine is IFNg−. In another example, the cytokine is IL5−. In a further example, the cytokine is IL13−.

In one example, the Th1 response cytokines express interferon gamma (IFNg+); and/or are negative for IL5− and/or IL13−. For example, the cytokine is IFNg+. In another example, the cytokine is IL5−. In a further example, the cytokine is IL13−.

In one example, the Th2 response cytokines express IL5+ and/or IL13+; and/or are negative for IFNg. For example, the cytokine is IL5+. In a further example, the cytokine is IL13+. For example, the cytokine is IFNg−.

In an example, the present disclosure provides a method for reducing viral load in a subject having a viral infection comprising administering a RNA disclosed herein (e.g., self-replicating RNA), a pharmaceutical composition disclosed herein, an immunogenic composition disclosed herein or a vaccine disclosed herein to a subject having a viral infection.

In an example, the present disclosure provides use of a RNA disclosed herein (e.g., self-replicating RNA), a pharmaceutical composition disclosed herein, an immunogenic composition disclosed herein or a vaccine disclosed herein in the preparation of a medicament for reducing viral load in a subject having a viral infection.

In an example, the present disclosure provides a RNA disclosed herein (e.g., self-replicating RNA), a pharmaceutical composition disclosed herein, an immunogenic composition disclosed herein or a vaccine disclosed herein for use in reducing viral load in a subject having a viral infection.

In an example, the subject is a human of 18 years of age or older. In another example, the subject is a human of any age, e.g., from about 1 month to 100 years old, e.g., from about 2 months to about 80 years old, from about 6 months of age to about 3 years old, from about 3 years to about 18 years old, from about 12 years to about 18 years old, from about 18 years to about 55 years old, from about 50 years to about 75 years old, from about 40 years to about 65 years old. In another example, the subject is a human from 2 years of age. In another example, subject is a human from 18 years of age, a human from 30 years of age, a human from 40 years of age, a human from 50 years of age, a human from 60 years of age, a human from 70 years of age, a human from 80 years of age or a human from about 90 years of age. In another example, the subject is less than 2 years of age, less than 18 months of age, less than 12 months of age, less than 6 months of age or less than 3 months of age.

In an example, a composition or vaccine described herein is administered in a one dose regimen. In another example, the composition is administered in a two, three or four dose regimen, wherein the doses are administered about 1, 2 or 3 months apart.

The present disclosure also provides a polynucleotide that encodes the self-replicating RNA of the present disclosure. For example, the polynucleotide is a recombinant DNA. In one example, the recombinant DNA is a plasmid. In one example, the plasmid comprises a sequence set forth in SEQ ID NO: 32.

The present disclosure also provides a kit comprising at least one self-replicating RNA of the disclosure, optionally in a delivery system and/or a pharmaceutically acceptable carrier or diluent, packaged with instructions for use in treating or preventing or delaying progression of a disease or disorder in a subject.

The present disclosure also provides a kit comprising at least one self-replicating RNA of the disclosure, optionally in a delivery system and/or a pharmaceutically acceptable carrier or diluent, packaged with instructions to administer the RNA to a subject who is suffering from, or at risk of suffering from, a disease or disorder.

In one example, the self-replicating RNA, the RNA, the immunogenic composition or the pharmaceutical composition of the disclosure is supplied in a vial. In another example, the RNA, the self-replicating RNA, the immunogenic composition or the pharmaceutical composition of the disclosure is supplied in a syringe.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

KEY TO SEQUENCE LISTING

| Sequence | SEQ ID NO |
|---|---|
| mRNA sequence of Homo sapiens cathelicidin | 1 |
| DNA sequence of Homo sapiens cathelicidin | 2 |
| Amino acid sequence of Homo sapiens cathelicidin | 3 |
| mRNA sequence of Homo sapiens C-terminal truncated cathelicidin | 4 |
| (hCAP18), cleavage between nucleotide position 103 and 104 | |
| DNA sequence of Homo sapiens C-terminal truncated cathelicidin | 5 |
| (hCAP18), cleavage between nucleotide position 103 and 104 | |
| Amino acid sequence of Homo sapiens C-terminal truncated | 6 |
| cathelicidin, cleavage between nucleotide position 103 and 104 | |
| mRNA sequence of Mus Musculus cathelicidin homolog of human | 7 |
| LL37 | |
| DNA sequence of Mus Musculus cathelicidin homolog of human | 8 |
| LL37 | |
| Amino acid sequence of Mus Musculus cathelicidin homolog of | 9 |
| human LL37 | |
| mRNA sequence that encodes a 38 amino acid mouse cathelicidin | 10 |
| homolog of human LL37 | |
| DNA sequence that encodes a 38 amino acid mouse cathelicidin | 11 |
| homolog of human LL37 | |
| Amino acid sequence that encodes a 38 amino acid mouse | 12 |
| cathelicidin homolog of human LL37 | |
| Amino acid sequence of the human LL37 sequence in reverse | 13 |
| orientation. | |
| Nucleotide sequence of alphavirus native subgenomic promoter | 14 |
| Nucleotide sequence of extended subgenomic promoter (v2) | 15 |
| Nucleotide sequence of extended subgenomic promoter (v3) | 16 |
| Nucleotide sequence of wild-type EMCV IRES | 17 |
| Nucleotide sequence of extended subgenomic promoter (v4) | 18 |
| Nucleotide sequence of a Kozak consensus sequence | 19 |
| Nucleotide sequence of a Kozak consensus sequence | 20 |
| Nucleotide sequence of an interrupting linker | 21 |
| Nucleotide sequence of a GC-rich element | 22 |
| Nucleotide sequence of a GC-rich element | 23 |
| Nucleotide sequence of a GC-rich element | 24 |
| Nucleotide sequence of a histone stem loop | 25 |
| Nucleotide sequence of 5′UTR of VEEV | 26 |
| Nucleotide sequence of 3′UTR of SINV | 27 |
| Nucleotide sequence of extended subgenomic promoter | 28 |
| Poly-A sequence | 29 |
| DNA sequence of self-replicating VEEV mRNA-H5 | 30 |
| RNA sequence of self-replicating VEEV mRNA-H5 | 31 |
| Plasmid DNA of VEEV self-replicating mRNA-H5 | 32 |
| Nucleotide sequence of a subgenomic promoter | 33 |

### DETAILED DESCRIPTION

### General

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter.

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific examples described herein, which are intended for the purpose of exemplification only. Functionally-equivalent products, compositions and methods are clearly within the scope of the present disclosure.

Any example of the present disclosure herein shall be taken to apply mutatis mutandis to any other example of the disclosure unless specifically stated otherwise. Stated another way, any specific example of the present disclosure may be combined with any other specific example of the disclosure (except where mutually exclusive).

Any example of the present disclosure disclosing a specific feature or group of features or method or method steps will be taken to provide explicit support for disclaiming the specific feature or group of features or method or method steps.

Unless specifically defined otherwise, all technical and scientific terms used herein shall be taken to have the same meaning as commonly understood by one of ordinary skill in the art (for example, in cell culture, molecular genetics, immunology, immunohistochemistry, protein chemistry, and biochemistry).

Unless otherwise indicated, the recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al. Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The term “and/or”, e.g., “X and/or Y” shall be understood to mean either “X and Y” or “X or Y” and shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used herein the term “derived from” shall be taken to indicate that a specified integer may be obtained from a particular source albeit not necessarily directly from that source. Similarly, the term “based on” shall be taken to indicate that a specified integer may be developed or used from a particular source albeit not necessarily directly from that source.

### Selected Definitions

As used herein, the term “monocistronic” in reference to the polynucleotide, RNA, cRNA and/or self-replicating RNA, refers to a RNA that encodes one polypeptide.

As used herein, the term “multicistronic” (also known as “polycistronic”) in reference to the polynucleotide, RNA, cRNA and/or self-replicating RNA, refers to a RNA that encodes two or more polypeptides. The term encompasses “bicistronic” (or “dicistronic”; i.e., encoding two polypeptides) and “tricistronic” (i.e., encoding three polypeptides) molecules. By “bicistronic” is meant a single nucleic acid that is capable of encoding two distinct polypeptides from different regions of the nucleic acid.

As used herein, the term “conventional mRNA” or “cRNA” or “non-amplifying RNA” refers to a construct that allows expression of heterologous RNA and/or proteins but the RNA that cannot amplify in host cells.

As used herein, the term “self-replicating RNA” refers to a construct based on a RNA virus that has been engineered to allow expression of heterologous mRNA and proteins. Self-replicating RNA (e.g., in the form of naked RNA) can amplify in host cells leading to expression of the desired gene product in the host cell.

The term “naked” as used herein refers to nucleic acids that are substantially free of other macromolecules, such as lipids, polymers and proteins. A “naked” nucleic acid, such as a self-replicating RNA, is not formulated with other macromolecules to improve cellular uptake. Accordingly, a naked nucleic acid is not encapsulated in, absorbed on, or bound to a lipid nanoparticle (LNP), a liposome, a polymeric microparticle or an oil-in-water emulsion.

As used herein, the term “nucleotide sequence” or “nucleic acid sequence” will be understood to mean a series of contiguous nucleotides (or bases) covalently linked to a phosphodiester backbone. By convention, sequences are presented from the 5′ end to the 3′ end, unless otherwise specified. To facilitate a clear description of the nucleic acids, particular sequence components are referred to as e.g., a “first nucleotide sequence” and a “second nucleotide sequence”. It is to be understood that the first and second sequences can appear in any desired order or orientation, unless otherwise specified, and that no particular order or orientation is intended by the words “first”, “second” etc.

As used herein, the term “antigen” refers to a molecule or structure containing one or more epitopes that induce, elicit, augment or boost a cellular and/or humoral immune response. Antigens can include, for example, proteins and peptides from a pathogen such as a virus, bacteria, fungus, protozoan, plant or from a tumour.

As used herein the term “adjuvant” refers to a compound that, when used in combination with a specific immunogen in a formulation, augments or otherwise alters or modifies the resultant immune response. Modification of the immune response may include intensification or broadening the specificity of either or both antibody and cellular immune responses. Modification of the immune response can also mean decreasing or suppressing certain antigen-specific immune responses.

As used herein, the term “operably linked to” means positioning a subgenomic promoter or regulatory element (e.g., an IRES) relative to a nucleic acid such that expression of the nucleic acid is controlled or regulated by the element. For example, a subgenomic promoter can be operably linked to numerous nucleic acids, e.g., through another regulatory element, such as an internal ribosome entry site (IRES).

As used herein, the term “subgenomic promoter” or “SG promoter” (also known as ‘junction region’ promoter) refers to a promoter that directs the expression of a heterologous nucleotide sequence, regulating protein expression.

As used herein, the term “internal ribosome entry site” or “IRES” refers to a sequence of nucleotides within a mRNA to which a ribosome or a component thereof, e.g., a 40S subunit of a ribosome, is capable of binding. An IRES need not necessarily comprise nucleic acid that induces translation of a mRNA (e.g., a start codon; AUG).

The term “polypeptide” or “polypeptide chain” will be understood to mean a series of contiguous amino acids linked by peptide bonds. For example, a protein shall be taken to include a single polypeptide chain i.e., a series of contiguous amino acids linked by peptide bonds or a series of polypeptide chains covalently or non-covalently linked to one another (i.e., a polypeptide complex). The series of polypeptide chains can be covalently linked using a suitable chemical or a disulfide bond. Examples of non-covalent bonds include hydrogen bonds, ionic bonds, Van der Waals forces, and hydrophobic interactions.

The term “recombinant” shall be understood to mean the product of artificial genetic recombination.

As used herein the term “substantially the same” in reference to the level of expression is meant that the first and second antigens (at least) have a level of expression within about 10% or less of each other unless the context implies otherwise.

As used herein, the terms “disease”, “disorder” or “condition” refers to a disruption of or interference with normal function, and is not to be limited to any specific condition, and will include diseases or disorders.

As used herein, a subject “at risk” of having developed or developing a disease or condition may have or may not have detectable disease or symptoms of the disease or condition, and may have or may not have displayed detectable disease or symptoms of the disease or condition prior to the treatment according to the present disclosure. “At risk” denotes that a subject has one or more risk factors, which are measurable parameters that correlate with development of the disease or condition, as known in the art and/or described herein.

As used herein, the terms “treatment” or “treating” of a subject includes the application or administration of a RNA, polynucleotide or composition of the disclosure to a subject (or application or administration of a RNA, polynucleotide or composition of the disclosure to a cell or tissue from a subject) with the purpose of delaying, slowing, stabilizing, curing, healing, alleviating, relieving, altering, remedying, less worsening, ameliorating, improving, or affecting the disease or condition, the symptom of the disease or condition, or the risk of (or susceptibility to) the disease or condition. The term “treating” refers to any indication of success in the treatment or amelioration of an injury, pathology or condition, including any objective or subjective parameter such as abatement; remission; lessening of the rate of worsening; lessening severity of the disease; stabilization, diminishing of symptoms or making the injury, pathology or condition more tolerable to the subject; slowing in the rate of degeneration or decline; or making the final point of degeneration less debilitating.

As used herein, “preventing” or “prevention” is intended to refer to at least the reduction of likelihood of the risk of (or susceptibility to) acquiring a disease or disorder (i.e., causing at least one of the clinical symptoms of the disease not to develop in a patient that may be exposed to or predisposed to the disease but does not yet experience or display symptoms of the disease). Biological and physiological parameters for identifying such patients are provided herein and are also well known by physicians.

As used herein, the phrase “delaying progression of” includes reducing or slowing down the progression of the disease or condition in an individual and/or at least one symptom of a disease or condition.

An “effective amount” refers to at least an amount effective, at dosages and for periods of time necessary, to achieve the desired result. For example, the desired result may be a therapeutic or prophylactic result. An effective amount can be provided in one or more administrations. In some examples of the present disclosure, the term “effective amount” is meant an amount necessary to effect treatment of a disease or condition as hereinbefore described. In some examples of the present disclosure, the term “effective amount” is meant an amount necessary to effect a change associated with a disease or condition as hereinbefore described. The effective amount may vary according to the disease or condition to be treated or factor to be altered and also according to the weight, age, racial background, sex, health and/or physical condition and other factors relevant to the mammal being treated. Typically, the effective amount will fall within a relatively broad range (e.g. a “dosage” range) that can be determined through routine trial and experimentation by a medical practitioner. Accordingly, this term is not to be construed to limit the disclosure to a specific quantity, e.g., weight or number of RNA. The effective amount can be administered in a single dose or in a dose repeated once or several times over a treatment period.

A “therapeutically effective amount” is at least the minimum concentration required to effect a measurable improvement of a particular disease or condition. A therapeutically effective amount herein may vary according to factors such as the disease state, age, sex, and weight of the patient, and the ability of the RNA of the present disclosure to elicit a desired response in the individual. A therapeutically effective amount is also one in which any toxic or detrimental effects of the RNA are outweighed by the therapeutically beneficial effects.

As used herein, the term “prophylactically effective amount” shall be taken to mean a sufficient quantity of the RNA of the disclosure to prevent or inhibit or delay the onset of one or more detectable symptoms of a disease or disorder as described herein.

A “subject” can be any animal that is susceptible to a disease or condition described herein. A subject can be a mammal and in particular embodiments is a human, which can be an infant, a child, an adult or an elderly adult. A “subject at risk of a disease or condition” is any subject who may be or has been exposed to the disease or condition. “Subject” includes any human or non-human animal. Thus, in addition to being useful for human treatment, a RNA of the present disclosure may also be useful for veterinary treatment of mammals, including companion animals and farm animals, such as, but not limited to dogs, cats, horses, cows, sheep, and pigs.

As used herein, the term “lipid nanoparticle” or “LNP” shall be understood to refer to lipid-based particles having at least one dimension on the order of nanometers (e.g., 1-1,000 nm) and which comprises a polynucleotide, RNA, mRNA or self-amplifying RNA described herein. In embodiments, LNPs are formulated in a composition for delivery of a polynucleotide to a desired target such as a cell, tissue, organ, tumor, and the like. For example, the lipid nanoparticle or LNP any lipid composition, may be selected from, but not limited to, liposomes or vesicles, where an aqueous volume is encapsulated by amphipathic lipid bilayers (e.g., single; unilamellar or multiple; multilamellar), micelle-like lipid nanoparticles having a non-aqueous core and solid lipid nanoparticles, wherein solid lipid nanoparticles lack lipid bilayers.

### Polynucleotides

As used herein, the term “polynucleotide” refers a molecular chain of nucleotides chemically bonded by a series of ester linakges between the phosphoryl group of one nucleotide and the hydroxyl group of the sugar in an adjacent nucleotide. In one example, the polynucleotide is a DNA. In one example, the polynucleotide is a RNA, e.g., mRNA. For example, the mRNA is a conventional mRNA (cRNA) or a self-replicating RNA.

As used herein, the term “fragment” refers to a portion of a nucleotide sequence or polypeptide of a reference nucleotide sequence or polypeptide disclosed herein which maintains a defined activity of the full length nucleotide sequence or polypeptide. For example, a cathelicidin fragment disclosed herein will be understood to retain its function as an adjuvant.

By way of non-limiting example, where the conventional mRNA, self-replicating RNA comprises a polynucleotide encoding more than one antigen, said more than one antigen may be expressed by a monocistronic polynucleotide, or each of said antigens may be expressed by polycistronic (or multicistronic) polynucleotides. In another example, a RNA, conventional RNA, self-replicating RNA or polynucleotide disclosed herein may be a monocistronic RNA, conventional RNA, self-replicating RNA or polynucleotide; or it may be a multicistronic RNA, conventional RNA, self-replicating RNA or polynucleotide.

As used herein, the term “variant” refers to a nucleotide sequence with one or more substitutions, insertions, deletions and/or other modifications compared to the unmodified sequence. It will be apparent to the skilled person that any variant described herein will have the same or similar expression of the encoded protein. For example, the variant is a functional variant. Exemplary modifications to the nucleotide sequence and/or polypeptide will be apparent to the skilled person and/or described herein.

In one example, a modification is a chemical modification of one or more nucleotide(s) of the nucleotide sequence. For example, at least one naturally occurring nucleotide of the polynucleotide is replaced with a chemically modified nucleotide (e.g. pseudouridine (ψ), and 1-methylpseudouridine (m1ψ)).

In one example, the modification comprises increasing the G/C content of the nucleotide sequence.

In one example, the modification comprises codon optimization of the nucleotide sequence.

In one example, the substitution is a conservative substitution. A skilled person will appreciate that a conservative substitution with reference to a polypeptide involves replacement of an amino acid in the polypeptide with a different amino acid with similar biochemical properties (e.g. charge, hydrophobicity and size). In one example, the substitution is a non-conservative substitution.

As used herein, the term “encode”, “encodes” or “encoding” refers to a region of a polynucleotide capable of undergoing translation into a polypeptide.

The polynucleotide of the present disclosure includes DNA and RNA (e.g. mRNA).

### Deoxyribonucleic Acid (DNA)

In one example, the polynucleotide is a DNA (e.g. DNA vector).

It will be apparent to the skilled person that a DNA of the present disclosure further comprises an endonuclease restriction site at the 3′ end of the 3′UTR. The skilled person will appreciate that endonuclease restriction site allows for the insertion of one or more nucleotide sequence(s) (e.g. encoding an antigen of interest, a fragment and/or a variant thereof) without disrupting the remainder of the DNA.

As used herein, the term “restriction endonuclease site” refers to a sequence of DNA that binds to a restriction endonuclease. Typically, the restriction endonuclease site is short sequence (e.g. of approximately 4-8 base pairs) recognised and cleaved by the restriction endonuclease.

As used herein, the term “restriction enzymes” or “restriction endonucleases” refers to a class of enzyme that occur naturally in bacteria and in some viruses.

Restriction endonuclease bind specifically to and cleave double-stranded DNA at specific sites within or adjacent to a restriction endonuclease site. Exemplary restriction endonuclease include, for example, BciVI (Bful), Bcul (Spel), EcoRI, Aatll, AgeI (BshTI), Apal, BamHI, BglII, Blpl (Bpul102I), BsrGI (Bspl407), Clal (Bsul5I), EcoRI, EcoRV (Eco32I), Eam1104I (Earl), Hindlll, Kpnl, Mlul, Ncol, Ndel, Nhel, Notl, Nsil, Mph1103I), Pstl, Pvul, Pvull, SacI, SalI, ScaI, SpeI, Xbal, Xhol, Sacll (Cfr42I) and Xbal.

In one example, the present disclosure provides a transcribable polynucleotide comprising the first nucleotide sequence encoding a first antigen of interest; and/or a second nucleotide sequence encoding an adjuvant operably linked to a regulatory element such as a SG promoter and an IRES. For example, the polynucleotide is the DNA plasmid comprising the first and second nucleotide sequences.

In one example, the DNA comprises a nucleotide sequence comprising a restriction endonuclease site located 3′ of the 3′UTR. The presence of the restriction endonuclease site located 3′ of the 3′UTR allows for production of a linearised DNA. Linearisation of DNA ensures defined termination of in vitro transcribed DNA to produce mRNA.

### Ribonucleic Acid (RNA)

In one example, the polynucleotide is a RNA comprising a first nucleotide sequence encoding an antigen operably linked to a regulatory element, and a second nucleotide sequence encoding an adjuvant operably linked to a regulatory element, wherein the adjuvant is a cathelicidin or a fragment thereof.

The RNA of the present disclosure may be a mRNA, which encompasses a non-replicating mRNA (also referred to as conventional mRNA (cRNA) or non-amplifying), in addition to a self-replicating RNA (also known as self-amplifying RNA or sa-mRNA).

### Conventional (Non-Replicating) RNA

In one example, the polynucleotide is a cRNA comprising a first nucleotide sequence encoding an antigen operably linked to a regulatory element, and a second nucleotide sequence encoding an adjuvant operably linked to a regulatory element, wherein the adjuvant is a cathelicidin or a fragment thereof.

In an example, the cRNA of the present disclosure comprises in order from 5′ to 3′: a 5′ cap structure, a 5′-UTR, a fragment and/or a variant thereof, a first nucleotide sequence encoding a first antigen of interest, a second nucleotide sequence encoding an adjuvant, a 3′-UTR and a 3′ tailing sequence (e.g. a polyadenylation signal or one or more poly-A tails), wherein the adjuvant is a cathelicidin. The cRNA of the present disclosure may further comprise a translation internal ribosome entry site (e.g. Kozak consensus sequence or IRES) operably linked to the antigen of interest or the adjuvant.

### Self-Replicating RNA

The present disclosure provides a self-replicating RNA (also known as a replicon or self-amplifying RNA) comprising a first nucleotide sequence encoding an antigen operably linked to a regulatory element, and a second nucleotide sequence encoding an adjuvant operably linked to a regulatory element, wherein the adjuvant is a cathelicidin or a fragment thereof.

The skilled person will understand that the self-replicating RNA of the present disclosure is based on the genomic RNA of RNA viruses. The RNA should be positive (+)-stranded so that it can be directly translated after delivery to a cell without the need for intervening replication steps (e.g., reverse transcription). Translation of the RNA results in the production of non-structural proteins (NSPs) which combine to form a replicase complex (i.e., a RNA-dependent RNA polymerase). The complex then amplifies the original RNA, producing both antisense and sense transcripts, resulting in production of multiple daughter RNAs which may subsequently be translated and transcribed, enhancing overall protein expression.

In one example, the self-replicating RNA of the present disclosure comprises the non-structural proteins of the RNA virus, the 5′ and 3′ untranslated regions (UTRs) and the native subgenomic promoter.

In one example, the self-replicating RNA comprises one or more non-structural proteins of the RNA virus. For example, the RNA comprises at least one or more genes selected from the group consisting of a viral replicase (or viral polymerase), a viral protease, a viral helicase and other non-structural viral proteins. For example, the self-replicating RNA comprises a viral replicase (or viral polymerase).

In another example, the self-replicating RNA comprises a 5′- and a 3′-end UTR of the RNA virus. It will be apparent to the skilled person that the terms 5′ and a 3′UTR also encompasses the terms 5′ and 3′ conserved sequence elements (CSE). In one example, the self-replicating RNA comprises a 5′- and a 3′-end CSE.

The self-replicating RNA of the present disclosure cannot induce production of infectious viral particles. For example, the self-replicating RNA of the present disclosure does not comprise viral genes encoding structural proteins necessary for production of viral particles.

In one example, the self-replicating RNA is derived from or based on an alphavirus. Suitable alphaviruses will be apparent to the skilled person and/or described herein.

In another example, the self-replicating RNA is derived from or based on a virus other than an alphavirus, for example, a positive-stranded RNA virus. Positive-stranded RNA viruses suitable for use in the present disclosure will be apparent to the skilled person and include, for example, a picornavirus, a flavivirus, a rubivirus, a pestivirus, a hepacivirus, a calicivirus, or a coronavirus.

### Alphavirus

In one example, the self-replicating RNA of the present disclosure is derived from (or based on) an alphavirus.

Alphaviruses are the sole genus in the Togaviridae family and are an enveloped virus with a positive-sense, single-stranded RNA genome. The skilled person will understand that the alphavirus genome comprises two open reading frames (ORFs), non-structural and structural. The first ORF encodes four non-structural proteins (NSP1, NSP2, NSP3 and NSP4) necessary for transcription and replication of viral RNA. The second encodes three structural proteins: the core nucleocapsid protein C, and the envelope proteins P62 and E1, which associate as a heterodimer. The viral membrane-anchored surface glycoproteins are responsible for receptor recognition and entry into target cells through membrane fusion.

In one example, the self-replicating RNA of the present disclosure comprises a viral replicase (or viral polymerase). For example, the viral replicase is an alphavirus replicase, such as an alphavirus protein NSP4.

In one example, the self-replicating RNA of the present disclosure does not encode one or more alphavirus structural proteins (e.g., capsid and/or envelope glycoproteins). For example, the self-replicating RNA is unable to produce RNA-containing alphavirus virions (i.e., infectious viral particles).

In one example, the self-replicating RNA comprises a native alphavirus SG promoter. For example, the native alphavirus SG promoter is a minimal SG promoter (i.e., the minimal sequence required for initiation of transcription) and comprises a sequence set forth in SEQ ID NO: 14.

The skilled person will be aware of alphaviruses suitable for use in the present disclosure. Exemplary alphaviruses include, but are not limited to, Venezuelan equine encephalitis virus (VEE; e.g., Trinidad donkey, TC83CR), Semliki Forest virus (SFV), Sindbis virus (SIN), Ross River virus, Western equine encephalitis virus, Eastern equine encephalitis virus, Chikungunya virus, S.A. AR86 virus, Everglades virus, Mucambo virus, Barmah Forest virus, Middelburg virus, Pixuna virus, O'nyong-nyong virus, Getah virus, Sagiyama virus, Bebaru virus, Mayaro virus, Una virus, Aura virus, Whataroa virus, Banbanki virus, Kyzylagach virus, Highlands J virus, Fort Morgan virus, Ndumu virus, and Buggy Creek virus. The term alphavirus may also include chimeric alphaviruses (e.g., as described by Perri et al, (2003) J. Virol. 77(19): 10394-403) that contain genome sequences from more than one alphavirus.

### Regulatory Elements

The present disclosure provides a polynucleotide comprising a first nucleotide sequence encoding an antigen operably linked to a regulatory element, and a second nucleotide sequence encoding an adjuvant operably linked to a regulatory element, wherein the adjuvant is a cathelicidin or a fragment thereof.

In an example, the regulatory element is selected from the group consisting of a subgenomic (SG) promoter and an internal ribosome entry site (IRES) and a Kozac consensus sequence or a combination thereof. In an example, the regulatory element is a SG promoter.

### Kozak Consensus Sequence

As used herein, the term “Kozak consensus sequence” refers to a nucleotide sequence identified in eukaryotic genes that facilitates the translation of the gene by containing a start codon (also referred to as a translation initiation codon) which is recognised by a ribosome.

Exemplary Kozak consensus sequence are known in the art and/or described herein. In one example, the Kozak consensus sequence is set forth in SEQ ID NO: 19 (GCCACC). In another example, the Kozak consensus sequence is set forth in SEQ ID NO: 20 (ACCATGG). In one example, the Kozak consensus sequence is set forth in SEQ ID NO: 20 (ACCATGG). In another example, the Kozak consensus sequence is ACCATG.

### Subgenomic Promoters

SG promoters (also known as ‘junction region’ promoters) suitable for use in the present disclosure will be apparent to the skilled person and/or are described herein.

In one example, the SG promoter is derived from or based on an alphavirus SG promoter. For example, the SG promoter is a native alphavirus SG promoter. In one example, the native SG promoter is a minimal SG promoter. For example, the minimal SG promoter is the minimal sequence required for initiation of transcription. Exemplary minimal SG promoter sequences are encoded by SEQ ID NO: 14 and SEQ ID NO: 33.

In one example, the native SG promoter is an extended SG promoter. For example, the extended SG promoter is a minimal SG promoter extended at the 5′ end with nucleotides occurring in a sequence encoding a non-structural protein (e.g., NSP4) of the RNA virus (e.g., an alphavirus). In one example, the extended SG promoter is a minimal SG promoter extended at the 5′ end with nucleotides occurring in a sequence encoding an alphavirus NSP4.

In one example, the SG promoter is extended at the 5′ end by about 31 nucleotides occurring in a sequence encoding a non-structural protein (e.g., an alphavirus NSP4). In one example, the extended SG promoter is encoded by a sequence set forth in SEQ ID NO: 14 extended at the 5′ end by 31 nucleotides occurring in a sequence encoding a non-structural protein (e.g., an alphavirus NSP4). For example, the extended SG promoter is no more than 80 nucleotides in length. In one example, the extended SG promoter is encoded by a sequence comprising or consisting of nucleotides 22 to 101 of SEQ ID NO: 18. In another example, the extended SG promoter is encoded by a sequence comprising or consisting of a sequence set forth in SEQ ID NO: 16.

In one example, the extended SG promoter comprises a repeat sequence corresponding to nucleotides 66 to 75 of SEQ ID NO: 18. For example, the extended SG promoter is encoded by a sequence comprising nucleotides 50 to 75 of SEQ ID NO: 18 and nucleotides 66 to 101 of SEQ ID NO: 18. For example, the extended SG promoter is encoded by a sequence set forth in SEQ ID NO: 28.

In one example, the polynucleotide of the disclosure comprises a SG promoter from any alphavirus. For example, the RNA of the disclosure (e.g., cRNA or self-replicating RNA) comprises a SG promoter from any alphavirus.

In one example, the self-replicating RNA comprises a SG promoter from any alphavirus.

In an example, a polynucleotide of the present disclosure comprises two or more nucleotide sequences encoding two or more antigens of interest and a nucleotide sequence encoding an adjuvant, wherein the adjuvant is a cathelicidin. In one example, the two or more nucleotide sequences are each operaby linked to SG promoters. When two or more SG promoters are present in the RNA of the present disclosure, the promoters can be the same or different. For example, the two or more SG promoters are derived from the same alphavirus. In another example, the two or more SG promoters are derived from different alphaviruses.

When two or more SG promoters are present in the self-replicating RNA of the present disclosure, the promoters can be the same or different. For example, the two or more SG promoters are derived from the same alphavirus. In another example, the two or more SG promoters are derived from different alphaviruses.

In another example, where the polynucleotide of the present disclosure comprises two or more nucleotide sequences encoding two or more antigens of interest, the two or more nucleotide sequences may be driven by the same promoter or by two or more promoters, which themselves may comprise the same sequence or a different sequence.

### Internal Ribosomal Entry Site (IRES)

IRES sequences suitable for use in accordance with the present disclosure will be apparent to the skilled person and/or are described herein.

In one example, the IRES is derived from encephalomyocarditis virus (EMCV). For example, the IRES is a wild-type IRES from EMCV.

In one example, the IRES is derived from a fibroblast growth factor 1A (FGF1A) IRES.

In addition, synthetic IRES elements have been described, which can be designed, according to methods know in the art to mimic the function of naturally occurring IRES elements (see Chappell, S A et al. Proc. Natl Acad. Sci. USA (2000) 97(4): 1536-41).

In one example, the IRES is an IRES from encephalomyocarditis virus (EMCV), poliovirus (PV), human enterovirus, foot-and-mouth disease virus (FMDV), hepatitis C virus (HCV), classical swine fever virus (CSFV), murine leukemia virus (MLV), simian immunodeficiency virus (SIV), Eukaryotic translation initiation factor 4G (elF4G), Death-associated protein 5 (DAP5), cellular Myc (c-Myc), NF-κB-repressing factor (NRF), vascular endothelial growth factor (VEGF), fibroblast growth factor (FGF-2), platelet-derived growth factor B (PDGF B), Antennapedia, X-linked inhibitor of apoptosis (XIAP or Apaf-1), immunoglobulin heavy-chain binding protein BiP, or fibroblast growth factor 1a (FGF1A), GTX, or a combination thereof.

In one example, the IRES is a wild-type IRES derived from encephalomyocarditis virus (EMCV). For example, the wild-type EMCV IRES comprises a sequence set forth in SEQ ID NO: 17.

### 5′ Untranslated Region (5′-UTR)

In an example, a polynucleotide described herein comprise a 5′-untranslated region (5′-UTR), such as the 5′ UTR of an RNA virus.

As used herein, the term “5′-untranslated region” or “5′-UTR” refers to a non-coding region of an mRNA located at the 5′ end of the translation initiation sequence (AUG).

Exemplary 5′-UTRs include, for example, 5′-UTR of haptoglobin (HP), fibrinogen beta chain (FGB), haptoglobin-related protein (HPR), albumin (ALB), complement component 3 (C3), fibrinogen alpha chain (FGA), alpha 6 collagen (Col6A), alpha-1-antitrypsin (SERPINA1), alpha-1-antichymotrypsin (SERPINA3) a fragment and/or a variant thereof.

In one example, the 5′UTR is a 5′UTR of a Venezuelan equine encephalitis virus (VEEV) or modified forms thereof. For example, the 5′UTR comprises a sequence set forth in SEQ ID NO: 26.

In one example, the 5′UTR comprises at least one microRNA binding site, an AU rich element (ARE), a GC-rich element, a stem loop, and combinations thereof.

microRNA Binding Site

As used herein, the term “microRNA binding site” refers to a sequence within a polynucleotide (e.g. within a DNA or RNA transcript) that has sufficient complementarity to all or one region of a miRNA to interact, associate or bind to the microRNA (miRNA).

As used herein, the term “microRNA” or “miRNA” refers to 19-25 nucleotide long non-coding RNAs that bind to the 5′-UTR of polynucleotides and down-regulate gene expression (e.g. by inhibiting translation). The presence of microRNA binding site(s) in the 5′UTR can function to inhibit translation of the 5′-UTR.

Suitable miRNA binding sites for use in the present disclosure will be apparent to the skilled person and/or described herein.

In one example, the miRNA binding site comprises a binding site for tissue specific microRNA or those regulating biological processes. For example, miRNA of the liver (miR-122), muscle (miR-133, miR-206, miR-208), endothelial cells (miR-17-92, miR-126), myeloid cells (miR-142-3p, miR-142-5p, miR-16, miR-21, miR-223, miR-24, miR-27), adipose tissue (let-7, miR-30c), heart (miR-id, miR-149), kidney (miR-192, miR-194, miR-204), and lung epithelial cells (let-7, miR-133, miR-126). For example, microRNA that regulate biological processes such as angiogenesis (miR-132). Further exemplifying miRNA and miRNA binding sites are disclosed in U.S. patent application Ser. No. 14/043,927.

### AU Rich Element (ARE)

As used herein, the term “AU rich element (ARE)” or “AU rich elements (AREs)” refers to a region of a nucleotide sequence comprising stretches of Adeonisine (A) and Uridine (U). Exemplary AREs include, for example, ARE from cytoplasmic myc (c-myc), myoblast determination protein 1 (myoD), c-Jun, Myogenin, granulocyte-macrophage colony-stimulating factor (GM-CSF) and tumour necrosis factor alpha (TNF-α), or a combination thereof.

In one example, the ARE comprises a human antigen R or “HuR” (also known as Elav11) specific binding site. HuR is known to bind AREs increasing the stability of the mRNA.

### GC-Rich Element

As used herein, the term “GC-rich element” refers to a nucleotide sequence with a high amount of Guanine (G) and/or Cytosine (C) compared to Adenine (A) and Thymine(T)/Uracil(U). The presence of GC-rich elements in a polynucleotide (e.g. mRNA) can stabilise the mRNA.

In one example, the GC-rich element comprises a sequence of 3, or 4, or 5, or 6, or 7, or 8, or 9, or 10, or 11, or 12, or 13, or 14, or 15, or 16, or 17, or 18, or 19, or 20, or 21, or 22, or 23, or 24, or 25, or 26, or 27, or 28, or 29, or 30 nuceleotides in length.

In one example, the GC-rich element comprises between 30% and 40%, or 40% and 50%, or 50% and 60%, or 60% and 70% cytosine. For example, the GC-rich element comprises between 30% and 40% cytosine. For example, the GC-rich element comprises between 40% and 50% cytosine. For example, the GC-rich element comprises between 50% and 60% cytosine. For example, the GC-rich element comprises between 60% and 70% cytosine.

In one example, the GC-rich element comprises 30%, or 40%, or 50%, or 60%, or 70% cytosine. For example, the GC-rich element comprise 30% cytosine. For example, the GC-rich element comprises 40% cytosine. For example, the GC-rich element comprises 50% cytosine. For example, the GC-rich element comprises 60% cytosine. For example, the GC-rich element comprises 60% cytosine. For example, the GC-rich element comprises 70% cytosine.

In one example, the GC-rich element is at least 50% cytosine.

In one example, the GC-rich element is at least 60% cytosine.

In one example, the GC-rich element is at least 70% cytosine.

In one example, the GC-rich element comprises a nucleotide sequence CCCCGGCGCC. In another example, the GC-rich element comprises a nucleotide sequence CCCCGGC. In a further example, the GC-rich element comprises a nucleotide sequence GCGCCCCGCGGCGCCCCGCG.

In one example, the GC-rich element comprises a nucleotide sequence set forth in SEQ ID NO: 22 to 24. In one example, the GC-rich element comprises a nucleotide sequence set forth in SEQ ID NO: 22. In another example, the GC-rich element comprises a nucleotide sequence set forth in SEQ ID NO: 23. In a further example, the GC-rich element comprises a nucleotide sequence set forth in SEQ ID NO: 24 (CCCCGGC).

### Stem Loop

As used herein, the term “stem loop” refers to a nucleotide sequence comprising an intramolecular base pairing of two neighboured entirely or partially reverse complementary sequences to form a stem-loop. A stem-loop can occur in single-stranded DNA or, more commonly, in RNA. The stem loop can also be referred to as a hairpin or hairpin loop which usually consists of a stem and a terminal loop within a consecutive sequence, wherein the stem is formed by two neighboured entirely or partially reverse complementary sequences separated by a short sequence which builds the loop into a stem-loop structure.

The stability of the paired stem loop is determined by the length, the number of mismatched or bulges it contains, and the nucleotide composition of the paired region.

In one example, a loop of the stem loop is between 3 and 10 nucleotides in length. For example, the loop of the stem loop is between 3 and 8, or 3 and 7, or 3 and 6, or 4 and 5 nucleotides in length.

In one example, the loop of the stem loop is 4 nucleotides in length.

In one example, the stem loop is a histone stem loop. For example, the histone stem loop comprises or consist of a nucleotide sequence set for in SEQ ID NO: 25.

### 3′ Untranslated Region (3′-UTR)

In an example, the polynucleotides of the present disclosure comprise a 3′-untranslated region (3′-UTR).

As used herein, the term “3′-UTR” refers to a region of an mRNA located at the 3′ end of the translation termination codon (i.e. stop codon).

Exemplary 3′-UTRs include, for example, a 3′-UTR of arachidonate 5-lipoxygenase (ALOX5), alpha I collagen (COL1A1), tyrosine hydroxylase (TH) gene, amino-terminal enhancer of split (AES), human mitochondrial 12S rRNA (mtRNR1), a fragment and/or a variant thereof.

In one example, the 3′UTR is a 3′UTR of a Sindbis virus (SINV) or modified forms thereof. For example, the 3′UTR comprises a sequence set forth in SEQ ID NO: 27.

In one example, the 3′-UTR comprises or consists of a nucleotide sequence derived from a 3′-UTR of an albumin gene. In one example, the 3′-UTR comprises or consists of a nucleotide sequence derived from a 3′-UTR of a vertebrate α-globin gene. For example, the 3′-UTR comprises or consists of a nucleotide sequence derived from a 3′-UTR of a mammalian α-globin gene. For example, the 3′-UTR comprises or consists of a nucleotide sequence derived from a 3′-UTR of a human α-globin gene.

In one example, the 3′-UTR further comprises at least one microRNA binding site, an AU rich element (ARE), a GC-rich element, a triple helix, a stem loop, one or more stop codons or a combination thereof.

### Stop Codon

As used herein, the term “stop codon” refers to a trinucleotide sequence within a mRNA that signals the stop of protein synthesis by a ribosome.

In one example, the polynucleotide of the present disclosure comprises at least one stop codon at the 5′ end of a 3′-UTR. For example, the stop codon is selected from UAG, UAA, and UGA.

In one example, the polynucleotide comprises two consecutive stop codons comprising a sequence UGAUGA.

In one example, the polynucleotide comprises two consecutive stop codons comprising a sequence UAAUAG.

### 3′ Tailing Sequence

In an example, the polynucleotide of the present disclosure comprises one or more 3′ tailing sequences located at the 3′ end of the 3′UTR.

As described herein, the term “3′ tailing sequence” or “3′ tailing sequences” refers to a nucleotide sequence (e.g. polyadenylation signal) which induces the addition of non-encoded nucleotides to the 3′ end of a mRNA or a nucleotide sequence (e.g. poly-A sequence) located at the 3′ end of a mRNA. A skilled person will appreciate that the 3′ tailing sequence and/or products of the 3′ tailing sequence in a mRNA functions to stabilise the mRNA and/or prevent the mRNA from degradation.

As used herein, the term “interrupting linker” in reference to a poly-A or poly-C sequence refers to a single nucleotide or nucleotide sequence which are linked to, and interrupt, a stretch of consecutive adenosine or cytosine nucleotides in the poly-A or poly-C sequence. For example, the interrupting linker in a poly-A sequence is a single nucleotide or a nucleotide sequence consisting or comprising a nucleotide other than an adenosine nucleotide. For example, the interrupting linker in a poly-C sequence is a single nucleotide or a nucleotide sequence consisting or comprising a nucleotide other than a cytosine nucleotide.

In one example, the one or more 3′ tailing sequences are selected from the group consisting of a poly-A sequence, polyadenylation signal, a G-quadruplex, a poly-C sequence, a stem loop and combinations thereof.

### Poly-A Sequence

As used herein, the term “polyA sequence” refers to a nucleotide sequence of Adenine (A) located at the 3′ end of a mRNA. In the context of the present disclosure, the polyA sequence may be located within the mRNA or DNA (e.g. a DNA plasmid serving as a template for generating the mRNA by transcription of the vector).

Suitable poly-A sequence for use in the present disclosure will be apparent to the skilled person and/or are described herein. In one example, the poly-A sequence comprises consecutive (i.e. one after the other) adenosine nucleotides of any length (e.g. to 10 to 300). In one example, the poly-A sequence comprises consecutive adenosine nucleotides separated by one or more interrupting linkers. In one example, the poly-A sequence comprises consecutive adenosine nucleotides without an interrupting linker.

### Polyadenylation Signal

As used herein, the term “polyadenylation signal” refers to a nucleotide sequence which induces polyadenylation. Polyadenylation is typically understood to be the addition of a polyA sequence to a RNA (e.g. to a premature mRNA to generate a mature mRNA). The polyadenylation signal may be located within a nucleotide sequence at the 3′-end of the polynucleotide (e.g. mRNA) to be polyadenylated.

Suitable polyadenylation signals for use in the present disclosure will be apparent to the skilled person and/or described herein.

In one example, the polyadenylation signal comprises a hexamer consisting of Adenine and Uracil/Thymidine nucleotides. In one example, the hexamer sequence comprises or consists of AAUAAA.

In one example, the 3′ tailing sequence comprises a polyadenylation signal but does not comprise a polyA sequence.

### G-Quadruplex

As used herein, the term “G-quadruplex” or “G4” refers to a nucleotide sequence rich in guanine residues which forms a four stranded secondary structure. For example, the G-quadruplex is a cyclic hydrogen bonded array of four guanine nucleotides formed by G-rich sequences in both DNA and RNA.

In one example, the 3′ tailing sequence comprises a polyA sequence and a G-quadruplex. For example, the 3′ tailing sequence comprises a polyA sequence linked to a G-quadruplex to produce a polyA-G quartet.

### Poly-C Sequence

As used herein, the term “poly-C sequence” refers to a nucleotide sequence of Cytosine (C) located at the 3′ end of a mRNA. In the context of the present disclosure, the polyC sequence may be located within the mRNA or DNA (e.g. a DNA plasmid serving as a template for generating the mRNA by transcription of the vector).

Suitable poly-C sequence for use in the present disclosure will be apparent to the skilled person and/or are described herein.

In one example, the one or more 3′ tailing sequences comprises one or more poly-C sequences each comprising between 10 and 300 consecutive cytosine nucleotides. For example, the one or more poly-C sequences each comprises between 10 and 20, or 20 and 30, or 30 and 40, or 40 and 50, or 50 and 60, or 60 and 70, or 70 and 80, or 80 and 90, or 90 and 100, or 100 and 125, or 125 and 150, or 150 and 175, or 175 and 200, or 200 and 225, or 225 and 250, or 250 and 275, or 275 and 300 consecutive cytosine nucleotides. For example, the one or more poly-C sequence each comprises 10, or 20, or 30, or 40, or 50, or 60, or 70, or 80, or 90, or 100, or 125, or 150, or 175, or 200, or 225, or 250, or 275, or 300 consecutive cytosine nucleotides.

In one example, the one or more poly-C sequences is separated by an interrupting linker. For example, the fourth nucleotide sequence comprising the one or more 3′ tailing sequences comprises, in order of 5′ to 3′: consecutive cytosine nucleotides, an interrupting linker, and further consecutive cytosine nucleotides.

In one example, the interrupting linker is from 10 to 50, or 50 to 100, or 100 to 150 nucleotides in length. For example, the interrupting linker is 1, or 2, or 3, or 4, or 5, or 6, or 7, or 8, or 9, or 10, or 11, or 12, or 13, or 14, or 15, or 16, or 17, or 18, or 19, or 20, or 25, or 30, or 35, or 40, or 45, or 50, or 55, or 60, or 65, or 70, or 75, or 80, or 85, or 90, or 95, or 100, or 110, or 120, or 130, or 140, or 150 nucleotides in length.

### 5′ Cap Structure

In one example, the present disclosure provides a mRNA comprising a 5′ terminal cap structure.

As used herein, the term “5′ cap structure” refers to a structure at the 5′ terminal end of a mRNA involved in nuclear export and binds a mRNA Cap Binding Protein (CBP). The 5′ cap structure is known to stabilise mRNA through association of CBP with poly(A) binding protein to form a mature mRNA. Accordingly, the presence of a 5′ cap structure in the mRNA of the present disclosure can further increase the stability of the mRNA compared to a mRNA without the 5′ cap.

Exemplary 5′ cap structure includes, for example, anti-reverse cap analogue (ARCA), N7,2′-0-dimethyl-guanosine (mCAP), inosine, N1-methyl-guanosine, 2′fluoro-guanosine, 7-deaza-guanosine, 8-oxo-guanosine, 2-amino-guanosine, LNA-guanosine, 2-azido-guanosine, N6,2′-O-dimethyladenosine, 7-methylguanosine (m7G), Cap1, and Cap2.

Typically, an endogenous mRNA is 5′ capped with a guanosine through a (5)′-ppp-(5)′-triphosphate linkage attached to the 5′ terminal nucleotide of the mRNA. The guanosine cap can then be methylated to a 7-methylguanosine (m7G) generating a 7mG(5′)ppp(5′)N,pN2p (Cap0 structure), where N represents the first and second 5′ terminal nucleotide of the mRNA. The cap0 structure can be further 2′-O-methylated to produce 7mG(5′)ppp(5′)NlmpNp (Cap1), and/or 7mG(5′)-ppp(5′)NlmpN2mp (Cap2).

In one example, the polynucleotide of the present disclosure comprises an endogenous cap.

As used herein, the term “endogenous cap” refers to a 5′ cap synthesised in a cell. For example, endogenous cap is a natural 5′ cap or a wild-type 5′ cap. For example, the endogenous cap is a Cap0, Cap1, or Cap2 structure.

In one example, the polynucleotide of the present disclosure comprises an analog of an endogenous cap (also referred to as cap analog).

As used herein, the term “analogue thereof” in the context of an endogenous cap or “cap analog” refers to a synthetic 5′ cap. The cap analog can be used to produce 5′ capped mRNA in in vitro transcription reactions. Cap analogs may be chemically (i.e. non-ezymatically) or enzymatically synthesized and/or linked to a nucleotide (e.g. 5′ terminal nucleotide of an mRNA). Exemplary cap analogs are commercially available and include, for example, 3″-O-Me-m7G(5′)ppp(5′)G, G(5′)ppp(5′)A, G(5′)ppp(5′)G, m7G(5′)ppp(5′)A, m7G(5′)ppp(5′)G (New England BioLabs). In one example, the cap analog is N7,3′-O-dimethyl-guanosine-5′-triphosphate-5′-guanosine (i.e. anti-reverse cap analogue (ARCA)).

In one example, the 5′ cap structure is a non-hydrolyzable cap structure. The non-hydrolyzable cap structure can prevent decapping of the mRNA and increase the half-life of the mRNA.

In one example, the non-hydrolyzable cap structure comprises a modified nucleotide selected from a group consisting or a α-thio-guanosine nucleotide, α-methyl-phosphonate, seleno-phosphate, and a combination thereof. In one example, the modified nucleotide is linked to the 5′ end of the mRNA through an α-phosphorothiate linkage. Methods of linking the modified nucleotide to the 5′ end of the mRNA will be apparent to the skilled person. For example, using a Vaccinia Capping Enzyme (New England Biolabs).

### Modifications

In one example, the polynucleotide of the present disclosure comprises one or more modificiation(s). Typically, modifications are introduced into a polynucleotide (e.g. mRNA) to increase the translation efficiency and/or stability of the polynucleotide. Suitable modifications to the polynucleotide will be apparent to the skilled person and/or described herein.

In one example, the first nucleotide sequence comprising the 5′-UTR and/or the fragment thereof is modified. Modification of the first nucleotide sequences comprising the 5′-UTR and/or the fragment thereof results in a variant of the 5′-UTR and/or the fragment thereof.

In one example, one or more nucleotide sequence(s) of the polynucleotide are codon optimized. Method of codon optimization will be apparent to the skilled person and/or described herein. For example, tools for codon optimization of polynucleotide include, for example, GeneArt GeneOptimizer (Thermofisher®) or GenSmart® (GeneScript®).

In one example, the polynucleotide is modified to increase the amount of Guanine (G) and/or Cytosine (C) in the polynucleotide. The amount of G/C in the polynucleotide (i.e. G/C content) can influence the stability of the polynucleotide. Accordingly, polynucleotide comprising an increased amount of G/C nucleotides can be functionally more stable than polynucleotides containg a large amount of Adenine (A) and Thymine (T) or Uracil (U) nucleotides. The G/C content is increased by substituting A or T nucleotides with G or C nucleotides.

In one example, the G/C content is increased in the first and/or second nucleotide sequence encoding the first antigen of interest and/or adjuvant. For example, the G/C content is increased in the first and/or adjuvant sequence. The modification(s) in the first and/or second and/or one or more nucleotide sequences takes advantage of the ability of substituting codons that contain less favourable combinations of nucleotides (in terms of mRNA stability) with alternative codons encoding the same amino acid, or encoding amino acid(s) of similar chemistry (e.g. conserved amino acid substitution). For example, the G/C content is increased by substituting codons containing A or T nucleotides with codons containing G or C nucleotides that encode for the same amino acid. For example, the G/C content is increased by substituting codons containing A or T nucleotides with codons containing G or C nucleotides that encode for an amino acid of similar chemistry.

In one example, the G/C content is increased in one or more nucleotide sequences of the polynucleotide which do not encode the antigen of interest. For example, the G/C content is increased in the 5′-UTR, the fragment and/or the variant thereof. For example, the G/C content is increased in the 3′-UTR, the fragment and/or the variant thereof.

In one example, the polynucleotide comprises at least one chemically modified nucleotide.

As used herein, the term “chemical modification” or “chemical modified” in the context of a nucleotide refers to a naturally occurring nucleotides (i.e. A, T, C, G, U) which are modified by replacement, insertion or removal of individual or several atoms or atomic groups compared to the naturally occurring nucleotides. In one example, at least one naturally occurring nucleotide of the polynucleotide is replaced with a chemically modified nucleotide. In one example, at least 10%, or 20%, or 30%, or 40%, or 50%, or 60%, or 70%, or 80%, or 90%, or 100% of naturally occurring nucleotides of the polynucleotide is replaced with a chemically modified nucleotides. Suitable chemical modified nucleotides for use in the present disclosure will be apparent to the skilled person and/or described herein. Exemplary chemically modified nucleotides include, for example, N6,2′-O-dimethyl-adenosine (m6Am), 5-methyluridine (m5U), N4-acetylcytidine (ac4C), 2-thiocytidine (s2C), 2-thiouridine (s2U), 5-methylcytidine (m5C), N6-methyladenosine (m6a), pseudouridine (ψ), and 1-methylpseudouridine (m1ψ).

### Cathelicidin

Cathelicidins constitute a family of cationic antimicrobial peptides (CAPs) derived from myeloid cells. Cathelicidins, having masses ranging from 16-26 kDa, are known to be expressed in myeloid cells of humans, mice, cows, pigs, horses, sheep, rabbits and rats. They are made as precursors, in which the highly identical N-terminal preprosequences are followed by highly varied C-terminal sequences that correspond to antimicrobial peptides after removal of the prosequence at specific cleavage sites.

The cathelicidin preproregions share high intra-species indentity ranging from 75-87% for bovine and 90-97% identity for porcine preproregions. They also possess high inter-species identity ranging from 51-65%, compared to hCAP-18, thus possessing intra- and inter-species homology. Four invariant cysteins clustered in the C-terminal region of the cathelin-like propiece are arranged to form two intramolecular disulfide bonds, imposing structural constraints to the molecule.

The preproregion of cathelicidins is 128-143 amino acid residues long, including a putative 29-30 residue signal peptide and a propiece of 99-114 residues, while the C-terminal domain is 12-100 residues long. When these propeptides are secreted, they undergo limited proteolysis. In bovine and porcine neutrophils, cathelicidins are liberated by elastase-mediated cleavage, while the human cathelicidin hCAP-18 is processed extracellularly to the antimicrobial peptide LL-37 by proteinase 3. The only cathelicidin found in humans so far is the peptide LL-37 (hCAP-18/FALL-39), which is expressed in neutrophil granules and produced by bone marrow and testis, as well as other regions of the body.

The mature cathelicidins corresponding to the C-terminus are structurally diverse sequences and individual names have been given to them including bovine cathelicidins: Bacl (Bactenecinl), Bac5, Bac7, indolicidin, BMAP-27 (bovine myeloid antimicrobial peptide 27) and BMAP-28; porcine cathelicidins: PR-39 (praline-arginine-rich 39 amino-acid peptide), PMAP-36 (porcine myeloid antimicrobial peptide 36), PMAP-37, PMAP-23, protegrins, and prophenins; rabbit cathelicidins: CAP18 (cationic antimicrobial protein 18); human cathelicidins: hCAP-18/FALL-39/LL-37 (human antimicrobial protein/C-terminal derived domains are called FALL-39 or LL-37); murine cathelicidins: mCRAMP (murine cathelin-related antimicrobial peptide), MCLP (murine cathelin-like protein); rat cathelicidins: rCRAMP (rat cathelin-related antimicrobial peptide) and; sheep cathelicidins: SMAP29 (sheep myeloid antimicrobial peptide 29) and SMAP34.

Thus, in one example, the cathelicidin is selected from bovine cathelicidins: Bacl (Bactenecinl), Bac5, Bac7, indolicidin, BMAP-27 (bovine myeloid antimicrobial peptide 27) and BMAP-28; porcine cathelicidins: PR-39 (praline-arginine-rich 39 amino-acid peptide), PMAP-36 (porcine myeloid antimicrobial peptide 36), PMAP-37, PMAP-23, protegrins, and prophenins; rabbit cathelicidins: CAP18 (cationic antimicrobial protein 18); human cathelicidins: hCAP-18/FALL-39/LL-37 (human antimicrobial protein/C-terminal derived domains are called FALL-39 or LL-37); murine cathelicidins: mCRAMP (murine cathelin-related antimicrobial peptide), MCLP (murine cathelin-like protein); rat cathelicidins: rCRAMP (rat cathelin-related antimicrobial peptide) and; sheep cathelicidins: SMAP29 (sheep myeloid antimicrobial peptide 29) and SMAP34.

In one example, the cathelicidin is selected from the group consisting of dododecapeptide, indolicidin, buCATHL4A, protegrin-1, PMAP-23, BMAP-27, eCATH-2, SMAP-29, mCRAMP, rCRAMP, PMAP-36, LL-37, CAP18-FV, PMAP-37, ttLL-37, eCATH-3, Bac7 or prophenin-1 or a fragment thereof. In an example, the cathelicidin is a human cathelicidin or a fragment thereof. In another example, the cathelicidin is LL-37 or a fragment thereof.

In an example, the nucleotide sequence encoding a cathelicidin or a fragment thereof comprises a polynucleotide sequence having at least about 70%, at least about 75%, at least about 80%, at least about 85%, at least about 90% at least about 95%, at least about 96%, at least about 97%, at least about 98%, at least about 99% to any one of dododecapeptide, indolicidin, buCATHL4A, protegrin-1, PMAP-23, BMAP-27, eCATH-2, SMAP-29, mCRAMP, rCRAMP, PMAP-36, LL-37, CAP18-FV, PMAP-37, ttLL-37, eCATH-3, Bac7 or prophenin-1 or a fragment thereof.

In an example, the nucleotide sequence encoding a cathelicidin or a fragment thereof comprises a polynucleotide sequence having at least about 70%, at least about 75%, at least about 80%, at least about 85%, at least about 90% at least about 95%, at least about 96%, at least about 97%, at least about 98%, at least about 99% or 100% identity to any one of SEQ ID NOs: 1, 4, 7 or 10.

In an example, the nucleotide sequence encoding a cathelicidin or a fragment thereof comprises a polynucleotide sequence having at least about 70%, at least about 75%, at least about 80%, at least about 85%, at least about 90% at least about 95%, at least about 96%, at least about 97%, at least about 98%, at least about 99% or 100% identity to any one of SEQ ID NOs: 2, 5, 8 or 11.

In one example, the nucleotide sequence encoding a cathelicidin or a fragment thereof comprises the mRNA sequence:

(SEQ ID NO: 1)
AUGAAGACCCAGAGGGACGGCCACAGCCUGGGCAGGUGGAGCCUGGUGCU
GCUGCUGCUGGGCCUGGUGAUGCCCCUGGCCAUCAUCGCCCAGGUGCUGA
GCUACAAGGAGGCCGUGCUGAGGGCCAUCGACGGCAUCAACCAGAGGAGC
AGCGACGCCAACCUGUACAGGCUGCUGGACCUGGACCCCAGGCCCACCAU
GGACGGCGACCCCGACACCCCCAAGCCCGUGAGCUUCACCGUGAAGGAGA
CCGUGUGCCCCAGGACCACCCAGCAGAGCCCCGAGGACUGCGACUUCAAG
AAGGACGGCCUGGUGAAGAGGUGCAUGGGCACCGUGACCCUGAACCAGGC
CAGGGGCAGCUUCGACAUCAGCUGCGACAAGGACAACAAGAGGUUCGCCC
UGCUGGGCGACUUCUUCAGGAAGAGCAAGGAGAAGAUCGGCAAGGAGUUC
AAGAGGAUCGUGCAGAGGAUCAAGGACUUCCUGAGGAACCUGGUGCCCAG
GACCGAGAGC.

In one example, the nucleotide sequence encoding a cathelicidin or a fragment thereof comprises the DNA sequence:

(SEQ ID NO: 2)
ATGAAGACCCAGAGGGACGGCCACAGCCTGGGCAGGTGGAGCCTGGTGCT
GCTGCTGCTGGGCCTGGTGATGCCCCTGGCCATCATCGCCCAGGTGCTGA
GCTACAAGGAGGCCGTGCTGAGGGCCATCGACGGCATCAACCAGAGGAGC
AGCGACGCCAACCTGTACAGGCTGCTGGACCTGGACCCCAGGCCCACCAT
GGACGGCGACCCCGACACCCCCAAGCCCGTGAGCTTCACCGTGAAGGAGA
CCGTGTGCCCCAGGACCACCCAGCAGAGCCCCGAGGACTGCGACTTCAAG
AAGGACGGCCTGGTGAAGAGGTGCATGGGCACCGTGACCCTGAACCAGGC
CAGGGGCAGCTTCGACATCAGCTGCGACAAGGACAACAAGAGGTTCGCCC
TGCTGGGCGACTTCTTCAGGAAGAGCAAGGAGAAGATCGGCAAGGAGTTC
AAGAGGATCGTGCAGAGGATCAAGGACTTCCTGAGGAACCTGGTGCCCAG
GACCGAGAGC.

In one example, the nucleotide sequence encoding a cathelicidin or a fragment thereof comprises the mRNA sequence:

(SEQ ID NO: 4)
CUGCUGGGCGACUUCUUCAGGAAGAGCAAGGAGAAGAUCGGCAAGGAGUU
CAAGAGGAUCGUGCAGAGGAUCAAGGACUUCCUGAGGAACCUGGUGCCCA
GGACCGAGAGC.

In one example, the nucleotide sequence encoding a cathelicidin or a fragment thereof comprises the DNA sequence: CTGCTGGGCGACTTCTTCAGGAAGAGCAAGGAGAAGATCGGCAAG GAGTTCAAGAGGATCGTGCAGAGGATCAAGGACTTCCTGAGGAACCTGGT GCCCAGGACCGAGAGC (SEQ ID NO: 5). SEQ ID NOs: 4 and 5 represent gene sequences that encode a 37 amino acid C-terminus polypeptide of human CAP18 cleaved at position 103 and 104.

In one example, the nucleotide sequence encoding a cathelicidin or a fragment thereof comprises the mRNA sequence:

(SEQ ID NO: 7)
GGCCUGCUGAGGAAGGGCGGCGAGAAGAUCGGCGAGAAGCUGAAGAAGAU
CGGCCAGAAGAUCAAGAACUUCUUCCAGAAGCUGGUGCCCCAGCCCGAG.

In one example, the nucleotide sequence encoding a cathelicidin or a fragment thereof comprises the DNA sequence:

(SEQ ID NO: 8)
GGCCTGCTGAGGAAGGGCGGCGAGAAGATCGGCGAGAAGCTGAAGAAGAT
CGGCCAGAAGATCAAGAACTTCTTCCAGAAGCTGGTGCCCCAGCCCGAG.

SEQ ID NOs: 7 and 8 represent gene sequences that encode a 33 amino acid mouse cathelicidin homolog of human LL37.

In one example, the nucleotide sequence encoding a cathelicidin or a fragment thereof comprises the mRNA sequence:

(SEQ ID NO: 10)
AUCAGCAGGCUGGCCGGCCUGCUGAGGAAGGGCGGCGAGAAGAUCGGCGA
GAAGCUGAAGAAGAUCGGCCAGAAGAUCAAGAACUUCUUCCAGAAGCUGG
UGCCCCAGCCCGAG.

In one example, the nucleotide sequence encoding a cathelicidin or a fragment thereof comprises the DNA sequence:

(SEQ ID NO: 11)
ATCAGCAGGCTGGCCGGCCTGCTGAGGAAGGGCGGCGAGAAGATCGGCGAG
AAGCTGAAGAAGATCGGCCAGAAGATCAAGAACTTCTTCCAGAAGCTGGTG
CCCCAGCCCGAG.

SEQ ID NOs: 10 and 11 represent gene sequences that encode a 38 amino acid mouse cathelicidin homolog of human LL37.

The present disclosure also encompasses immunogenic compositions comprising a RNA of the disclsosure and a cathelicidin polypeptide or a fragment thereof.

In one example, the cathelicidin polypeptide is selected from bovine cathelicidins: Bacl (Bactenecinl), Bac5, Bac7, indolicidin, BMAP-27 (bovine myeloid antimicrobial peptide 27) and BMAP-28; porcine cathelicidins: PR-39 (praline-arginine-rich 39 amino-acid peptide), PMAP-36 (porcine myeloid antimicrobial peptide 36), PMAP-37, PMAP-23, protegrins, and prophenins; rabbit cathelicidins: CAP18 (cationic antimicrobial protein 18); human cathelicidins: hCAP-18/FALL-39/LL-37 (human antimicrobial protein/C-terminal derived domains are called FALL-39 or LL-37); murine cathelicidins: mCRAMP (murine cathelin-related antimicrobial peptide), MCLP (murine cathelin-like protein); rat cathelicidins: rCRAMP (rat cathelin-related antimicrobial peptide) and; sheep cathelicidins: SMAP29 (sheep myeloid antimicrobial peptide 29) and SMAP34.

In one example, the cathelicidin polypeptide is selected from the group consisting of dododecapeptide, indolicidin, buCATHL4A, protegrin-1, PMAP-23, BMAP-27, eCATH-2, SMAP-29, mCRAMP, rCRAMP, PMAP-36, LL-37, CAP18-FV, PMAP-37, ttLL-37, eCATH-3, Bac7 or prophenin-1 or a fragment thereof. In an example, the cathelicidin is a human cathelicidin or a fragment thereof. In another example, the cathelicidin is LL-37 or a fragment thereof.

In an example, the cathelicidin polypeptide comprises an amino acid sequence having at least about 70%, at least about 75%, at least about 80%, at least about 85%, at least about 90% at least about 95%, at least about 96%, at least about 97%, at least about 98%, at least about 99% to any one of dododecapeptide, indolicidin, buCATHL4A, protegrin-1, PMAP-23, BMAP-27, eCATH-2, SMAP-29, mCRAMP, rCRAMP, PMAP-36, LL-37, CAP18-FV, PMAP-37, ttLL-37, eCATH-3, Bac7 or prophenin-1 or a fragment thereof.

In an example, the cathelicidin polypeptide comprises an amino acid sequence sequence having at least about 70%, at least about 75%, at least about 80%, at least about 85%, at least about 90% at least about 95%, at least about 96%, at least about 97%, at least about 98%, at least about 99% or 100% identity to any one of SEQ ID NOs: 3, 6, 9 or 12.

In one example, the cathelicidin polypeptide comprises an amino acid sequence according to:

(SEQ ID NO: 3)
MKTQRDGHSLGRWSLVLLLLGLVMPLAIIAQVLSYKEAVLRAIDGINQRS
SDANLYRLLDLDPRPTMDGDPDTPKPVSFTVKETVCPRTTQQSPEDCDFK
KDGLVKRCMGTVTLNQARGSFDISCDKDNKRFALLGDFFRKSKEKIGKEF
KRIVQRIKDFLRNLVPRTES.

In one example, the cathelicidin polypeptide comprises an amino acid sequence according to:

(SEQ ID NO: 6)
LLGDFFRKSKEKIGKEFKRIVQRIKDFLRNLVPRTES.

This sequence is a 37 amino acid C-terminus polypeptide of human CAP18 cleaved at position 103 and 104.

In one example, the cathelicidin polypeptide comprises an amino acid sequence according to:

GLLRKGGEKIGEKLKKIGQKIKNFFQKLVPQPE (SEQ ID NO: 9). This sequence is a 33 amino acid mouse cathelicidin homolog of human LL37.

In one example, the cathelicidin polypeptide comprises an amino acid sequence according to: ISRLAGLLRKGGEKIGEKLKKIGQKIKNFFQKLVPQPE (SEQ ID NO: 12). This is a 38 amino acid sequence mouse homolog of human LL37.

In another example, a suitable control cathelicidin polypeptide comprises an amino acid sequence according to:

MGLCPGYKIPQKILDSLDNSFKLFANLLFRFPEEITQQ (SEQ ID NO: 13). This is the reverse orientation of the human LL37 sequence.

The present disclosure also encompasses fragments of a cathelicidin polypeptide as well as cathelicidin polypeptides with one or more mutations such as substitution(s), deletion(s) and/or addition(s). Preferably not more than 10% of the amino acids of a given cathelicidin polypeptide, or fragment thereof, shall be substituted, deleted or added. Such mutations are performed according to standard knowledge, e.g. hydrophobic amino acid residues are exchanged by other hydrophobic residues, etc. Such mutants and/or fragments will be understood to retain a similar or substantially the same level of function as the wild type or non-mutated or full length counterpart. For example, the effect of a cathelicidin fragment or mutant on immunogenicity will be similar or substantially the same when compared to the wild type, non-mutated or full length counterpart.

### Antigens

The polynucleotide of the present disclosure comprises a first nucleotide sequence encoding an antigen of interest operably linked to a promoter, and a second nucleotide sequence encoding a cathelicidin or a fragment thereof. For example, the antigen of interest is an antigen polypeptide, a fragment and/or a variant thereof which can induce an immune response in the subject.

The cRNA of the present disclosure comprises a first nucleotide sequence encoding an antigen of interest operably linked to a promoter, and a second nucleotide sequence encoding a cathelicidin or a fragment thereof. For example, the antigen of interest is an antigen polypeptide, a fragment and/or a variant thereof which can induce an immune response in the subject.

The self-replicating RNA of the present disclosure comprises a first nucleotide sequence encoding an antigen of interest operably linked to a promoter, and a second nucleotide sequence encoding a cathelicidin or a fragment thereof. For example, the antigen of interest is an antigen polypeptide, a fragment and/or a variant thereof which can induce an immune response in the subject.

An antigenic polypeptide, a fragment and/or the variant thereof suitable for use in the polynucleotide described herein will be apparent to the skilled person and, for example, include proteins and peptides derived from any pathogen. For example, the antigen is a virus, bacteria, a fungus, or a protozoan.

Antigens suitable for use in accordance with the present disclosure will be apparent to the skilled person and, for example, include proteins and peptides derived from any pathogen. For example, the antigen is a virus, bacteria, a fungus or a protozoan.

### Viral Antigens

In one example, the antigen is a viral antigen.

Suitable viral antigens will be apparent to the skilled person and include, for example, proteins and peptides from a Orthomyxoviruses (e.g., Influenza A, B and C), Paramyxoviridae viruses (Pneumoviruses (e.g., Respiratory syncytial virus (RSV), Bovine respiratory syncytial virus, Pneumonia virus of mice, and Turkey rhinotracheitis virus), Paramyxovirus types 1-4 (PIV), Mumps, Sendai viruses, Simian virus 5)), Bovine parainfluenza virus, Nipahvirus, Henipavirus and Newcastle disease virus), Poxviridae (e.g., Variola vera, including but not limited to, Variola major and Variola minor, Metapneumoviruses, such as human metapneumovirus (hMPV) and avian metapneumoviruses (aMPV)), Morbilliviruses (e.g., Measles), Picornaviruses (e.g., Enteroviruses, Rhinoviruses, Heparnavirus, Parechovirus, Cardioviruses and Aphthoviruses), Enteroviruseses (e.g., Poliovirus types 1, 2 or 3, Coxsackie A virus types 1 to 22 and 24, Coxsackie B virus types 1 to 6, Echovirus (ECHO) virus types 1 to 9, 11 to 27 and 29 to 34 and Enterovirus 68 to 71), Bunyaviruses (e.g., California encephalitis virus), Phlebovirus (e.g., Rift Valley Fever virus), Nairovirus (e.g., Crimean-Congo hemorrhagic fever virus), Heparnaviruses (e.g., Hepatitis A virus (HAV)), Togaviruses (e.g., Rubivirus, an Alphavirus, or an Arterivirus), Flaviviruses (e.g., Tick-borne encephalitis (TBE) virus, Dengue (types 1, 2, 3 or 4) virus, Yellow Fever virus, Japanese encephalitis virus, Kyasanur Forest Virus, West Nile encephalitis virus, St. Louis encephalitis virus, Russian spring-summer encephalitis virus, Powassan encephalitis virus), Pestiviruses (e.g., Bovine viral diarrhea (BVDV), Classical swine fever (CSFV) or Border disease (BDV)), Hepadnaviruses (e.g., Hepatitis B virus, Hepatitis C virus), Rhabdoviruses (e.g., Lyssavirus (Rabies virus) and Vesiculovirus (VSV)), Caliciviridae (e.g., Norwalk virus, and Norwalk-like Viruses (e.g., Hawaii Virus and Snow Mountain Virus); Coronaviruses (e.g., severe acute respiratory syndrome (SARS) coronavirus (SARS-CoV), SARS coronavirus 2 (SARS-CoV-2), Middle East respiratory syndrome (MERS) coronavirus (MERS-CoV), Avian infectious bronchitis (IBV), Mouse hepatitis virus (MHV), and Porcine transmissible gastroenteritis virus (TGEV)), Retroviruses (e.g., Oncovirus, a Lentivirus or a Spumavirus), Reoviruses (e.g., Orthoreo virus, a Rotavirus, an Orbivirus, or a Coltivirus), Parvoviruses (e.g., Parvovirus B 19), Delta hepatitis virus (HDV), Hepatitis E virus (HEV), Human Herpesviruses (e.g., Herpes Simplex Viruses (HSV), Varicella-zoster virus (VZV), Epstein-Barr virus (EBV), Cytomegalovirus (CMV), Human Herpesvirus 6 (HHV6), Human Herpesvirus 7 (HHV7), and Human Herpesvirus 8 (HHV8)), Papovaviruses (e.g., Papillomaviruses and Polyomaviruses), Adenoviruess and Arenaviruses.

In one example, the antigen is a viral antigen from a respiratory virus. Respiratory viral antigens that can be encoded by the self-replicating RNA will be apparent to the skilled person and include, for example, proteins and peptides from a Orthomyxoviruses (e.g., Influenza A, B and C), Paramyxoviridae viruses (Pneumoviruses (e.g., Respiratory syncytial virus (RSV), Bovine respiratory syncytial virus, Pneumonia virus of mice, and Turkey rhinotracheitis virus), Paramyxoviruses (PIV), and Metapneumovirus such as human metapneumovirus (hMPV) and avian metapneumoviruses (aMPV)), Picornaviruses (e.g., Rhinoviruses) and Coronaviruses (e.g., severe acute respiratory syndrome (SARS) coronavirus (SARS-CoV), SARS coronavirus 2 (SARS-CoV-2), Middle East respiratory syndrome (MERS) coronavirus (MERS-CoV), Avian infectious bronchitis (IBV), Mouse hepatitis virus (MHV)).

In one example, the antigen is a viral antigen from an influenza virus.

In another example, the antigen is a viral antigen from a coronavirus.

### Bacterial Antigens

In one example, the antigen is a bacterial antigen.

Suitable bacterial antigens will be apparent to the skilled person and include, for example, proteins and peptides from a Neisseria meningitides, Streptococcus pneumoniae, Streptococcus pyogenes, Moraxella catarrhalis, Bordetella pertussis, Burkholderia sp. (e.g., Burkholderia mallei, Burkholderia pseudomallei and Burkholderia cepacia), Staphylococcus aureus, Haemophilus influenzae, Clostridium tetani (Tetanus), Clostridium perfringens, Clostridium botulinums, Cornynebacterium diphtheriae (Diphtheria), Pseudomonas aeruginosa, Legionella pneumophila, Coxiella burnetii, Brucella sp. (e.g., B. abortus, B. canis, B. melitensis, B. neotomae, B. ovis, B. suis and B. pinnipediae), Francisella sp. (e.g., F. novicida, F. philomiragia and F. tularensis), Streptococcus agalactiae, Neiserria gonorrhoeae, Chlamydia trachomatis, Treponema pallidum (Syphilis), Haemophilus ducreyi, Enterococcus faecalis, Enterococcus faecium, Helicobacter pylori, Staphylococcus saprophyticus, Yersinia enterocolitica, E. coli, Bacillus anthracis (anthrax), Yersinia pestis (plague), Mycobacterium tuberculosis, Rickettsia, Listeria, Chlamydia pneumoniae, Vibrio cholerae, Salmonella typhi (typhoid fever), Borrelia burgdorferi, Porphyromonas sp, Klebsiella sp.

### Fungal Antigens

In one example, the antigen is a fungal antigen.

Suitable fungal antigens will be apparent to the skilled person and include, for example, proteins and peptides from Dermatophytes (including Epidermophyton floccusum, Microsporum audouini, Microsporum canis, Microsporum distortum, Microsporum equinum, Microsporum gypsum, Microsporum nanum, Trichophyton concentricum, Trichophyton equinum, Trichophyton gallinae, Trichophyton gypseum, Trichophyton megnini, Trichophyton mentagrophytes, Trichophyton quinckeanum, Trichophyton rubrum, Trichophyton schoenleini, Trichophyton tonsurans, Trichophyton verrucosum, T verrucosum var. album, var. discoides, var. ochraceum, Trichophyton violaceum, and/or Trichophyton faviforme), Aspergillus fumigatus, Aspergillus flavus, Aspergillus niger, Aspergillus nidulans, Aspergillus terreus, Aspergillus sydowii, Aspergillus flavatus, Aspergillus glaucus, Blastoschizomyces capitatus, Candida albicans, Candida enolase, Candida tropicalis, Candida glabrata, Candida krusei, Candida parapsilosis, Candida stellatoidea, Candida kusei, Candida parakwsei, Candida lusitaniae, Candida pseudotropicalis, Candida guilliermondi, Cladosporium carrionii, Coccidioides immitis, Blastomyces dermatidis, Cryptococcus neoformans, Geotrichum clavatum, Histoplasma capsulatum, Klebsiella pneumoniae, Microsporidia, Encephalitozoon spp., Septata intestinalis and Enterocytozoon bieneusi.

### Protazoan Antigens

In one example, the antigen is a protazoan antigen.

Suitable protazoan antigens will be apparent to the skilled person and include, for example, proteins and peptides from Entamoeba histolytica, Giardia lambli, Cryptosporidium parvum, Cyclospora cayatanensis and Toxoplasma.

### Methods of Production

Suitable methods for the production of a polynucleotide, a cRNA and/or a self-replicating RNA of the present disclosure will be apparent to the skilled person and/or described herein.

In one example, the polynucleotide is DNA. For example, the polynucleotide is a plasmid DNA.

In one example, the cRNA is produced using a plasmid DNA. In one example, the self-replicating RNA is produced using a plasmid DNA. The skilled person will understand that plasmid DNA is relatively stable. Briefly, competent bacterial cells (e.g., Escherichia coli) cells are transformed with a DNA plasmid encoding a self-replicating RNA of the present disclosure. Individual bacterial colonies are isolated and the resultant plasmid DNA amplified in E. coli cultures.

In one example, the plasmid DNA is isolated following fermentation. For example, the plasmid DNA is isolated using a commercially available kit (e.g., Maxiprep DNA kit), or other routine methods known to the skilled person. Following isolation, plasmid DNA is linearized by restriction digest (i.e., using a restricting enzyme). Restriction enzymes are removed using methods known in the art, including for example phenol/chloroform extraction and ethanol precipitation.

In one example, mRNA is made by in vitro transcription from a linearized DNA template using a RNA polymerase (e.g., T7 RNA polymerase). Following in vitro transcription, the DNA template is removed by DNase digestion. The skilled person will understand that synthetic mRNA capping is performed to correct mRNA processing and contribute to stabilization of the mRNA. In one example, the mRNA is enzymatically 5′-capped. For example, the 5′ cap is a cap0 structure or a cap1 structure. In one example, the 5′ cap is a cap0 structure, for example, the 5′-cap (i.e., cap0) consists of an inverted 7-methylguanosine connected to the rest of the mRNA via a 5′-5′ triphosphate bridge. In one example, the 5′ cap is a cap1 structure, for example, the 5′-cap (i.e., cap1) consists of the cap0 with an additional methylation of the 2′ O position of the initiating nucleotide.

In one example, the mRNA is purified. Various methods for purifying mRNA will be apparent to the skilled person. For example, the mRNA is purified using lithium chloride (LiCl) precipitation. In another example, the mRNA is purified using tangential flow filtration (TFF). Following purification, the mRNA is resuspended in e.g., nuclease-free water.

### Compositions

The present disclosure provides an immunogenic composition comprising a polynucleotide of the present disclosure.

The present disclosure also provides an immunogenic composition comprising a cRNA of the present disclosure.

The present disclosure further provides an immunogenic composition comprising a self-replicating RNA of the present disclosure. In particular embodiments, the present disclosure also provides an immunogenic composition comprising a self-replicating RNA of the present disclosure and a cathelicidin polypeptide or a fragment thereof.

The present disclosure also provides a pharmaceutical composition comprising an immunogenic composition of the present disclosure and a pharmaceutically acceptable carrier.

It will be apparent to the skilled person and/or described herein, that the polynucleotide, cRNA and/or self-replicating RNA of the present disclosure may be present as naked RNA or in combination with lipids, polymers or other delivery system that facilitates entry into the cells.

### Delivery Systems

In one example, the pharmaceutical composition of the present disclosure further comprises a LNP, a polymeric microparticle and an oil-in-water emulsion. For example, the polynucleotide, the cRNA and/or the self-replicating RNA is encapsulated in, bound to or adsorbed on a LNP, a polymeric microparticle, or an oil-in-water emulsion.

### Lipid Nanoparticles

In one example, the present disclosure provides a pharmaceutical composition comprising a polynucleotide comprising a first nucleotide sequence encoding an antigen operably linked to a regulatory element, and a second nucleotide sequence encoding a cathelicidin operably linked to a regulatory element, wherein the composition further comprises a lipid nanoparticle (LNP).

It will be apparent that the term “lipid nanoparticle” or “LNP” refers to any lipid composition, including, but not limited to, liposomes or vesicles, where an aqueous volume is encapsulated by amphipathic lipid bilayers (e.g., single; unilamellar or multiple; multilamellar) micelle-like lipid nanoparticles having a non-aqueous core and solid lipid nanoparticles, wherein solid lipid nanoparticles lack lipid bilayers.

Lipid nanoparticles suitable for use in the present disclosure will be apparent to the skilled person and/or are described herein. The lipids can have an anionic, cationic or zwitterionic hydrophilic head group.

In one example, the lipid nanoparticle comprises a PEG-lipid, a sterol structural lipid and/or a neutral lipid. In one example, the lipid nanoparticle further comprises a cationic lipid. In one example, the lipid nanoparticle does not comprise a cationic lipid.

In one example, the LNP comprises a PEG-lipid. For example, the PEG-lipid is selected from the group consisting of PEG-c-DMG, PEG-DMG, PEG-DLPE, PEG-DMPE, PEG-DPPC, a PEG-DSPE lipid and combinations thereof.

In one example, the LNP comprises a structural lipid. For example, the structural lipid is selected from the group consisting of cholesterol fecosterol, sitosterol, campesterol, stigmasterol, brassicasterol, ergosterol, tomatidine, tomatine, ursolic acid and alpha-tocopherol and combinations thereof.

In one example, the LNP comprises a neutral lipid. Exemplary phospholipids (anionic or zwitterionic) for use in the present disclosure include, for example, phosphatidylethanolamines, phosphatidylcholines, phosphatidylserines, and phosphatidylglycerols. For example, the neutral lipid is selected from the group consisting of 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine (DSPE), 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), and sphingomyelin and combinations thereof.

In one example, the LNP comprises a cationic lipid. Exemplary cationic lipids include, but are not limited to, dioleoyl trimethylammonium propane (DOTAP), 1,2-distearyloxy-N,N-dimethyl-3-aminopropane (DSDMA), 1,2-dioleyloxy-N,Ndimethyl-3-aminopropane (DODMA), 1,2-dilinoleyloxy-N,N-dimethyl-3-aminopropane (DLinDMA), 1,2-dilinolenyloxy-N,N-dimethyl-3-aminopropane (DLenDMA), 2,5-bis((9z,12z)-octadeca-9,12,dien-1-yloxyl)benzyl-4-(dimethylamino)butnoate (LKY750). In one example, the phospholipid is 2,5-bis((9z,12z)-octadeca-9,12,dien-1-yloxyl)benzyl-4-(dimethylamino)butnoate (LKY750). Exemplary zwitterionic lipids include, but are not limited to, acyl zwitterionic lipids and ether zwitterionic lipids, such as dipalmitoylphosphatidylcholine (DPPC), dioleoylphosphatidylcholine (DOPC) and dodecylphosphocholine. The lipids can be saturated or unsaturated.

### Polymeric Microparticles

In one example, the pharmaceutical composition of the present disclosure further comprises a polymeric microparticle.

The skilled person will be aware that various polymers can form microparticles to encapsulate or adsorb the polynucleotide, the cRNA and/or the self-replicating RNA of the present disclosure. It will be apparent that use of a substantially non-toxic polymer means that particles are safe, and the use of a biodegradable polymer means that the particles can be metabolised after delivery to avoid long-term persistence. Useful polymers are also sterilisable, to assist in the preparation of pharmaceutical grade formulations.

Exemplary non-toxic and biodegradable polymers include, but are not limited to, poly(α-hydroxy acids), polyhydroxy butyric acids, polylactones (including polycaprolactones), polydioxanones, polyvalerolactone, polyorthoesters, polyanhydrides, polycyanoacrylates, tyrosine-derived polycarbonates, polyvinyl-pyrrolidinones or polyester-amides, and combinations thereof.

### Oil-In-Water Cationic Emulsions

In one example, the pharmaceutical composition of the present disclosure further comprises an oil-in-water cationic emulsion.

Suitable oils for use in an oil-in-water emulsion will be apparent to the skilled person and/or are described herein. For example, the emulsion comprises one or more oils derived, for example, from an animal (e.g., fish) or a vegetable source (e.g., nuts, seeds, grains). The skilled person will recognise that biocompatible and biodegradable oils are preferentially used. Exemplary animal oils (i.e., fish oils) include cod liver oil, shark liver oils, and whale oil. Exemplary vegetable oils include peanut oil, coconut oil, olive oil, soybean oil, jojoba oil, safflower oil, cottonseed oil, sunflower seed oil, sesame seed oil, corn oil.

In addition to the oil, the oil-in-water emulsion also comprises a cationic lipid to facilitate formation and stabilisation of the emulsion. Suitable cationic lipids will be apparent to the skilled person and/or are described herein. Exemplary cationic lipids include, but are not limited to, limited to: 1,2-dioleoyloxy-3-(trimethylammonio)propane (DOTAP), 3′-[N—(N′,N′-Dimethylaminoethane)-carbamoyl] Cholesterol (DC Cholesterol), dimethyldioctadecyl-ammonium (DDA), 1,2-Dimyristoyl-3-Trimethyl-AmmoniumPropane (DMTAP), dipalmitoyl[C16:0]trimethyl ammonium propane (DPTAP) and distearoyltrimethylammonium propane (DSTAP).

In some examples, the oil-in-water emulsion also comprises a non-ionic surfactant and/or a zwitterionic surfactant. The skilled person will be aware of surfactants suitable for use in the present disclosure. Exemplary surfactants include, but are not limited to: the polyoxyethylene sorbitan esters surfactants (e.g., polysorbate 20 and polysorbate 80) and copolymers of ethylene oxide (EO), propylene oxide (PO), and/or butylene oxide (BO).

### Pharmaceutically Acceptable Carrier

Suitably, in compositions or methods for administration of the cRNA and/or the self-replicating RNA of the disclosure to a subject, the cRNA and/or the self-replicating RNA is combined with a pharmaceutically acceptable carrier as is understood in the art. Accordingly, one example of the present disclosure provides a composition (e.g., a pharmaceutical composition) comprising the self-replicating RNA of the disclosure (and any delivery system) combined with a pharmaceutically acceptable carrier. Another example of the present disclosure provides a composition (e.g., a pharmaceutical composition) comprising the cRNA of the disclosure (and any delivery system) combined with a pharmaceutically acceptable carrier.

In general terms, by “carrier” is meant a solid or liquid filler, binder, diluent, encapsulating substance, emulsifier, wetting agent, solvent, suspending agent, coating or lubricant that may be safely administered to any subject, e.g., a human. Depending upon the particular route of administration, a variety of acceptable carriers, known in the art may be used, as for example described in Remington's Pharmaceutical Sciences (Mack Publishing Co. N.J. USA, 1991).

The cRNA and/or the self-replicating RNA of the present disclosure is useful for parenteral, topical, oral, or local administration, intramuscular administration, aerosol administration, or transdermal administration, for prophylactic or for therapeutic treatment. In one example, the self-replicating RNA is administered parenterally, such as intramuscularly, subcutaneously or intravenously. For example, the self-replicating RNA is administered intramuscularly. In another example, the cRNA is administered parenterally, such as intramuscularly, subcutaneously or intravenously. For example, the cRNA is administered intramuscularly.

Formulation of a cRNA and/or a self-replicating RNA to be administered will vary according to the route of administration and formulation (e.g., solution, emulsion, capsule) selected. An appropriate pharmaceutical composition comprising a cRNA and/or a self-replicating RNA to be administered can be prepared in a physiologically acceptable carrier. For solutions or emulsions, suitable carriers include, for example, aqueous or alcoholic/aqueous solutions, emulsions or suspensions, including saline and buffered media. Parenteral vehicles can include sodium chloride solution, Ringer's dextrose, dextrose and sodium chloride, lactated Ringer's or fixed oils. A variety of appropriate aqueous carriers are known to the skilled artisan, including water, buffered water, buffered saline, polyols (e.g., glycerol, propylene glycol, liquid polyethylene glycol), dextrose solution and glycine. Intravenous vehicles can include various additives, preservatives, or fluid, nutrient or electrolyte replenishers (See, generally, Remington's Pharmaceutical Science, 16th Edition, Mack, Ed. 1980). The compositions can optionally contain pharmaceutically acceptable auxiliary substances as required to approximate physiological conditions such as pH adjusting and buffering agents and toxicity adjusting agents, for example, sodium acetate, sodium chloride, potassium chloride, calcium chloride and sodium lactate. The cRNA and/or self-replicating RNA can be stored in the liquid stage or can be lyophilized for storage and reconstituted in a suitable carrier prior to use according to art-known lyophilization and reconstitution techniques.

The optimum concentration of the active ingredient(s) in the chosen medium can be determined empirically, according to procedures known to the skilled artisan, and will depend on the ultimate pharmaceutical formulation desired.

Upon formulation, compositions of the present disclosure will be administered in a manner compatible with the dosage formulation and in such amount as is therapeutically/prophylactically effective. The dosage ranges for the administration of the cRNA and/or self-replicatng RNA of the disclosure are those large enough to produce the desired effect. For example, the composition comprises an effective amount of the self-replicating RNA. In one example, the composition comprises a therapeutically effective amount of the self-replicating RNA. In another example, the composition comprises a prophylactically effective amount of the self-replicating RNA. In one example, the composition comprises an effective amount of the cRNA. In one example, the composition comprises a therapeutically effective amount of the cRNA. In another example, the composition comprises a prophylactically effective amount of the cRNA.

The dosage should not be so large as to cause adverse side effects. Generally, the dosage will vary with the age, condition, sex and extent of the disease in the patient and can be determined by one of skill in the art. The dosage can be adjusted by the individual physician in the event of any complication.

Dosage can vary from about 0.1 mg/kg to about 300 mg/kg, e.g., from about 0.2 mg/kg to about 200 mg/kg, such as, from about 0.5 mg/kg to about 20 mg/kg, in one or more dose administrations daily, for one or several days.

In some examples, the cRNA and/or the self-replicating RNA is administered at an initial (or loading) dose which is higher than subsequent (maintenance doses). For example, the cRNA and/or the self-replicating RNA is administered at an initial dose of between about 10 mg/kg to about 30 mg/kg. The cRNA and/or the self-replicating RNA is then administered at a maintenance dose of between about 0.0001 mg/kg to about 10 mg/kg. The maintenance doses may be administered every 7-35 days, such as, every 7 or 14 or 28 days.

In some examples, a dose escalation regime is used, in which the cRNA and/or the self-replicating RNA is initially administered at a lower dose than used in subsequent doses. This dosage regime is useful in the case of subject's initially suffering adverse events In the case of a subject that is not adequately responding to treatment, multiple doses in a week may be administered. Alternatively, or in addition, increasing doses may be administered.

A subject may be retreated with the cRNA and/or the self-replicating RNA of the present disclosure. A subject may be retreated with the cRNA and/or the self-replicating RNA, by being given more than one exposure or set of doses, such as at least about two exposures of the binding protein, for example, from about 2 to 60 exposures, and more particularly about 2 to 40 exposures, most particularly, about 2 to 20 exposures.

In one example, any retreatment may be given when signs or symptoms of disease return.

In another example, any retreatment may be given at defined intervals. For example, subsequent exposures may be administered at various intervals, such as, for example, about 24-28 weeks or 48-56 weeks or longer. For example, such exposures are administered at intervals each of about 24-26 weeks or about 38-42 weeks, or about 50-54 weeks.

In the case of a subject that is not adequately responding to treatment, multiple doses in a week may be administered. Alternatively, or in addition, increasing doses may be administered.

In another example, for subjects experiencing an adverse reaction, the initial (or loading) dose may be split over numerous days in one week or over numerous consecutive days.

Administration of the cRNA and/or the self-replicating RNA according to the methods of the present disclosure can be continuous or intermittent, depending, for example, on the recipient's physiological condition, whether the purpose of the administration is therapeutic or prophylactic, and other factors known to skilled practitioners. The administration of the cRNA and/or the self-replicating RNA may be essentially continuous over a preselected period of time or may be in a series of spaced doses, e.g., either during or after development of a condition.

### Screening Assays

Suitable methods for selecting a cRNA and/or a self-replicating RNA of the present disclosure are available to those skilled in the art. Assays may be conducted to assess the efficiency and efficacy of the RNA including, for example, serology and immune responses.

Antigen and/or Adjuvant Expression

In one example, the self-replicating RNA is assessed for expression of the polynucleotide/s of interest. In another example, the cRNA is assessed for expression of the polynucleotide/s of interest.

For example, antigen expression is detected using antibodies against the polynucleotide/s of interest. In one example, expression of the adjuvant i.e., cathelicidin can be detected using commercially available antibodies including ABIN7232683, suitable for FACS analysis and PA5-120383, suitable for Western blotting.

In one example, the number of cells positive for antigen expression is measured by e.g., fluorescence-activated cell sorting (FACS). In another example the mean fluorescence intensity (MFI) is determined using e.g., FACS. In a further example, the specific potency value or the probability of successful transfection per unit mass of RNA is calculated.

### Microneutralization Assay

In one example, the self-replicating RNA (naked and/or formulated) is assessed for antibody responses. In one example, the cRNA (naked and/or formulated) is assessed for antibody responses. For example, the cRNA and/or the self-replicating RNA is assessed using a microneutralisation assay. Methods of performing a microneutralization assay will be apparent to the skilled person. In one example, the microneutralization assay is a short form assay. For one example, a virus fluorescent focus-based microneutralization assay is performed. In another example, the microneutralization assay is a long form assay.

### Hemagglutination Inhibition (HAI) Assay

In one example, the self-replicating RNA (naked and/or formulated) is assessed for antibody responses. In one example, the cRNA (naked and/or formulated) is assessed for antibody responses. For example, the cRNA and/or self-replicating RNA is assessed using a hemagglutination inhibition (HAI) assay. Methods of performing a HAI assay will be apparent to the skilled person and/or described, for example, in WHO (2011) Manual for the laboratory diagnosis and virological surveillance of influenza: WHO Press, World Health Organization.

### Antigen Specific T Cell Responses

In one example, the self-replicating RNA is assessed for its ability to induce antigen specific T cell responses. In one example, the cRNA is assessed for its ability to induce antigen specific T cell responses. Methods of assessing induction of antigen specific T cell responses will be apparent to the skilled person and/or are described herein.

For example, antigen-specific T cell detection is performed on splenic cultures. Briefly, splenocyte cultures are established in T cell medium and cell cultures are either stimulated with antigenic peptides or unstimulated. In one example, antigen-specific T cell responses are determined using flow cytometry.

### Neutralising Assays

The self-replicating RNA of the disclosure may be screened in vitro for their ability to bind to a polypeptide of interest (i.e., antigen) and neutralise binding. Suitable assays will be apparent to the skilled person and include, for example, a Vero microneutralisation assay, a sVNT assay, or a psuedovirus neutralisation assay (using e.g., HEK-293T cells or HeLa-ACE2 cells).

In one example, the neutralization assay is a Vero microneutralization assay. Briefly, wild-type virus is passaged in Vero cells (i.e., the Vero lineage isolated from kidney epithelial cells extracted from an African green monkey). Serial two-fold dilutions of a test protein are incubated with 100 TCID₅₀ (i.e., median tissue culture infectious dose) of virus for 1 hour and residual virus infectivity is assessed in Vero cells; viral cytopathic effect is read, for example, on day 5. The neutralising antibody titre is calculated using the Reed/Muench method as previously described (Houser et al., 2016; Subbarao et al 2004).

In one example, the neutralization assay is a surrogate neutralization test (sVNT). Briefly, the wells of a plate are coated with a relevant receptor protein in carbonate-bicarbonate coating buffer (e.g., pH 9.6). HRP-conjugated virus and HRP-conjugated virus pre-incubated with test proteins is added to the receptor at different concentrations and incubated, for example, for 1 h at room temperature. Unbound HRP conjugated antigens are removed by washing. Colorimetric signal is developed on the enzymatic reaction of HRP with chromogenic substrate, e.g., 3,3′,5,5′-tetramethylbenzidine (TMB). In one example, the absorbance reading at 450 nm and 570 nm is acquired.

In one example, the neutralisation is a psuedovirus neutralisation assay. Briefly, HIV reporter virus pseudotyped with an antigen is produced by co-transfection of plasmids comprising the antigen together with a viral backbone plasmid (e.g., pDR-NL Aenv FLUC) into e.g., HEK-293T cells. Pseudovirus is harvested post transfection and clarified by filtration. Virus stock titres, reported as Relative Luciferase Units infectious dose (RLU), are calculated by limiting dilution infections in Hela-hACE2 cells measuring luciferase activity as a read-out for viral infection.

### Methods of Treatment or Prevention

The present disclosure provides, for example, methods of treating or preventing or delaying progression of a disease or disorder in a subject in need thereof. In some examples of the present disclosure the subject has a disease or condition but has not been clinically diagnosed as having the disease or condition. Thus in an example, the subject may exhibit one or more symptoms of the disease or condition but the disease or condition is not yet clinically detectable.

A subject in need may be an individual who is displaying a symptom of a disease or disorder or who has been diagnosed with a disease or disorder. Further, a subject in need thereof may be one who has been clinically or biochemically determined to be infected with a disease or disorder. In one embodiment, the subject may be asymptomatic.

A reduction in a disease or disorder may be determined using any method known in the art or described herein. For example, where the disease or disorder is a viral infection, the determination comprises measuring viral load in a sample from the subject after treatment and comparing it to viral load in a sample from the same subject before treatment. In an example, the sample is taken from the respiratory tract, e.g., the upper respiratory tract, for example the nose or pharynx (i.e. throat). Alternatively, responsiveness to a treatment may result in lessening of the severity of one or more of the symtpoms described herein.

### Kits

Another example of the disclosure provides kits containing a self-replicating RNA of the present disclosure useful for the treatment or prevention of a disease or disorder as described above.

Another example of the disclosure provides kits containing a cRNA of the present disclosure useful for the treatment or prevention of a disease or disorder as described above.

In one example, the kit comprises (a) a container comprising a self-replicating RNA optionally in a delivery system and/or a pharmaceutically acceptable carrier or diluent; and (b) a package insert with instructions for treating or preventing a disease or disorder in a subject.

In one example, the kit comprises (a) a container comprising a RNA optionally in a delivery system and/or a pharmaceutically acceptable carrier or diluent; and (b) a package insert with instructions for treating or preventing a disease or disorder in a subject.

In accordance with this example of the disclosure, the package insert is on or associated with the container. Suitable containers include, for example, bottles, vials, syringes, etc. The containers may be formed from a variety of materials such as glass or plastic. The container holds or contains a composition that is effective for a disease or disorder of the disclosure and may have a sterile access port (for example, the container may be an intravenous solution bag or a vial having a stopper pierceable by a hypodermic injection needle). At least one active agent in the composition is the self-replicating RNA and/or the cRNA. The label or package insert indicates that the composition is used for treating a subject eligible for treatment with specific guidance regarding dosing amounts and intervals of treatment and any other medicament being provided. The kit may further comprise an additional container comprising a pharmaceutically acceptable diluent buffer, such as bacteriostatic water for injection (BWFI), phosphate-buffered saline, Ringer's solution, and/or dextrose solution. The kit may further include other materials desirable from a commercial and user standpoint, including other buffers, diluents, filters, needles, and syringes.

### EXAMPLES

### Example 1: Generation of the Self-Replicating RNA

DNA templates encoding a self-replicating RNAs can be produced in competent Escherichia coli cells transformed with a DNA plasmid. Individual bacterial colonies can be isolated and the resultant plasmid DNA amplified in E. coli cultures. Following fermentation, the plasmid DNA can be isolated using Maxiprep DNA kit and linearized by restriction digest. Restriction enzymes can then be removed using phenol/chloroform extraction and ethanol precipitation.

mRNA can be made by in vitro transcription from the linearized DNA template using a T7 RNA polymerase. Subsequently, the DNA template can be removed by DNase digestion. Enzymatic capping can be performed with Cap0 to provide functional mRNA. The resultant mRNA can then be purified and resuspended in nuclease-free water.

### Example 2: In Vitro Characterisation of the Self-Replicating RNA

The self-replicating RNAs produced in Example 1 can then be assessed for expression of the genes of interest that are expressed in the form of an antigen.

Two-fold serial dilutions of unformulated (naked) or LNP-formulated self-amplifying mRNA constructs can be either electroporated or transfected into a Baby Hamster Kidney (BHK) cell line. After about 17-19 hrs, cells can be harvested and stained for either HA, NA, NS1, NP or M1 antigen expression using anti-HA, anti-NA, anti-NS1, anti-NP or anti-M1 antibodies. The number of cells positive for antigen expression and the mean fluorescence intensities (MFIs) can be measured by FACS. Data are analysed to calculate the specific potency values (the probability of successful transfection per unit of mass of RNA) and the MFI generated.

In vitro activity and potency of unformulated RNA and LNPs can be determined by FACs based on antigen co-expression and expressed in read-outs such as FACS potentcy, encapsulation efficiency, SAM recovery, size, PDI, Zeta potential, conductivity, concentration and endotoxin levels.

### Antibody Responses

To assess antibody responses, serum can be collected at the end of study and tested by microneutralization assays and hemagglutination inhibition assay.

For all serological assays sera can be treated in the same way, with Vibrio cholerae neuraminidase, also known as receptor-destroying enzyme (RDE) (Denka Seiken Co. Ltd., Tokyo, Japan) and diluted to a starting dilution of 1:10 with PBS. Sheep serum to H5N1 virus (FDA/CBER Kensington lot nu. H5-Ag-1115) can be used as positive control sera.

### Microneutralization Assays

Microneutralization assays, short and long form are performed in a qualified mammalian cell line (proprietary 33016-PF Madin-Darby Canine Kidney (MDCK)).

### Microneutralization Assay Short Form (MN Assay SF)

Virus fluorescent focus-based microneutralization (FFA MN) assay can be performed using an in house developed protocol. RDE treated test mouse samples and positive control sera can be heat inactivated, diluted to a starting dilution of 1:40 with PBS, and fourfold serial diluted using the U-Bottom 96 well plate (BD Falcon) in neutralization medium (comprised of minimum essential medium D-MEM (GIBCO), supplemented with 1% BSA (Rockland, BSA-30), 100 U/mL penicillin and 100 μg/mL streptomycin (GIBCO)). A/turkey/Turkey/1/2005 (H5N1) virus can be diluted to ~1,000-1,500 fluorescent focus-forming units (FFU)/well (20,000-30,000 FFU/mL) in neutralization medium and added in a 1:1 ratio to diluted serum.

After incubation for 2 h at 37° C., 5% CO₂, plates (Half Area 96 well plate, Corning) containing MDCK 33016-PF cells can be inoculated with this mixture and incubated overnight for 16-18 h at 37° C. with 5% CO₂. MDCK 33016-PF cells are seeded as 3.0E4/well (3.0E6/plate) at 6-8 h earlier in the cell growth medium (comprised of D-MEM, supplemented with 10% HyClone fetal bovine serum—FBS (Gibco), 100 U/mL penicillin and 100 μg/mL streptomycin). Following the overnight incubation and prior to immunostaining, cells can then be fixed with cold mixture of acetone and methanol.

The virus can then be visualized using separate 1 h incubations at room temperature of monoclonal antibodies specific to the virus proteins of interest and Alexa Fluor 488 Goat Anti-Mouse IgG (H+L) Ab (Invitrogen cat. no. A11001) diluted in PBS buffer containing 0.05% tween-20 (Sigma) and 2% BSA (Fraction V, Calbiochem, 2960, 1194C175). Viral protein can be quantified by a CTL Immunospot analyzer (Cellular Technology Limited, Shaker Heights, Cleveland, OH), using a fluorescein isothiocyanate (FITC) fluorescence filter set with excitation and emission wavelengths of 482 and 536 nm. Fluorescent foci can be enumerated by use of software Immunospot 7.0.12.1 professional analyzer DC, using a custom analysis module.

### Microneutralization Assay Long Form (MN Assay LF)

MN assay LF is performed using an in house developed protocol. RDE treated test mouse samples and positive control sera are heat inactivated, diluted to a starting dilution of 1:40 with PBS, and twofold serial diluted using the U-Bottom 96 well plate (BD Falcon) in neutralization medium (comprised of the 30% spent growth media (Irvine Scientific) and 70% infective media (protein free media—33016 MDCK PFM; GIBCO) supplemented with 100 U/mL penicillin, 100 ug/mL streptomycin (GIBCO), and 0.33 ug/mL TPCK-trypsin (TPCK treated, Tosyl phenylalanyl chloromethyl ketone, Sigma). A virus of interest is diluted to 100TCID (tissue culture infectious dose) per well in neutralization medium and added in a 1:1 ratio to diluted serum. Serially pre-diluted serum samples are incubated with the virus and allowed to react for 1 h at 37° C., 5% CO2. In the inoculation step, plates (Cell Culture 96-well plate, Costar) containing MDCK 33016-PF cells are seeded at 3.0^{E4}/well (3.0^{E6}/plate) a day before in the antibiotic free cell growth medium (Irvine Scientific) and washed with sterile PBS, then infected with this mixture and incubated for 1 h at 37° C. with 5% CO₂. Infection is stopped by aspiration of antibody/virus mixture and cells washed with sterile PBS, inoculated with neutralizing media (100 ul/well) containing twofold serially diluted antibodies and then incubated for 5 days at 37° C. with 5% CO₂. In the final “read-out” step, detection of virus is performed by quantification of the virus using 0.5% turkey red blood cells (Lampire Biological Laboratories). The absence of infectivity constitutes a positive neutralization reaction and indicates the presence of virus-specific antibodies in the serum sample.

### Hemagglutination Inhibition (HAI) Assay

A HAI assay can be performed as previously described (WHO (2011) Manual for the laboratory diagnosis and virological surveillance of influenza: WHO Press, World Health Organization, 20 Avenue Appia, 1211 Geneva 27, Switzerland). Briefly, RDE treated test mouse samples and positive control sera are heat inactivated, diluted to a starting dilution of 1:10 with PBS, and twofold serial diluted samples (25 μl) are incubated with equal volumes of viruses (4 hemagglutinating units [HAU]) of A/turkey/Turkey/1/2005 (H5N1) at room temperature (RT) for 30 minutes. Then, an equal volume of 0.5% turkey red blood cells (Lampire Biological Laboratories) is added and incubated at RT for 30 minutes. The HAI titer can be expressed as the reciprocal of the highest dilution of the samples inhibiting hemagglutination.

### Example 3: Self-Replicating RNA Induces Cell-Mediated Immune Responses

The self-replicating RNAs are assessed for their ability to induce antigen specific T cell responses. Antigen-specific T cell detection is performed on splenic cultures. Briefly, splenocytes are dissociated in dissociation solution (MACS BSA stock 1:20 with autoMACS rinsing solution) and concentrated at 4^{E7} cells/ml. Briefly, splenocyte cultures are established in 96 well plates in T cell medium containing RPMI, NEAA, pen/strep and PME) and cultured at 37° C./5% CO₂. Anti-CD28 (clone 37.51; BD Biosciences #553294) and anti-CD107a (clone #1D4B; Biolegend #121618) are added to each well. Cell cultures are either stimulated or unstimulated. To stimulate cultures NA pep mix (JPT Peptide Technologies GmbH; PM-INFA-NATur), HA pep mix (JPT Peptide Technologies GmbH; PM-INFA-HAIndo) is added. Following 2 hours of stimulation, Golgi Plug (with brefeldin A; BD Biosciences #555029) is added to each well. Cells are incubated at 37° C. for a total of 6 hours after which the cells are transferred to 4° C. and stored overnight.

Antigen-specific T cell responses are determined using flow cytometry. Briefly, Fc block mixture (clone 2.4G2; BD Biosciences #553142) is added to each well, followed by extracellular stain (comprising Brilliant stain buffer plus (BD Biosciences #566385), ICOS BV711 (clone C398.4A; Biolegend #313548), CD44 BUV395 (clone IM7; BD Biosciences #740215), CD3 BV786 (clone 145-2C11; BD Biosciences #564379), CD4 APC-H7 (clone GK1.5, BD Biosciences #560181), CD8 AF700 (clone 53-6.7, BD Biosciences #557959) and staining buffer). Cells are stained with UltraComp eBeads (eBiosciences #01-222-42) according to the manufacturer's protocol and incubated at 4° C. for 30 mins, protected from the light. Cells are washed with staining buffer, centrifuged, resuspended in staining buffer and data acquired using a flow cytometer.

### IgG Subclass

To characterize the type of immune response generated, i.e. Th1 vs Th2 type responses, the S specific IgG1 and IgG2a IgG subclasses can be evaluated by ELISA. In addition, the ratio of IgG1/IgG2a antibodies can also be assessed.

### Example 4: Protective Effect of Immunization with Self-Replicating RNAs

To evaluate the protective effect of immunization, hamsters can be immunized with vaccines described herein at doses of 3 μg RNA/hamster or 0.3 μg RNA/hamster at Day 1 and Day 22. All animals can be challenged 28 days post the second immunization with a suitable virus intranasally and sacrificed 4 days later, where lung and nasal turbinates are collected for infectious virus measured in lungs and nasal turbinates.

In hamsters, 3.0 and 0.3 μg doses raised neutralization titer GMT as 422 and 190 respectively.

To evaluate protection of lungs from virus infection, average virus recovery from lungs are compared for hamsters immunized with vaccines of interest and control hamsters immunized by PBS.

1. A self-replicating RNA comprising a first nucleotide sequence encoding an antigen operably linked to a regulatory element, and a second nucleotide sequence encoding an adjuvant operably linked to a regulatory element, wherein the adjuvant is cathelicidin or a fragment thereof.

2. The self-replicating RNA of claim 1, wherein the regulatory element is selected from the group consisting of a promoter, an IRES and a Kozak consensus sequence.

3. The self-replicating RNA of claim 1 or 2, wherein the self-replicating RNA comprises in 5′ to 3′ order:
a) the nucleotide sequence encoding an antigen operably linked to a regulatory element; and
b) the nucleotide sequence encoding an adjuvant operably linked to a regulatory element, wherein the adjuvant is cathelicidin or a fragment thereof.

4. The self-replicating RNA of claim 1 or 2, wherein the self-replicating RNA comprises in 5′ to 3′ order:
a) the nucleotide sequence encoding an adjuvant operably linked to a regulatory element, wherein the adjuvant is cathelicidin or a fragment thereof; and
b) the nucleotide sequence encoding an antigen operably linked to a regulatory element.

5. The self-replicating RNA of claim 3, wherein the self-replicating RNA comprises in 5′ to 3′ order:
a) the nucleotide sequence encoding an antigen operably linked to a SG promoter; and
b) the nucleotide sequence encoding an adjuvant operably linked to a regulatory element selected from the group consisting of a SG promoter and an internal ribosome entry site (IRES), wherein the adjuvant is cathelicidin or a fragment thereof.

6. The self-replicating RNA of claim 4, wherein the self-replicating RNA comprises in 5′ to 3′ order:
a) the nucleotide sequence encoding an adjuvant operably linked to a SG promoter, wherein the adjuvant is cathelicidin or a fragment thereof; and
b) the nucleotide sequence encoding an antigen operably linked to a regulatory element selected from the group consisting of a SG promoter and an internal ribosome entry site (IRES).

7. The self-replicating RNA of any one of claims 1 to 6, wherein the self-replicating RNA is a polycistronic self-replicating RNA.

8. The self-replicating RNA of claim 2, wherein the promoter is a subgenomic (SG) promoter.

9. The self-replicating RNA of claim 8, wherein the SG promoter is a minimal SG promoter.

10. The self-replicating RNA of claim 8, wherein the SG promoter is an extended SG promoter.

11. The self-replicating RNA of claim 10, wherein the extended SG promoter is extended at the 5′ end with nucleotides occurring in a sequence encoding a non-structural protein of a RNA virus.

12. The self-replicating RNA of claim 10, wherein the minimal SG promoter is encoded by a sequence set forth in SEQ ID NO: 14 or 33.

13. The self-replicating RNA of claim 10, wherein the extended SG promoter is encoded by a sequence set forth in SEQ ID NOs: 15, 16 or 18.

14. The self-replicating RNA of claim 2, wherein the nucleotide sequence encoding an adjuvant is operably linked to an IRES located 5′ to the nucleotide sequence encoding the adjuvant.

15. The self-replicating RNA of claim 2 or 14, wherein the IRES is an IRES from encephalomyocarditis virus (EMCV), poliovirus (PV), human enterovirus, foot-and-mouth disease virus (FMDV), hepatitis C virus (HCV), classical swine fever virus (CSFV), murine leukemia virus (MLV), simian immunodeficiency virus (SIV), Eukaryotic translation initiation factor 4G (elF4G), Death-associated protein 5 (DAP5), cellular Myc (c-Myc), NF-κB-repressing factor (NRF), vascular endothelial growth factor (VEGF), fibroblast growth factor (FGF-2), platelet-derived growth factor B (PDGF B), Antennapedia, X-linked inhibitor of apoptosis (XIAP or Apaf-1), immunoglobulin heavy-chain binding protein BiP, or fibroblast growth factor 1a (FGF1A), GTX, or a combination thereof.

16. The self-replicating RNA of claim 15, wherein the EMCV IRES is a wild-type IRES encoded by a sequence set forth in SEQ ID NO: 17.

17. The self-replicating RNA of any one of claims 1 to 16, wherein the antigen is selected from the group consisting of influenza virus, respiratory syncytial virus, parainfluenza viruses, metapneumovirus, rhinovirus, coronaviruses, adenoviruses and bocaviruses.

18. The self-replicating RNA of any one of claims 1 to 17, wherein the cathelicidin is selected from the group consisting of dododecapeptide, indolicidin, buCATHL4A, protegrin-1, PMAP-23, BMAP-27, eCATH-2, SMAP-29, mCRAMP, rCRAMP, PMAP-36, LL-37, CAP18-FV, PMAP-37, ttLL-37, eCATH-3, Bac7 or prophenin-1 or a fragment thereof.

19. The self-replicating RNA of claim 18, wherein the nucleotide sequence encoding a cathelicidin or a fragment thereof comprises a polynucleotide sequence having at least about 70%, at least about 75%, at least about 80%, at least about 85%, at least about 90% at least about 95%, at least about 96%, at least about 97%, at least about 98% or at least about 99% identity to LL-37.

20. The self-replicating RNA of claim 19, wherein the nucleotide sequence encoding a cathelicidin or a fragment thereof is LL-37.

21. The self-replicating RNA of claim 20, wherein the nucleotide sequence encoding a cathelicidin or a fragment thereof has a sequence set forth in SEQ ID NO: 1 or 4.

22. The self-replicating RNA of any one of claims 1 to 21, wherein the self-replicating RNA is from an alphavirus.

23. The self-replicating RNA of claim 22, wherein the alphavirus is selected from the group consisting of Semliki Forest virus (SFV), Sindbis virus (SIN), and Venezuelan equine encephalitis virus (VEE) and combinations thereof.

24. An immunogenic composition comprising the self-replicating RNA of any one of claims 1 to 23.

25. The immunogenic composition of claim 24, comprising a plurality of self-replicating RNAs of any one of claims 1 to 23, wherein each self-replicating RNA encodes different polypeptide sequences.

26. The immunogenic composition of claim 24, comprising a plurality of self-replicating RNAs of any one of claims 1 to 23, wherein each self-replicating RNA encodes the same polypeptide sequence.

27. The immunogenic composition of any one of claims 24 to 26, wherein the immunogenic composition further comprises a cathelicidin polypeptide or a fragment thereof.

28. An immunogenic composition comprising:
a) a self-replicating RNA comprising a first nucleotide sequence encoding an antigen operably linked to a regulatory element; and
b) a cathelicidin polypeptide or a fragment thereof.

29. The immunogenic composition of claim 28, further comprising an additional RNA encoding:
(i) one or more antigens;
(ii) one or more immune response enhancers;
(iii) one or more chemoattractants; and/or
(iv) one or more targeting molecules.

30. The immunogenic composition of claim 28 or 29, wherein the regulatory element is a promoter, an internal ribosome entry site (IRES), a Kozak consensus sequence or a combination thereof.

31. The immunogenic composition of claim 30, wherein the promoter is a subgenomic (SG) promoter.

32. The immunogenic composition of claim 31, wherein the SG promoter is a minimal SG promoter or an extended SG promoter.

33. The immunogenic composition of claim 32, wherein the extended SG promoter is extended at the 5′ end with nucleotides occurring in a sequence encoding a non-structural protein of a RNA virus.

34. The immunogenic composition of claim 32, wherein the minimal SG promoter is encoded by a sequence set forth in SEQ ID NO: 14 or 33.

35. The immunogenic composition of claim 32, wherein the extended SG promoter is encoded by a sequence set forth in SEQ ID NOs: 15, 16 or 18.

36. The immunogenic composition of claim 30, wherein the nucleotide sequence encoding an adjuvant is operably linked to an IRES located 5′ to the nucleotide sequence encoding the adjuvant.

37. The immunogenic composition of claim 32, wherein the IRES is an IRES from encephalomyocarditis virus (EMCV), poliovirus (PV), human enterovirus, foot-and-mouth disease virus (FMDV), hepatitis C virus (HCV), classical swine fever virus (CSFV), murine leukemia virus (MLV), simian immunodeficiency virus (SIV), Eukaryotic translation initiation factor 4G (elF4G), Death-associated protein 5 (DAP5), cellular Myc (c-Myc), NF-κB-repressing factor (NRF), vascular endothelial growth factor (VEGF), fibroblast growth factor (FGF-2), platelet-derived growth factor B (PDGF B), Antennapedia, X-linked inhibitor of apoptosis (XIAP or Apaf-1), immunoglobulin heavy-chain binding protein BiP, or fibroblast growth factor 1a (FGF1A), GTX, or a combination thereof.

38. The immunogenic composition of claim 37, wherein the EMCV IRES is a wild-type IRES encoded by a sequence set forth in SEQ ID NO: 17.

39. The immunogenic composition of any one of claims 28 to 38, wherein the antigen is selected from the group consisting of influenza virus, respiratory syncytial virus, parainfluenza viruses, metapneumovirus, rhinovirus, coronaviruses, adenoviruses and bocaviruses.

40. The immunogenic composition of any one of claims 28 to 39, wherein the cathelicidin polypeptide or a fragment thereof is selected from the group consisting of dododecapeptide, indolicidin, buCATHL4A, protegrin-1, PMAP-23, BMAP-27, eCATH-2, SMAP-29, mCRAMP, rCRAMP, PMAP-36, LL-37, CAP18-FV, PMAP-37, ttLL-37, eCATH-3, Bac7 or prophenin-1 or a fragment thereof.

41. The immunogenic composition of claim 40, wherein the cathelicidin polypeptide or a fragment thereof comprises a polynucleotide sequence having at least about 70%, at least about 75%, at least about 80%, at least about 85%, at least about 90% at least about 95%, at least about 96%, at least about 97%, at least about 98% or at least about 99% sequence identity to LL-37.

42. The immunogenic composition of claim 41, wherein the cathelicidin polypeptide or a fragment thereof is LL-37.

43. The immunogenic composition of claim 42, wherein the cathelicidin polypeptide or a fragment thereof has a sequence set forth in SEQ ID NO: 3 or 6.

44. The immunogenic composition of any one of claims 28 to 43, wherein the self-replicating RNA is from an alphavirus.

45. The immunogenic composition of claim 44, wherein the alphavirus is selected from the group consisting of Semliki Forest virus (SFV), Sindbis virus (SIN), and Venezuelan equine encephalitis virus (VEE) and combinations thereof.

46. A pharmaceutical composition comprising an immunogenic composition of any one of claims 24 to 45 and a pharmaceutically acceptable carrier.

47. The pharmaceutical composition of claim 46, wherein the self-replicating RNA is encapsulated in, bound to or adsorbed on a lipid nanoparticle (LNP), a polymeric microparticle or an oil-in-water emulsion.

48. The pharmaceutical composition of any one of claims 46 to 47, wherein each RNA is formulated together in the LNP.

49. The pharmaceutical composition of any one of claims 46 to 47, wherein each RNA is formulated separately in the LNP.

50. The immunogenic composition of any one of claims 24 to 45 or the pharmaceutical composition of any one of claims 46 to 49, for use as a vaccine.

51. A vaccine comprising the pharmaceutical composition of any one of claims 46 to 49 or the immunogenic composition of any one of claims 24 to 45.

52. A polynucleotide encoding the self-replicating RNA of any one of claims 1 to 23.

53. The polynucleotide of claim 52, wherein the polynucleotide is a recombinant DNA.

54. The polynucleotide of claim 53, wherein the recombinant DNA is a plasmid.

55. The polynucleotide of claim 54, wherein the plasmid comprises a sequence set forth in SEQ ID NO: 32.

56. A polynucleotide comprising:
a) a first nucleotide sequence encoding an antigen operably linked to a regulatory element; and
b) a second nucleotide sequence encoding an adjuvant operably linked to a regulatory element selected from the group consisting of a SG promoter and an internal ribosome entry site (IRES),
wherein the adjuvant is cathelicidin or a fragment thereof.

57. The polynucletide of claim 56, wherein the polynucleotide comprises, in order from 5′ to 3′:
a) the first nucleotide sequence encoding an antigen operably linked to a regulatory element; and
b) the second nucleotide sequence encoding an adjuvant operably linked to an IRES or a SG promoter,
wherein the adjuvant is a cathelicidin or a fragment thereof.

58. A conventional mRNA (cRNA) comprising:
a) a first nucleotide sequence encoding an antigen operably linked to a regulatory element; and
b) a second nucleotide sequence encoding an adjuvant operably linked to a regulatory element selected from the group consisting of a SG promoter and an internal ribosome entry site (IRES),
wherein the adjuvant is a cathelicidin or a fragment thereof.

59. The cRNA of claim 58, wherein the cRNA comprises, in order from 5′ to 3′:
a) the first nucleotide sequence encoding an antigen operably linked to a regulatory element; and
b) the second nucleotide sequence encoding an adjuvant operably linked to an IRES or a SG promoter,
wherein the adjuvant is a cathelicidin or a fragment thereof.

60. The polynucleotide of any one of claims 53 to 57, or the cRNA of claim 58 or 59, wherein the first nucleotide sequence is operably linked to a regulatory element selected from the group consisting of a Kozak consensus sequence, an IRES, a SG promoter and combinations thereof.

61. A method of treating or preventing or delaying progression of a disease or condition in a subject in need thereof, the method comprising administering the pharmaceutical composition of any one of claims 46 to 49, the immunogenic composition any one of claims 24 to 45 or the vaccine of claim 51 to the subject.

62. Use of the pharmaceutical composition of any one of claims 46 to 49, the immunogenic composition any one of claims 24 to 45 or the vaccine of claim 51 in the manufacture of a medicament for treating or preventing or delaying progression of a disease or condition in a subject in need thereof.

63. The pharmaceutical composition of any one of claims 46 to 49, the immunogenic composition any one of claims 24 to 45 or the vaccine of claim 51 for use in treating or preventing or delaying progression of a disease or condition in a subject in need thereof.

64. A method of inducing an immune response in a subject, the method comprising administering the pharmaceutical composition of any one of claims 46 to 49, the immunogenic composition any one of claims 24 to 45 or the vaccine of claim 51 to a subject in need thereof.

65. Use of the pharmaceutical composition of any one of claims 46 to 49, the immunogenic composition any one of claims 24 to 45 or the vaccine of claim 51 in the manufacture of a medicament for inducing an immune response in a subject in need thereof.

66. The pharmaceutical composition of any one of claims 46 to 49, the immunogenic composition any one of claims 24 to 45 or the vaccine of claim 51 for use in inducing an immune response in a subject in need thereof.

67. The method of claim 64, the use of claim 65, or the self-replicating RNA, pharmaceutical composition, immunogenic composition or vaccine for use of claim 66,
wherein the immune response is a humoral and/or a cell-mediated immune response.

68. A method for reducing viral load in a subject comprising administering pharmaceutical composition of any one of claims 46 to 49, the immunogenic composition any one of claims 24 to 45 or the vaccine of claim 51 to the subject in need thereof.

69. Use of the pharmaceutical composition of any one of claims 46 to 49, the immunogenic composition any one of claims 24 to 45 or the vaccine of claim 51 in the preparation of a medicament for reducing viral load in a subject in need thereof.

70. The pharmaceutical composition of any one of claims 46 to 49, the immunogenic composition any one of claims 24 to 45 or the vaccine of claim 51 for use in reducing viral load in a subject in need thereof.

71. The method of any one of claims 61, 64, 67 or 68, the use of any one of claims 62, 65, 67 or 69, or the pharmaceutical composition, immunogenic composition or vaccine for use of any one of claims 63, 65, 67 or 70, wherein the subject is a human of 18 years of age or older.

72. The method of any one of claims 61, 64, 67 or 68, the use of any one of claims 62, 65, 67 or 69, or the pharmaceutical composition, immunogenic composition or vaccine for use of any one of claims 63, 65, 67 or 70, wherein the vaccine or composition is administered in a one dose regimen.

73. The method of any one of claims 61, 64, 67 or 68, the use of any one of claims 62, 65, 67 or 69, or the pharmaceutical composition, immunogenic composition or vaccine for use of any one of claims 63, 65, 67 or 70, wherein the vaccine or composition is administered in a two, three or four dose regimen, wherein the doses are administered about 1, 2 or 3 months apart.

74. A kit comprising:
(a) a self-replicating RNA of any one of claims 1 to 23, pharmaceutical composition of any one of claims 46 to 49, the immunogenic composition any one of claims 24 to 45 or the vaccine of claim 51;
(b) instructions for use thereof; and optionally
(c) a pharmaceutically acceptable carrier, excipient or diluent.
